SEQUENCE LISTING (574 sequences)

SEQ ID NO: 1 - DNA, 1725 bp, respiratory syncytival virus
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 2 - DNA, 1575 bp, respiratory syncytial virus
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataactatagaattaagtaatatcaagaaaaataagtgtaatggaacagatgctaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaattaa

SEQ ID NO: 3 - DNA, 1725 bp, Artificial Sequence
  Optimised Sequence
atggagttgctaatcctcaaagcaaatgcaattaccacaatcctcactgcggtcaccttttgttttgcttctggtcaaaacatcactgaagaattttatcaatcaacatgcagtgcagttagcaaaggatatcttagtgctctgagaaccggttggtataccagtgttataactatagaattaagtaatatcaagaaaaataagtgtaatggtaccgatgctaaggtaaaattgataaaacaagaattagataaatataaaaatgctgtaacagaattgcagttgctcatgcagtcgacacaagcaacaaacaatcgagccagaagagaactacctaggtttatgaattatacactcaacaatgccaaaaaaaccaatgtaacactttcgaagaaaaggaaaagaagatttcttggttttttgttaggtgttggatccgcaatcgccagtggcgttgctgtatctaaggtcctgcatctagagggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgacgcgtcaatatctcaagtcaacgagaagattaaccagagcttagcatttattcgtaaatccgatgaattattacataatgtaaatgctgggaagagcaccacaaatatcatgataactactataattatagtgattatagtaatattgttatcattaattgctgttggactgctcttatactgtaaggccagatctacaccagtcacactaagcaaagatcaactgagtggtataaataatattgcatttagtaactaa

SEQ ID NO: 4 - DNA, 1575 bp, Artificial Sequence
  Optimised Sequence
atggagttgctaatcctcaaagcaaatgcaattaccacaatcctcactgcggtcaccttttgttttgcttctggtcaaaacatcactgaagaattttatcaatcaacatgcagtgcagttagcaaaggatatcttagtgctctgagaaccggttggtataccagtgttataactatagaattaagtaatatcaagaaaaataagtgtaatggtaccgatgctaaggtaaaattgataaaacaagaattagataaatataaaaatgctgtaacagaattgcagttgctcatgcagtcgacacaagcaacaaacaatcgagccagaagagaactacctaggtttatgaattatacactcaacaatgccaaaaaaaccaatgtaacactttcgaagaaaaggaaaagaagatttcttggttttttgttaggtgttggatccgcaatcgccagtggcgttgctgtatctaaggtcctgcatctagagggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgacgcgtcaatatctcaagtcaacgagaagattaaccagagcttagcatttattcgtaaatccgatgaattattacataatgtaaatgctgggaagagcaccacaaattaa

SEQ ID NO: 5 - DNA, 1725 bp, Artificial Sequence
  Optimised Sequence
atggagctgctgatcctgaaggccaacgccatcaccaccatcctgaccgcggtgaccttctgcttcgcctctggccagaacatcactgaggagttctaccagagcacttgttccgctgtgagcaagggctacctgagcgccctgaggaccggttggtacaccagcgtgatcaccatcgagctgagcaacatcaagaagaacaagtgcaacggcaccgacgccaaggtgaagctgatcaagcaagagctggacaagtacaagaacgccgtgaccgagctgcaactgctgatgcagtcgactcaagccaccaacaacagagcccgcagagagctgccccgcttcatgaactacaccctgaacaacgccaagaagaccaacgtgaccctgtccaagaagaggaagcgccgcttcctgggcttcctgctgggcgtgggctccgccattgccagtggcgtggccgtgtccaaggtgctgcacctggagggcgaggtgaacaagatcaagagtgccctgctgtccactaacaaggccgtggtgagcctgagcaacggcgtgagtgtgctgactagcaaggtgctggacctgaagaactacatcgacaagcaattgctgcccatcgtgaacaagcagtcctgtagcatctccaacatcgagactgtgatcgagttccagcagaagaacaaccgcctgctggaaatcacccgggagttcagtgtgaacgctggcgtgaccactcctgtctccacctacatgctgaccaacagcgagctgctgagcctgatcaacgacatgcccatcaccaacgaccagaagaagcttatgtccaacaacgtgcagatcgtgaggcagcagagctactccatcatgagcatcatcaaggaggaggtgctggcctatgtggtgcagctgcccctgtacggcgtcatcgataccccttgctggaagctgcacaccagccccctgtgcaccaccaacaccaaggagggcagcaacatctgcctgacccggaccgaccgcggctggtactgtgacaacgctggctcggtgagcttcttccctcaagctgaaacctgcaaggtccagagcaacagagtgttctgtgacaccatgaactccctgaccctgccctccgaggtgaacctgtgcaacgtggatatcttcaaccccaagtatgactgcaagatcatgacctccaagaccgatgtctcgagctccgtgatcaccagcctgggcgccatcgtgtcctgctatggcaagaccaagtgcaccgccagcaacaagaaccggggcatcatcaagaccttcagcaatgggtgcgactacgtttcgaacaagggcgtggacactgtgtccgtgggcaacaccctgtactacgtgaacaagcaagagggcaagagcctgtatgtgaagggcgagcccatcatcaacttctacgaccccctggtgttcccctccgacgaattcgacgcctccattagccaagtcaacgagaagatcaaccagagcctggccttcatccgcaagtccgacgagctgctgcacaacgtcaacgctggcaagagcaccaccaacatcatgatcaccaccatcatcatcgtgatcatcgtgatcctgctgagcctgatcgccgtgggcctgctgctgtactgcaaggcccggagcactcccgtgaccctgagcaaggaccagctgagcggcatcaacaacatcgccttcagcaactga

SEQ ID NO: 6 - DNA, 1575 bp, Artificial Sequence
  Optimised Sequence
atggagctgctgatcctgaaggccaacgccatcaccaccatcctgaccgcggtgaccttctgcttcgcctctggccagaacatcactgaggagttctaccagagcacttgttccgctgtgagcaagggctacctgagcgccctgaggaccggttggtacaccagcgtgatcaccatcgagctgagcaacatcaagaagaacaagtgcaacggcaccgacgccaaggtgaagctgatcaagcaagagctggacaagtacaagaacgccgtgaccgagctgcaactgctgatgcagtcgactcaagccaccaacaacagagcccgcagagagctgccccgcttcatgaactacaccctgaacaacgccaagaagaccaacgtgaccctgtccaagaagaggaagcgccgcttcctgggcttcctgctgggcgtgggctccgccattgccagtggcgtggccgtgtccaaggtgctgcacctggagggcgaggtgaacaagatcaagagtgccctgctgtccactaacaaggccgtggtgagcctgagcaacggcgtgagtgtgctgactagcaaggtgctggacctgaagaactacatcgacaagcaattgctgcccatcgtgaacaagcagtcctgtagcatctccaacatcgagactgtgatcgagttccagcagaagaacaaccgcctgctggaaatcacccgggagttcagtgtgaacgctggcgtgaccactcctgtctccacctacatgctgaccaacagcgagctgctgagcctgatcaacgacatgcccatcaccaacgaccagaagaagcttatgtccaacaacgtgcagatcgtgaggcagcagagctactccatcatgagcatcatcaaggaggaggtgctggcctatgtggtgcagctgcccctgtacggcgtcatcgataccccttgctggaagctgcacaccagccccctgtgcaccaccaacaccaaggagggcagcaacatctgcctgacccggaccgaccgcggctggtactgtgacaacgctggctcggtgagcttcttccctcaagctgaaacctgcaaggtccagagcaacagagtgttctgtgacaccatgaactccctgaccctgccctccgaggtgaacctgtgcaacgtggatatcttcaaccccaagtatgactgcaagatcatgacctccaagaccgatgtctcgagctccgtgatcaccagcctgggcgccatcgtgtcctgctatggcaagaccaagtgcaccgccagcaacaagaaccggggcatcatcaagaccttcagcaatgggtgcgactacgtttcgaacaagggcgtggacactgtgtccgtgggcaacaccctgtactacgtgaacaagcaagagggcaagagcctgtatgtgaagggcgagcccatcatcaacttctacgaccccctggtgttcccctccgacgaattcgacgcctccattagccaagtcaacgagaagatcaaccagagcctggccttcatccgcaagtccgacgagctgctgcacaacgtcaacgctggcaagagcaccaccaactga

SEQ ID NO: 7 - protein, 574 aa, respiratory syncytial virus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSN

SEQ ID NO: 8 - protein, 524 aa, respiratory syncytial virus
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNRARRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTN

SEQ ID NO: 9 - protein, 10 aa, respiratory syncytial virus
CFASGQNITE

SEQ ID NO: 10 - protein, 10 aa, respiratory syncytial virus
FASGQNITEE

SEQ ID NO: 11 - protein, 10 aa, respiratory syncytial virus
ASGQNITEEF

SEQ ID NO: 12 - protein, 10 aa, respiratory syncytial virus
SGQNITEEFY

SEQ ID NO: 13 - protein, 10 aa, respiratory syncytial virus
GQNITEEFYQ

SEQ ID NO: 14 - protein, 10 aa, respiratory syncytial virus
QNITEEFYQS

SEQ ID NO: 15 - protein, 10 aa, respiratory syncytial virus
NITEEFYQST

SEQ ID NO: 16 - protein, 10 aa, respiratory syncytial virus
ITEEFYQSTC

SEQ ID NO: 17 - protein, 10 aa, respiratory syncytial virus
TEEFYQSTCS

SEQ ID NO: 18 - protein, 10 aa, respiratory syncytial virus
EEFYQSTCSA

SEQ ID NO: 19 - protein, 10 aa, respiratory syncytial virus
EFYQSTCSAV

SEQ ID NO: 20 - protein, 10 aa, respiratory syncytial virus
FYQSTCSAVS

SEQ ID NO: 21 - protein, 10 aa, respiratory syncytial virus
YQSTCSAVSK

SEQ ID NO: 22 - protein, 10 aa, respiratory syncytial virus
QSTCSAVSKG

SEQ ID NO: 23 - protein, 10 aa, respiratory syncytial virus
STCSAVSKGY

SEQ ID NO: 24 - protein, 10 aa, respiratory syncytial virus
TCSAVSKGYL

SEQ ID NO: 25 - protein, 10 aa, respiratory syncytial virus
CSAVSKGYLS

SEQ ID NO: 26 - protein, 10 aa, respiratory syncytial virus
SAVSKGYLSA

SEQ ID NO: 27 - protein, 10 aa, respiratory syncytial virus
AVSKGYLSAL

SEQ ID NO: 28 - protein, 10 aa, respiratory syncytial virus
VSKGYLSALR

SEQ ID NO: 29 - protein, 10 aa, respiratory syncytial virus
SKGYLSALRT

SEQ ID NO: 30 - protein, 10 aa, respiratory syncytial virus
KGYLSALRTG

SEQ ID NO: 31 - protein, 10 aa, respiratory syncytial virus
GYLSALRTGW

SEQ ID NO: 32 - protein, 10 aa, respiratory syncytial virus
YLSALRTGWY

SEQ ID NO: 33 - protein, 10 aa, respiratory syncytial virus
LSALRTGWYT

SEQ ID NO: 34 - protein, 10 aa, respiratory syncytial virus
SALRTGWYTS

SEQ ID NO: 35 - protein, 10 aa, respiratory syncytial virus
ALRTGWYTSV

SEQ ID NO: 36 - protein, 10 aa, respiratory syncytial virus
LRTGWYTSVI

SEQ ID NO: 37 - protein, 10 aa, respiratory syncytial virus
RTGWYTSVIT

SEQ ID NO: 38 - protein, 10 aa, respiratory syncytial virus
TGWYTSVITI

SEQ ID NO: 39 - protein, 10 aa, respiratory syncytial virus
GWYTSVITIE

SEQ ID NO: 40 - protein, 10 aa, respiratory syncytial virus
WYTSVITIEL

SEQ ID NO: 41 - protein, 10 aa, respiratory syncytial virus
TSVITIELSN

SEQ ID NO: 42 - protein, 10 aa, respiratory syncytial virus
SVITIELSNI

SEQ ID NO: 43 - protein, 10 aa, respiratory syncytial virus
SVITIELSNI

SEQ ID NO: 44 - protein, 10 aa, respiratory syncytial virus
VITIELSNIK

SEQ ID NO: 45 - protein, 10 aa, respiratory syncytial virus
ITIELSNIKK

SEQ ID NO: 46 - protein, 10 aa, respiratory syncytial virus
TIELSNIKKN

SEQ ID NO: 47 - protein, 10 aa, respiratory syncytial virus
IELSNIKKNK

SEQ ID NO: 48 - protein, 10 aa, respiratory syncytial virus
ELSNIKKNKC

SEQ ID NO: 49 - protein, 10 aa, respiratory syncytial virus
LSNIKKNKCN

SEQ ID NO: 50 - protein, 10 aa, respiratory syncytial virus
SNIKKNKCNG

SEQ ID NO: 51 - protein, 10 aa, respiratory syncytial virus
NIKKNKCNGT

SEQ ID NO: 52 - protein, 10 aa, respiratory syncytial virus
IKKNKCNGTD

SEQ ID NO: 53 - protein, 10 aa, respiratory syncytial virus
KKNKCNGTDA

SEQ ID NO: 54 - protein, 10 aa, respiratory syncytial virus
KNKCNGTDAK

SEQ ID NO: 55 - protein, 10 aa, respiratory syncytial virus
NKCNGTDAKV

SEQ ID NO: 56 - protein, 10 aa, respiratory syncytial virus
KCNGTDAKVK

SEQ ID NO: 57 - protein, 10 aa, respiratory syncytial virus
CNGTDAKVKL

SEQ ID NO: 58 - protein, 10 aa, respiratory syncytial virus
NGTDAKVKLI

SEQ ID NO: 59 - protein, 10 aa, respiratory syncytial virus
GTDAKVKLIK

SEQ ID NO: 60 - protein, 10 aa, respiratory syncytial virus
TDAKVKLIKQ

SEQ ID NO: 61 - protein, 10 aa, respiratory syncytial virus
DAKVKLIKQE

SEQ ID NO: 62 - protein, 10 aa, respiratory syncytial virus
AKVKLIKQEL

SEQ ID NO: 63 - protein, 10 aa, respiratory syncytial virus
KVKLIKQELD

SEQ ID NO: 64 - protein, 10 aa, respiratory syncytial virus
VKLIKQELDK

SEQ ID NO: 65 - protein, 10 aa, respiratory syncytial virus
KLIKQELDKY

SEQ ID NO: 66 - protein, 10 aa, respiratory syncytial virus
LIKQELDKYK

SEQ ID NO: 67 - protein, 10 aa, respiratory syncytial virus
IKQELDKYKN

SEQ ID NO: 68 - protein, 10 aa, respiratory syncytial virus
KQELDKYKNA

SEQ ID NO: 69 - protein, 10 aa, respiratory syncytial virus
QELDKYKNAV

SEQ ID NO: 70 - protein, 10 aa, respiratory syncytial virus
ELDKYKNAVT

SEQ ID NO: 71 - protein, 10 aa, respiratory syncytial virus
LDKYKNAVTE

SEQ ID NO: 72 - protein, 10 aa, respiratory syncytial virus
DKYKNAVTEL

SEQ ID NO: 73 - protein, 10 aa, respiratory syncytial virus
KYKNAVTELQ

SEQ ID NO: 74 - protein, 10 aa, respiratory syncytial virus
YKNAVTELQL

SEQ ID NO: 75 - protein, 10 aa, respiratory syncytial virus
KNAVTELQLL

SEQ ID NO: 76 - protein, 10 aa, respiratory syncytial virus
NAVTELQLLM

SEQ ID NO: 77 - protein, 10 aa, respiratory syncytial virus
AVTELQLLMQ

SEQ ID NO: 78 - protein, 10 aa, respiratory syncytial virus
VTELQLLMQS

SEQ ID NO: 79 - protein, 10 aa, respiratory syncytial virus
TELQLLMQST

SEQ ID NO: 80 - protein, 10 aa, respiratory syncytial virus
ELQLLMQSTQ

SEQ ID NO: 81 - protein, 10 aa, respiratory syncytial virus
ELQLLMQSTQ

SEQ ID NO: 82 - protein, 10 aa, respiratory syncytial virus
QLLMQSTQAT

SEQ ID NO: 83 - protein, 10 aa, respiratory syncytial virus
LLMQSTQATN

SEQ ID NO: 84 - protein, 10 aa, respiratory syncytial virus
LMQSTQATNN

SEQ ID NO: 85 - protein, 10 aa, respiratory syncytial virus
MQSTQATNNR

SEQ ID NO: 86 - protein, 10 aa, respiratory syncytial virus
QSTQATNNRA

SEQ ID NO: 87 - protein, 10 aa, respiratory syncytial virus
STQATNNRAR

SEQ ID NO: 88 - protein, 10 aa, respiratory syncytial virus
TQATNNRARR

SEQ ID NO: 89 - protein, 10 aa, respiratory syncytial virus
QATNNRARRE

SEQ ID NO: 90 - protein, 10 aa, respiratory syncytial virus
ATNNRARREL

SEQ ID NO: 91 - protein, 10 aa, respiratory syncytial virus
TNNRARRELP

SEQ ID NO: 92 - protein, 10 aa, respiratory syncytial virus
NNRARRELPR

SEQ ID NO: 93 - protein, 10 aa, respiratory syncytial virus
NRARRELPRF

SEQ ID NO: 94 - protein, 10 aa, respiratory syncytial virus
RARRELPRFM

SEQ ID NO: 95 - protein, 10 aa, respiratory syncytial virus
ARRELPRFMN

SEQ ID NO: 96 - protein, 10 aa, respiratory syncytial virus
RRELPRFMNY

SEQ ID NO: 97 - protein, 10 aa, respiratory syncytial virus
RELPRFMNYT

SEQ ID NO: 98 - protein, 10 aa, respiratory syncytial virus
ELPRFMNYTL

SEQ ID NO: 99 - protein, 10 aa, respiratory syncytial virus
LPRFMNYTLN

SEQ ID NO: 100 - protein, 10 aa, respiratory syncytial virus
PRFMNYTLNN

SEQ ID NO: 101 - protein, 10 aa, respiratory syncytial virus
RFMNYTLNNA

SEQ ID NO: 102 - protein, 10 aa, respiratory syncytial virus
FMNYTLNNAK

SEQ ID NO: 103 - protein, 10 aa, respiratory syncytial virus
MNYTLNNAKK

SEQ ID NO: 104 - protein, 10 aa, respiratory syncytial virus
NYTLNNAKKT

SEQ ID NO: 105 - protein, 10 aa, respiratory syncytial virus
YTLNNAKKTN

SEQ ID NO: 106 - protein, 10 aa, respiratory syncytial virus
TLNNAKKTNV

SEQ ID NO: 107 - protein, 10 aa, respiratory syncytial virus
LNNAKKTNVT

SEQ ID NO: 108 - protein, 10 aa, respiratory syncytial virus
NNAKKTNVTL

SEQ ID NO: 109 - protein, 10 aa, respiratory syncytial virus
NAKKTNVTLS

SEQ ID NO: 110 - protein, 10 aa, respiratory syncytial virus
AKKTNVTLSK

SEQ ID NO: 111 - protein, 10 aa, respiratory syncytial virus
KKTNVTLSKK

SEQ ID NO: 112 - protein, 10 aa, respiratory syncytial virus
KTNVTLSKKR

SEQ ID NO: 113 - protein, 10 aa, respiratory syncytial virus
TNVTLSKKRK

SEQ ID NO: 114 - protein, 10 aa, respiratory syncytial virus
NVTLSKKRKR

SEQ ID NO: 115 - protein, 10 aa, respiratory syncytial virus
VTLSKKRKRR

SEQ ID NO: 116 - protein, 10 aa, respiratory syncytial virus
TLSKKRKRRF

SEQ ID NO: 117 - protein, 10 aa, respiratory syncytial virus
LSKKRKRRFL

SEQ ID NO: 118 - protein, 10 aa, respiratory syncytial virus
SKKRKRRFLG

SEQ ID NO: 119 - protein, 10 aa, respiratory syncytial virus
KKRKRRFLGF

SEQ ID NO: 120 - protein, 10 aa, respiratory syncytial virus
KRKRRFLGFL

SEQ ID NO: 121 - protein, 10 aa, respiratory syncytial virus
RKRRFLGFLL

SEQ ID NO: 122 - protein, 10 aa, respiratory syncytial virus
KRRFLGFLLG

SEQ ID NO: 123 - protein, 10 aa, respiratory syncytial virus
RRFLGFLLGV

SEQ ID NO: 124 - protein, 10 aa, respiratory syncytial virus
RFLGFLLGVG

SEQ ID NO: 125 - protein, 10 aa, respiratory syncytial virus
FLGFLLGVGS

SEQ ID NO: 126 - protein, 10 aa, respiratory syncytial virus
LGFLLGVGSA

SEQ ID NO: 127 - protein, 10 aa, respiratory syncytial virus
GFLLGVGSAI

SEQ ID NO: 128 - protein, 10 aa, respiratory syncytial virus
FLLGVGSAIA

SEQ ID NO: 129 - protein, 10 aa, respiratory syncytial virus
LLGVGSAIAS

SEQ ID NO: 130 - protein, 10 aa, respiratory syncytial virus
LGVGSAIASG

SEQ ID NO: 131 - protein, 10 aa, respiratory syncytial virus
GVGSAIASGV

SEQ ID NO: 132 - protein, 10 aa, respiratory syncytial virus
VGSAIASGVA

SEQ ID NO: 133 - protein, 10 aa, respiratory syncytial virus
GSAIASGVAV

SEQ ID NO: 134 - protein, 10 aa, respiratory syncytial virus
SAIASGVAVS

SEQ ID NO: 135 - protein, 10 aa, respiratory syncytial virus
AIASGVAVSK

SEQ ID NO: 136 - protein, 10 aa, respiratory syncytial virus
IASGVAVSKV

SEQ ID NO: 137 - protein, 10 aa, respiratory syncytial virus
ASGVAVSKVL

SEQ ID NO: 138 - protein, 10 aa, respiratory syncytial virus
SGVAVSKVLH

SEQ ID NO: 139 - protein, 10 aa, respiratory syncytial virus
GVAVSKVLHL

SEQ ID NO: 140 - protein, 10 aa, respiratory syncytial virus
VAVSKVLHLE

SEQ ID NO: 141 - protein, 10 aa, respiratory syncytial virus
VAVSKVLHLE

SEQ ID NO: 142 - protein, 10 aa, respiratory syncytial virus
VSKVLHLEGE

SEQ ID NO: 143 - protein, 10 aa, respiratory syncytial virus
SKVLHLEGEV

SEQ ID NO: 144 - protein, 10 aa, respiratory syncytial virus
KVLHLEGEVN

SEQ ID NO: 145 - protein, 10 aa, respiratory syncytial virus
VLHLEGEVNK

SEQ ID NO: 146 - protein, 10 aa, respiratory syncytial virus
LHLEGEVNKI

SEQ ID NO: 147 - protein, 10 aa, respiratory syncytial virus
HLEGEVNKIK

SEQ ID NO: 148 - protein, 10 aa, respiratory syncytial virus
LEGEVNKIKS

SEQ ID NO: 149 - protein, 10 aa, respiratory syncytial virus
EGEVNKIKSA

SEQ ID NO: 150 - protein, 10 aa, respiratory syncytial virus
GEVNKIKSAL

SEQ ID NO: 151 - protein, 10 aa, respiratory syncytial virus
EVNKIKSALL

SEQ ID NO: 152 - protein, 10 aa, respiratory syncytial virus
VNKIKSALLS

SEQ ID NO: 153 - protein, 10 aa, respiratory syncytial virus
NKIKSALLST

SEQ ID NO: 154 - protein, 10 aa, respiratory syncytial virus
KIKSALLSTN

SEQ ID NO: 155 - protein, 10 aa, respiratory syncytial virus
IKSALLSTNK

SEQ ID NO: 156 - protein, 10 aa, respiratory syncytial virus
KSALLSTNKA

SEQ ID NO: 157 - protein, 10 aa, respiratory syncytial virus
SALLSTNKAV

SEQ ID NO: 158 - protein, 10 aa, respiratory syncytial virus
ALLSTNKAVV

SEQ ID NO: 159 - protein, 10 aa, respiratory syncytial virus
LLSTNKAVVS

SEQ ID NO: 160 - protein, 10 aa, respiratory syncytial virus
LSTNKAVVSL

SEQ ID NO: 161 - protein, 10 aa, respiratory syncytial virus
STNKAVVSLS

SEQ ID NO: 162 - protein, 10 aa, respiratory syncytial virus
TNKAVVSLSN

SEQ ID NO: 163 - protein, 10 aa, respiratory syncytial virus
NKAVVSLSNG

SEQ ID NO: 164 - protein, 10 aa, respiratory syncytial virus
KAVVSLSNGV

SEQ ID NO: 165 - protein, 10 aa, respiratory syncytial virus
AVVSLSNGVS

SEQ ID NO: 166 - protein, 10 aa, respiratory syncytial virus
VVSLSNGVSV

SEQ ID NO: 167 - protein, 10 aa, respiratory syncytial virus
VSLSNGVSVL

SEQ ID NO: 168 - protein, 10 aa, respiratory syncytial virus
SLSNGVSVLT

SEQ ID NO: 169 - protein, 10 aa, respiratory syncytial virus
LSNGVSVLTS

SEQ ID NO: 170 - protein, 10 aa, respiratory syncytial virus
SNGVSVLTSK

SEQ ID NO: 171 - protein, 10 aa, respiratory syncytial virus
NGVSVLTSKV

SEQ ID NO: 172 - protein, 10 aa, respiratory syncytial virus
GVSVLTSKVL

SEQ ID NO: 173 - protein, 10 aa, respiratory syncytial virus
VSVLTSKVLD

SEQ ID NO: 174 - protein, 10 aa, respiratory syncytial virus
SVLTSKVLDL

SEQ ID NO: 175 - protein, 10 aa, respiratory syncytial virus
VLTSKVLDLK

SEQ ID NO: 176 - protein, 10 aa, respiratory syncytial virus
LTSKVLDLKN

SEQ ID NO: 177 - protein, 10 aa, respiratory syncytial virus
TSKVLDLKNY

SEQ ID NO: 178 - protein, 10 aa, respiratory syncytial virus
SKVLDLKNYI

SEQ ID NO: 179 - protein, 10 aa, respiratory syncytial virus
KVLDLKNYID

SEQ ID NO: 180 - protein, 10 aa, respiratory syncytial virus
VLDLKNYIDK

SEQ ID NO: 181 - protein, 10 aa, respiratory syncytial virus
LDLKNYIDKQ

SEQ ID NO: 182 - protein, 10 aa, respiratory syncytial virus
DLKNYIDKQL

SEQ ID NO: 183 - protein, 10 aa, respiratory syncytial virus
LKNYIDKQLL

SEQ ID NO: 184 - protein, 10 aa, respiratory syncytial virus
KNYIDKQLLP

SEQ ID NO: 185 - protein, 10 aa, respiratory syncytial virus
NYIDKQLLPI

SEQ ID NO: 186 - protein, 10 aa, respiratory syncytial virus
YIDKQLLPIV

SEQ ID NO: 187 - protein, 10 aa, respiratory syncytial virus
IDKQLLPIVN

SEQ ID NO: 188 - protein, 10 aa, respiratory syncytial virus
DKQLLPIVNK

SEQ ID NO: 189 - protein, 10 aa, respiratory syncytial virus
KQLLPIVNKQ

SEQ ID NO: 190 - protein, 10 aa, respiratory syncytial virus
QLLPIVNKQS

SEQ ID NO: 191 - protein, 10 aa, respiratory syncytial virus
LLPIVNKQSC

SEQ ID NO: 192 - protein, 10 aa, respiratory syncytial virus
LPIVNKQSCS

SEQ ID NO: 193 - protein, 10 aa, respiratory syncytial virus
PIVNKQSCSI

SEQ ID NO: 194 - protein, 10 aa, respiratory syncytial virus
IVNKQSCSIS

SEQ ID NO: 195 - protein, 10 aa, respiratory syncytial virus
VNKQSCSISN

SEQ ID NO: 196 - protein, 10 aa, respiratory syncytial virus
NKQSCSISNI

SEQ ID NO: 197 - protein, 10 aa, respiratory syncytial virus
KQSCSISNIE

SEQ ID NO: 198 - protein, 10 aa, respiratory syncytial virus
QSCSISNIET

SEQ ID NO: 199 - protein, 10 aa, respiratory syncytial virus
SCSISNIETV

SEQ ID NO: 200 - protein, 10 aa, respiratory syncytial virus
CSISNIETVI

SEQ ID NO: 201 - protein, 10 aa, respiratory syncytial virus
SISNIETVIE

SEQ ID NO: 202 - protein, 10 aa, respiratory syncytial virus
ISNIETVIEF

SEQ ID NO: 203 - protein, 10 aa, respiratory syncytial virus
SNIETVIEFQ

SEQ ID NO: 204 - protein, 10 aa, respiratory syncytial virus
NIETVIEFQQ

SEQ ID NO: 205 - protein, 10 aa, respiratory syncytial virus
IETVIEFQQK

SEQ ID NO: 206 - protein, 10 aa, respiratory syncytial virus
ETVIEFQQKN

SEQ ID NO: 207 - protein, 10 aa, respiratory syncytial virus
TVIEFQQKNN

SEQ ID NO: 208 - protein, 10 aa, respiratory syncytial virus
VIEFQQKNNR

SEQ ID NO: 209 - protein, 10 aa, respiratory syncytial virus
IEFQQKNNRL

SEQ ID NO: 210 - protein, 10 aa, respiratory syncytial virus
EFQQKNNRLL

SEQ ID NO: 211 - protein, 10 aa, respiratory syncytial virus
FQQKNNRLLE

SEQ ID NO: 212 - protein, 10 aa, respiratory syncytial virus
QQKNNRLLEI

SEQ ID NO: 213 - protein, 10 aa, respiratory syncytial virus
QKNNRLLEIT

SEQ ID NO: 214 - protein, 10 aa, respiratory syncytial virus
KNNRLLEITR

SEQ ID NO: 215 - protein, 10 aa, respiratory syncytial virus
NNRLLEITRE

SEQ ID NO: 216 - protein, 10 aa, respiratory syncytial virus
NRLLEITREF

SEQ ID NO: 217 - protein, 10 aa, respiratory syncytial virus
RLLEITREFS

SEQ ID NO: 218 - protein, 10 aa, respiratory syncytial virus
LLEITREFSV

SEQ ID NO: 219 - protein, 10 aa, respiratory syncytial virus
LEITREFSVN

SEQ ID NO: 220 - protein, 10 aa, respiratory syncytial virus
EITREFSVNA

SEQ ID NO: 221 - protein, 10 aa, respiratory syncytial virus
ITREFSVNAG

SEQ ID NO: 222 - protein, 10 aa, respiratory syncytial virus
TREFSVNAGV

SEQ ID NO: 223 - protein, 10 aa, respiratory syncytial virus
REFSVNAGVT

SEQ ID NO: 224 - protein, 10 aa, respiratory syncytial virus
EFSVNAGVTT

SEQ ID NO: 225 - protein, 10 aa, respiratory syncytial virus
FSVNAGVTTP

SEQ ID NO: 226 - protein, 10 aa, respiratory syncytial virus
SVNAGVTTPV

SEQ ID NO: 227 - protein, 10 aa, respiratory syncytial virus
VNAGVTTPVS

SEQ ID NO: 228 - protein, 10 aa, respiratory syncytial virus
NAGVTTPVST

SEQ ID NO: 229 - protein, 10 aa, respiratory syncytial virus
AGVTTPVSTY

SEQ ID NO: 230 - protein, 10 aa, respiratory syncytial virus
GVTTPVSTYM

SEQ ID NO: 231 - protein, 10 aa, respiratory syncytial virus
VTTPVSTYML

SEQ ID NO: 232 - protein, 10 aa, respiratory syncytial virus
TTPVSTYMLT

SEQ ID NO: 233 - protein, 10 aa, respiratory syncytial virus
TPVSTYMLTN

SEQ ID NO: 234 - protein, 10 aa, respiratory syncytial virus
PVSTYMLTNS

SEQ ID NO: 235 - protein, 10 aa, respiratory syncytial virus
VSTYMLTNSE

SEQ ID NO: 236 - protein, 10 aa, respiratory syncytial virus
STYMLTNSEL

SEQ ID NO: 237 - protein, 10 aa, respiratory syncytial virus
TYMLTNSELL

SEQ ID NO: 238 - protein, 10 aa, respiratory syncytial virus
YMLTNSELLS

SEQ ID NO: 239 - protein, 10 aa, respiratory syncytial virus
MLTNSELLSL

SEQ ID NO: 240 - protein, 10 aa, respiratory syncytial virus
LTNSELLSLI

SEQ ID NO: 241 - protein, 10 aa, respiratory syncytial virus
TNSELLSLIN

SEQ ID NO: 242 - protein, 10 aa, respiratory syncytial virus
NSELLSLIND

SEQ ID NO: 243 - protein, 10 aa, respiratory syncytial virus
SELLSLINDM

SEQ ID NO: 244 - protein, 10 aa, respiratory syncytial virus
ELLSLINDMP

SEQ ID NO: 245 - protein, 10 aa, respiratory syncytial virus
LLSLINDMPI

SEQ ID NO: 246 - protein, 10 aa, respiratory syncytial virus
LSLINDMPIT

SEQ ID NO: 247 - protein, 10 aa, respiratory syncytial virus
SLINDMPITN

SEQ ID NO: 248 - protein, 10 aa, respiratory syncytial virus
LINDMPITND

SEQ ID NO: 249 - protein, 10 aa, respiratory syncytial virus
INDMPITNDQ

SEQ ID NO: 250 - protein, 10 aa, respiratory syncytial virus
NDMPITNDQK

SEQ ID NO: 251 - protein, 10 aa, respiratory syncytial virus
DMPITNDQKK

SEQ ID NO: 252 - protein, 10 aa, respiratory syncytial virus
MPITNDQKKL

SEQ ID NO: 253 - protein, 10 aa, respiratory syncytial virus
PITNDQKKLM

SEQ ID NO: 254 - protein, 10 aa, respiratory syncytial virus
ITNDQKKLMS

SEQ ID NO: 255 - protein, 10 aa, respiratory syncytial virus
TNDQKKLMSN

SEQ ID NO: 256 - protein, 10 aa, respiratory syncytial virus
NDQKKLMSNN

SEQ ID NO: 257 - protein, 10 aa, respiratory syncytial virus
DQKKLMSNNV

SEQ ID NO: 258 - protein, 10 aa, respiratory syncytial virus
QKKLMSNNVQ

SEQ ID NO: 259 - protein, 10 aa, respiratory syncytial virus
KKLMSNNVQI

SEQ ID NO: 260 - protein, 10 aa, respiratory syncytial virus
KLMSNNVQIV

SEQ ID NO: 261 - protein, 10 aa, respiratory syncytial virus
LMSNNVQIVR

SEQ ID NO: 262 - protein, 10 aa, respiratory syncytial virus
MSNNVQIVRQ

SEQ ID NO: 263 - protein, 10 aa, respiratory syncytial virus
SNNVQIVRQQ

SEQ ID NO: 264 - protein, 10 aa, respiratory syncytial virus
NNVQIVRQQS

SEQ ID NO: 265 - protein, 10 aa, respiratory syncytial virus
NVQIVRQQSY

SEQ ID NO: 266 - protein, 10 aa, respiratory syncytial virus
VQIVRQQSYS

SEQ ID NO: 267 - protein, 10 aa, respiratory syncytial virus
QIVRQQSYSI

SEQ ID NO: 268 - protein, 10 aa, respiratory syncytial virus
IVRQQSYSIM

SEQ ID NO: 269 - protein, 10 aa, respiratory syncytial virus
VRQQSYSIMS

SEQ ID NO: 270 - protein, 10 aa, respiratory syncytial virus
RQQSYSIMSI

SEQ ID NO: 271 - protein, 10 aa, respiratory syncytial virus
QQSYSIMSII

SEQ ID NO: 272 - protein, 10 aa, respiratory syncytial virus
SYSIMSIIKE

SEQ ID NO: 273 - protein, 10 aa, respiratory syncytial virus
SYSIMSIIKE

SEQ ID NO: 274 - protein, 10 aa, respiratory syncytial virus
YSIMSIIKEE

SEQ ID NO: 275 - protein, 10 aa, respiratory syncytial virus
SIMSIIKEEV

SEQ ID NO: 276 - protein, 10 aa, respiratory syncytial virus
IMSIIKEEVL

SEQ ID NO: 277 - protein, 10 aa, respiratory syncytial virus
MSIIKEEVLA

SEQ ID NO: 278 - protein, 10 aa, respiratory syncytial virus
SIIKEEVLAY

SEQ ID NO: 279 - protein, 10 aa, respiratory syncytial virus
IIKEEVLAYV

SEQ ID NO: 280 - protein, 10 aa, respiratory syncytial virus
IKEEVLAYVV

SEQ ID NO: 281 - protein, 10 aa, respiratory syncytial virus
KEEVLAYVVQ

SEQ ID NO: 282 - protein, 10 aa, respiratory syncytial virus
EEVLAYVVQL

SEQ ID NO: 283 - protein, 10 aa, respiratory syncytial virus
EVLAYVVQLP

SEQ ID NO: 284 - protein, 10 aa, respiratory syncytial virus
VLAYVVQLPL

SEQ ID NO: 285 - protein, 10 aa, respiratory syncytial virus
LAYVVQLPLY

SEQ ID NO: 286 - protein, 10 aa, respiratory syncytial virus
AYVVQLPLYG

SEQ ID NO: 287 - protein, 10 aa, respiratory syncytial virus
YVVQLPLYGV

SEQ ID NO: 288 - protein, 10 aa, respiratory syncytial virus
VVQLPLYGVI

SEQ ID NO: 289 - protein, 10 aa, respiratory syncytial virus
VQLPLYGVID

SEQ ID NO: 290 - protein, 10 aa, respiratory syncytial virus
QLPLYGVIDT

SEQ ID NO: 291 - protein, 10 aa, respiratory syncytial virus
LPLYGVIDTP

SEQ ID NO: 292 - protein, 10 aa, respiratory syncytial virus
PLYGVIDTPC

SEQ ID NO: 293 - protein, 10 aa, respiratory syncytial virus
LYGVIDTPCW

SEQ ID NO: 294 - protein, 10 aa, respiratory syncytial virus
YGVIDTPCWK

SEQ ID NO: 295 - protein, 10 aa, respiratory syncytial virus
GVIDTPCWKL

SEQ ID NO: 296 - protein, 10 aa, respiratory syncytial virus
VIDTPCWKLH

SEQ ID NO: 297 - protein, 10 aa, respiratory syncytial virus
IDTPCWKLHT

SEQ ID NO: 298 - protein, 10 aa, respiratory syncytial virus
DTPCWKLHTS

SEQ ID NO: 299 - protein, 10 aa, respiratory syncytial virus
TPCWKLHTSP

SEQ ID NO: 300 - protein, 10 aa, respiratory syncytial virus
PCWKLHTSPL

SEQ ID NO: 301 - protein, 10 aa, respiratory syncytial virus
CWKLHTSPLC

SEQ ID NO: 302 - protein, 10 aa, respiratory syncytial virus
WKLHTSPLCT

SEQ ID NO: 303 - protein, 10 aa, respiratory syncytial virus
KLHTSPLCTT

SEQ ID NO: 304 - protein, 10 aa, respiratory syncytial virus
LHTSPLCTTN

SEQ ID NO: 305 - protein, 10 aa, respiratory syncytial virus
HTSPLCTTNT

SEQ ID NO: 306 - protein, 10 aa, respiratory syncytial virus
TSPLCTTNTK

SEQ ID NO: 307 - protein, 10 aa, respiratory syncytial virus
SPLCTTNTKE

SEQ ID NO: 308 - protein, 10 aa, respiratory syncytial virus
PLCTTNTKEG

SEQ ID NO: 309 - protein, 10 aa, respiratory syncytial virus
LCTTNTKEGS

SEQ ID NO: 310 - protein, 10 aa, respiratory syncytial virus
CTTNTKEGSN

SEQ ID NO: 311 - protein, 10 aa, respiratory syncytial virus
TTNTKEGSNI

SEQ ID NO: 312 - protein, 10 aa, respiratory syncytial virus
TNTKEGSNIC

SEQ ID NO: 313 - protein, 10 aa, respiratory syncytial virus
NTKEGSNICL

SEQ ID NO: 314 - protein, 10 aa, respiratory syncytial virus
TKEGSNICLT

SEQ ID NO: 315 - protein, 10 aa, respiratory syncytial virus
KEGSNICLTR

SEQ ID NO: 316 - protein, 10 aa, respiratory syncytial virus
EGSNICLTRT

SEQ ID NO: 317 - protein, 10 aa, respiratory syncytial virus
GSNICLTRTD

SEQ ID NO: 318 - protein, 10 aa, respiratory syncytial virus
SNICLTRTDR

SEQ ID NO: 319 - protein, 10 aa, respiratory syncytial virus
NICLTRTDRG

SEQ ID NO: 320 - protein, 10 aa, respiratory syncytial virus
ICLTRTDRGW

SEQ ID NO: 321 - protein, 10 aa, respiratory syncytial virus
CLTRTDRGWY

SEQ ID NO: 322 - protein, 10 aa, respiratory syncytial virus
LTRTDRGWYC

SEQ ID NO: 323 - protein, 10 aa, respiratory syncytial virus
TRTDRGWYCD

SEQ ID NO: 324 - protein, 10 aa, respiratory syncytial virus
RTDRGWYCDN

SEQ ID NO: 325 - protein, 10 aa, respiratory syncytial virus
TDRGWYCDNA

SEQ ID NO: 326 - protein, 10 aa, respiratory syncytial virus
DRGWYCDNAG

SEQ ID NO: 327 - protein, 10 aa, respiratory syncytial virus
RGWYCDNAGS

SEQ ID NO: 328 - protein, 10 aa, respiratory syncytial virus
GWYCDNAGSV

SEQ ID NO: 329 - protein, 10 aa, respiratory syncytial virus
WYCDNAGSVS

SEQ ID NO: 330 - protein, 10 aa, respiratory syncytial virus
YCDNAGSVSF

SEQ ID NO: 331 - protein, 10 aa, respiratory syncytial virus
CDNAGSVSFF

SEQ ID NO: 332 - protein, 10 aa, respiratory syncytial virus
DNAGSVSFFP

SEQ ID NO: 333 - protein, 10 aa, respiratory syncytial virus
NAGSVSFFPQ

SEQ ID NO: 334 - protein, 10 aa, respiratory syncytial virus
AGSVSFFPQA

SEQ ID NO: 335 - protein, 10 aa, respiratory syncytial virus
GSVSFFPQAE

SEQ ID NO: 336 - protein, 10 aa, respiratory syncytial virus
SVSFFPQAET

SEQ ID NO: 337 - protein, 10 aa, respiratory syncytial virus
VSFFPQAETC

SEQ ID NO: 338 - protein, 10 aa, respiratory syncytial virus
SFFPQAETCK

SEQ ID NO: 339 - protein, 10 aa, respiratory syncytial virus
FFPQAETCKV

SEQ ID NO: 340 - protein, 10 aa, respiratory syncytial virus
FPQAETCKVQ

SEQ ID NO: 341 - protein, 10 aa, respiratory syncytial virus
PQAETCKVQS

SEQ ID NO: 342 - protein, 10 aa, respiratory syncytial virus
QAETCKVQSN

SEQ ID NO: 343 - protein, 10 aa, respiratory syncytial virus
AETCKVQSNR

SEQ ID NO: 344 - protein, 10 aa, respiratory syncytial virus
ETCKVQSNRV

SEQ ID NO: 345 - protein, 10 aa, respiratory syncytial virus
TCKVQSNRVF

SEQ ID NO: 346 - protein, 10 aa, respiratory syncytial virus
CKVQSNRVFC

SEQ ID NO: 347 - protein, 10 aa, respiratory syncytial virus
KVQSNRVFCD

SEQ ID NO: 348 - protein, 10 aa, respiratory syncytial virus
VQSNRVFCDT

SEQ ID NO: 349 - protein, 10 aa, respiratory syncytial virus
QSNRVFCDTM

SEQ ID NO: 350 - protein, 10 aa, respiratory syncytial virus
SNRVFCDTMN

SEQ ID NO: 351 - protein, 10 aa, respiratory syncytial virus
NRVFCDTMNS

SEQ ID NO: 352 - protein, 10 aa, respiratory syncytial virus
RVFCDTMNSL

SEQ ID NO: 353 - protein, 10 aa, respiratory syncytial virus
VFCDTMNSLT

SEQ ID NO: 354 - protein, 10 aa, respiratory syncytial virus
FCDTMNSLTL

SEQ ID NO: 355 - protein, 10 aa, respiratory syncytial virus
CDTMNSLTLP

SEQ ID NO: 356 - protein, 10 aa, respiratory syncytial virus
DTMNSLTLPS

SEQ ID NO: 357 - protein, 10 aa, respiratory syncytial virus
TMNSLTLPSE

SEQ ID NO: 358 - protein, 10 aa, respiratory syncytial virus
MNSLTLPSEV

SEQ ID NO: 359 - protein, 10 aa, respiratory syncytial virus
NSLTLPSEVN

SEQ ID NO: 360 - protein, 10 aa, respiratory syncytial virus
SLTLPSEVNL

SEQ ID NO: 361 - protein, 10 aa, respiratory syncytial virus
LTLPSEVNLC

SEQ ID NO: 362 - protein, 10 aa, respiratory syncytial virus
TLPSEVNLCN

SEQ ID NO: 363 - protein, 10 aa, respiratory syncytial virus
LPSEVNLCNV

SEQ ID NO: 364 - protein, 10 aa, respiratory syncytial virus
PSEVNLCNVD

SEQ ID NO: 365 - protein, 10 aa, respiratory syncytial virus
SEVNLCNVDI

SEQ ID NO: 366 - protein, 10 aa, respiratory syncytial virus
EVNLCNVDIF

SEQ ID NO: 367 - protein, 10 aa, respiratory syncytial virus
VNLCNVDIFN

SEQ ID NO: 368 - protein, 10 aa, respiratory syncytial virus
NLCNVDIFNP

SEQ ID NO: 369 - protein, 10 aa, respiratory syncytial virus
LCNVDIFNPK

SEQ ID NO: 370 - protein, 10 aa, respiratory syncytial virus
CNVDIFNPKY

SEQ ID NO: 371 - protein, 10 aa, respiratory syncytial virus
NVDIFNPKYD

SEQ ID NO: 372 - protein, 10 aa, respiratory syncytial virus
VDIFNPKYDC

SEQ ID NO: 373 - protein, 10 aa, respiratory syncytial virus
DIFNPKYDCK

SEQ ID NO: 374 - protein, 10 aa, respiratory syncytial virus
IFNPKYDCKI

SEQ ID NO: 375 - protein, 10 aa, respiratory syncytial virus
FNPKYDCKIM

SEQ ID NO: 376 - protein, 10 aa, respiratory syncytial virus
NPKYDCKIMT

SEQ ID NO: 377 - protein, 10 aa, respiratory syncytial virus
PKYDCKIMTS

SEQ ID NO: 378 - protein, 10 aa, respiratory syncytial virus
KYDCKIMTSK

SEQ ID NO: 379 - protein, 10 aa, respiratory syncytial virus
YDCKIMTSKT

SEQ ID NO: 380 - protein, 10 aa, respiratory syncytial virus
DCKIMTSKTD

SEQ ID NO: 381 - protein, 10 aa, respiratory syncytial virus
CKIMTSKTDV

SEQ ID NO: 382 - protein, 10 aa, respiratory syncytial virus
KIMTSKTDVS

SEQ ID NO: 383 - protein, 10 aa, respiratory syncytial virus
IMTSKTDVSS

SEQ ID NO: 384 - protein, 10 aa, respiratory syncytial virus
MTSKTDVSSS

SEQ ID NO: 385 - protein, 10 aa, respiratory syncytial virus
TSKTDVSSSV

SEQ ID NO: 386 - protein, 10 aa, respiratory syncytial virus
SKTDVSSSVI

SEQ ID NO: 387 - protein, 10 aa, respiratory syncytial virus
KTDVSSSVIT

SEQ ID NO: 388 - protein, 10 aa, respiratory syncytial virus
TDVSSSVITS

SEQ ID NO: 389 - protein, 10 aa, respiratory syncytial virus
DVSSSVITSL

SEQ ID NO: 390 - protein, 10 aa, respiratory syncytial virus
VSSSVITSLG

SEQ ID NO: 391 - protein, 10 aa, respiratory syncytial virus
SSSVITSLGA

SEQ ID NO: 392 - protein, 10 aa, respiratory syncytial virus
SSVITSLGAI

SEQ ID NO: 393 - protein, 10 aa, respiratory syncytial virus
SVITSLGAIV

SEQ ID NO: 394 - protein, 10 aa, respiratory syncytial virus
VITSLGAIVS

SEQ ID NO: 395 - protein, 10 aa, respiratory syncytial virus
ITSLGAIVSC

SEQ ID NO: 396 - protein, 10 aa, respiratory syncytial virus
TSLGAIVSCY

SEQ ID NO: 397 - protein, 10 aa, respiratory syncytial virus
SLGAIVSCYG

SEQ ID NO: 398 - protein, 10 aa, respiratory syncytial virus
LGAIVSCYGK

SEQ ID NO: 399 - protein, 10 aa, respiratory syncytial virus
GAIVSCYGKT

SEQ ID NO: 400 - protein, 10 aa, respiratory syncytial virus
AIVSCYGKTK

SEQ ID NO: 401 - protein, 10 aa, respiratory syncytial virus
IVSCYGKTKC

SEQ ID NO: 402 - protein, 10 aa, respiratory syncytial virus
VSCYGKTKCT

SEQ ID NO: 403 - protein, 10 aa, respiratory syncytial virus
SCYGKTKCTA

SEQ ID NO: 404 - protein, 10 aa, respiratory syncytial virus
CYGKTKCTAS

SEQ ID NO: 405 - protein, 10 aa, respiratory syncytial virus
YGKTKCTASN

SEQ ID NO: 406 - protein, 10 aa, respiratory syncytial virus
GKTKCTASNK

SEQ ID NO: 407 - protein, 10 aa, respiratory syncytial virus
KTKCTASNKN

SEQ ID NO: 408 - protein, 10 aa, respiratory syncytial virus
TKCTASNKNR

SEQ ID NO: 409 - protein, 10 aa, respiratory syncytial virus
KCTASNKNRG

SEQ ID NO: 410 - protein, 10 aa, respiratory syncytial virus
CTASNKNRGI

SEQ ID NO: 411 - protein, 10 aa, respiratory syncytial virus
TASNKNRGII

SEQ ID NO: 412 - protein, 10 aa, respiratory syncytial virus
ASNKNRGIIK

SEQ ID NO: 413 - protein, 10 aa, respiratory syncytial virus
SNKNRGIIKT

SEQ ID NO: 414 - protein, 10 aa, respiratory syncytial virus
NKNRGIIKTF

SEQ ID NO: 415 - protein, 10 aa, respiratory syncytial virus
KNRGIIKTFS

SEQ ID NO: 416 - protein, 10 aa, respiratory syncytial virus
NRGIIKTFSN

SEQ ID NO: 417 - protein, 10 aa, respiratory syncytial virus
RGIIKTFSNG

SEQ ID NO: 418 - protein, 10 aa, respiratory syncytial virus
GIIKTFSNGC

SEQ ID NO: 419 - protein, 10 aa, respiratory syncytial virus
IIKTFSNGCD

SEQ ID NO: 420 - protein, 10 aa, respiratory syncytial virus
IKTFSNGCDY

SEQ ID NO: 421 - protein, 10 aa, respiratory syncytial virus
KTFSNGCDYV

SEQ ID NO: 422 - protein, 10 aa, respiratory syncytial virus
TFSNGCDYVS

SEQ ID NO: 423 - protein, 10 aa, respiratory syncytial virus
FSNGCDYVSN

SEQ ID NO: 424 - protein, 10 aa, respiratory syncytial virus
SNGCDYVSNK

SEQ ID NO: 425 - protein, 10 aa, respiratory syncytial virus
NGCDYVSNKG

SEQ ID NO: 426 - protein, 10 aa, respiratory syncytial virus
GCDYVSNKGV

SEQ ID NO: 427 - protein, 10 aa, respiratory syncytial virus
CDYVSNKGVD

SEQ ID NO: 428 - protein, 10 aa, respiratory syncytial virus
DYVSNKGVDT

SEQ ID NO: 429 - protein, 10 aa, respiratory syncytial virus
YVSNKGVDTV

SEQ ID NO: 430 - protein, 10 aa, respiratory syncytial virus
VSNKGVDTVS

SEQ ID NO: 431 - protein, 10 aa, respiratory syncytial virus
SNKGVDTVSV

SEQ ID NO: 432 - protein, 10 aa, respiratory syncytial virus
NKGVDTVSVG

SEQ ID NO: 433 - protein, 10 aa, respiratory syncytial virus
KGVDTVSVGN

SEQ ID NO: 434 - protein, 10 aa, respiratory syncytial virus
GVDTVSVGNT

SEQ ID NO: 435 - protein, 10 aa, respiratory syncytial virus
VDTVSVGNTL

SEQ ID NO: 436 - protein, 10 aa, respiratory syncytial virus
DTVSVGNTLY

SEQ ID NO: 437 - protein, 10 aa, respiratory syncytial virus
TVSVGNTLYY

SEQ ID NO: 438 - protein, 10 aa, respiratory syncytial virus
VSVGNTLYYV

SEQ ID NO: 439 - protein, 10 aa, respiratory syncytial virus
SVGNTLYYVN

SEQ ID NO: 440 - protein, 10 aa, respiratory syncytial virus
VGNTLYYVNK

SEQ ID NO: 441 - protein, 10 aa, respiratory syncytial virus
GNTLYYVNKQ

SEQ ID NO: 442 - protein, 10 aa, respiratory syncytial virus
NTLYYVNKQE

SEQ ID NO: 443 - protein, 10 aa, respiratory syncytial virus
TLYYVNKQEG

SEQ ID NO: 444 - protein, 10 aa, respiratory syncytial virus
LYYVNKQEGK

SEQ ID NO: 445 - protein, 10 aa, respiratory syncytial virus
YYVNKQEGKS

SEQ ID NO: 446 - protein, 10 aa, respiratory syncytial virus
YVNKQEGKSL

SEQ ID NO: 447 - protein, 10 aa, respiratory syncytial virus
VNKQEGKSLY

SEQ ID NO: 448 - protein, 10 aa, respiratory syncytial virus
NKQEGKSLYV

SEQ ID NO: 449 - protein, 10 aa, respiratory syncytial virus
KQEGKSLYVK

SEQ ID NO: 450 - protein, 10 aa, respiratory syncytial virus
QEGKSLYVKG

SEQ ID NO: 451 - protein, 10 aa, respiratory syncytial virus
EGKSLYVKGE

SEQ ID NO: 452 - protein, 10 aa, respiratory syncytial virus
GKSLYVKGEP

SEQ ID NO: 453 - protein, 10 aa, respiratory syncytial virus
KSLYVKGEPI

SEQ ID NO: 454 - protein, 10 aa, respiratory syncytial virus
SLYVKGEPII

SEQ ID NO: 455 - protein, 10 aa, respiratory syncytial virus
LYVKGEPIIN

SEQ ID NO: 456 - protein, 10 aa, respiratory syncytial virus
YVKGEPIINF

SEQ ID NO: 457 - protein, 10 aa, respiratory syncytial virus
VKGEPIINFY

SEQ ID NO: 458 - protein, 10 aa, respiratory syncytial virus
KGEPIINFYD

SEQ ID NO: 459 - protein, 10 aa, respiratory syncytial virus
GEPIINFYDP

SEQ ID NO: 460 - protein, 10 aa, respiratory syncytial virus
EPIINFYDPL

SEQ ID NO: 461 - protein, 10 aa, respiratory syncytial virus
PIINFYDPLV

SEQ ID NO: 462 - protein, 10 aa, respiratory syncytial virus
IINFYDPLVF

SEQ ID NO: 463 - protein, 10 aa, respiratory syncytial virus
INFYDPLVFP

SEQ ID NO: 464 - protein, 10 aa, respiratory syncytial virus
NFYDPLVFPS

SEQ ID NO: 465 - protein, 10 aa, respiratory syncytial virus
FYDPLVFPSD

SEQ ID NO: 466 - protein, 10 aa, respiratory syncytial virus
YDPLVFPSDE

SEQ ID NO: 467 - protein, 10 aa, respiratory syncytial virus
DPLVFPSDEF

SEQ ID NO: 468 - protein, 10 aa, respiratory syncytial virus
PLVFPSDEFD

SEQ ID NO: 469 - protein, 10 aa, respiratory syncytial virus
LVFPSDEFDA

SEQ ID NO: 470 - protein, 10 aa, respiratory syncytial virus
VFPSDEFDAS

SEQ ID NO: 471 - protein, 10 aa, respiratory syncytial virus
FPSDEFDASI

SEQ ID NO: 472 - protein, 10 aa, respiratory syncytial virus
PSDEFDASIS

SEQ ID NO: 473 - protein, 10 aa, respiratory syncytial virus
SDEFDASISQ

SEQ ID NO: 474 - protein, 10 aa, respiratory syncytial virus
DEFDASISQV

SEQ ID NO: 475 - protein, 10 aa, respiratory syncytial virus
EFDASISQVN

SEQ ID NO: 476 - protein, 10 aa, respiratory syncytial virus
FDASISQVNE

SEQ ID NO: 477 - protein, 10 aa, respiratory syncytial virus
DASISQVNEK

SEQ ID NO: 478 - protein, 10 aa, respiratory syncytial virus
ASISQVNEKI

SEQ ID NO: 479 - protein, 10 aa, respiratory syncytial virus
SISQVNEKIN

SEQ ID NO: 480 - protein, 10 aa, respiratory syncytial virus
ISQVNEKINQ

SEQ ID NO: 481 - protein, 10 aa, respiratory syncytial virus
SQVNEKINQS

SEQ ID NO: 482 - protein, 10 aa, respiratory syncytial virus
QVNEKINQSL

SEQ ID NO: 483 - protein, 10 aa, respiratory syncytial virus
VNEKINQSLA

SEQ ID NO: 484 - protein, 10 aa, respiratory syncytial virus
NEKINQSLAF

SEQ ID NO: 485 - protein, 10 aa, respiratory syncytial virus
EKINQSLAFI

SEQ ID NO: 486 - protein, 10 aa, respiratory syncytial virus
KINQSLAFIR

SEQ ID NO: 487 - protein, 10 aa, respiratory syncytial virus
INQSLAFIRK

SEQ ID NO: 488 - protein, 10 aa, respiratory syncytial virus
NQSLAFIRKS

SEQ ID NO: 489 - protein, 10 aa, respiratory syncytial virus
QSLAFIRKSD

SEQ ID NO: 490 - protein, 10 aa, respiratory syncytial virus
SLAFIRKSDE

SEQ ID NO: 491 - protein, 10 aa, respiratory syncytial virus
LAFIRKSDEL

SEQ ID NO: 492 - protein, 10 aa, respiratory syncytial virus
AFIRKSDELL

SEQ ID NO: 493 - protein, 10 aa, respiratory syncytial virus
FIRKSDELLH

SEQ ID NO: 494 - protein, 10 aa, respiratory syncytial virus
IRKSDELLHN

SEQ ID NO: 495 - protein, 10 aa, respiratory syncytial virus
RKSDELLHNV

SEQ ID NO: 496 - protein, 10 aa, respiratory syncytial virus
KSDELLHNVN

SEQ ID NO: 497 - protein, 10 aa, respiratory syncytial virus
SDELLHNVNA

SEQ ID NO: 498 - protein, 10 aa, respiratory syncytial virus
DELLHNVNAG

SEQ ID NO: 499 - protein, 10 aa, respiratory syncytial virus
ELLHNVNAGK

SEQ ID NO: 500 - protein, 10 aa, respiratory syncytial virus
LLHNVNAGKS

SEQ ID NO: 501 - protein, 10 aa, respiratory syncytial virus
LHNVNAGKST

SEQ ID NO: 502 - protein, 10 aa, respiratory syncytial virus
HNVNAGKSTT

SEQ ID NO: 503 - protein, 10 aa, respiratory syncytial virus
NVNAGKSTTN

SEQ ID NO: 504 - protein, 10 aa, respiratory syncytial virus
VNAGKSTTNI

SEQ ID NO: 505 - protein, 10 aa, respiratory syncytial virus
NAGKSTTNIM

SEQ ID NO: 506 - protein, 10 aa, respiratory syncytial virus
AGKSTTNIMI

SEQ ID NO: 507 - protein, 10 aa, respiratory syncytial virus
GKSTTNIMIT

SEQ ID NO: 508 - protein, 10 aa, respiratory syncytial virus
KSTTNIMITT

SEQ ID NO: 509 - protein, 10 aa, respiratory syncytial virus
STTNIMITTI

SEQ ID NO: 510 - protein, 10 aa, respiratory syncytial virus
TTNIMITTII

SEQ ID NO: 511 - protein, 10 aa, respiratory syncytial virus
TNIMITTIII

SEQ ID NO: 512 - protein, 10 aa, respiratory syncytial virus
NIMITTIIIV

SEQ ID NO: 513 - protein, 10 aa, respiratory syncytial virus
IMITTIIIVI

SEQ ID NO: 514 - protein, 10 aa, respiratory syncytial virus
MITTIIIVII

SEQ ID NO: 515 - protein, 10 aa, respiratory syncytial virus
ITTIIIVIIV

SEQ ID NO: 516 - protein, 10 aa, respiratory syncytial virus
TTIIIVIIVI

SEQ ID NO: 517 - protein, 10 aa, respiratory syncytial virus
TIIIVIIVIL

SEQ ID NO: 518 - protein, 10 aa, respiratory syncytial virus
IIIVIIVILL

SEQ ID NO: 519 - protein, 10 aa, respiratory syncytial virus
IIVIIVILLS

SEQ ID NO: 520 - protein, 10 aa, respiratory syncytial virus
IVIIVILLSL

SEQ ID NO: 521 - protein, 10 aa, respiratory syncytial virus
VIIVILLSLI

SEQ ID NO: 522 - protein, 10 aa, respiratory syncytial virus
IIVILLSLIA

SEQ ID NO: 523 - protein, 10 aa, respiratory syncytial virus
IVILLSLIAV

SEQ ID NO: 524 - protein, 10 aa, respiratory syncytial virus
VILLSLIAVG

SEQ ID NO: 525 - protein, 10 aa, respiratory syncytial virus
ILLSLIAVGL

SEQ ID NO: 526 - protein, 10 aa, respiratory syncytial virus
LLSLIAVGLL

SEQ ID NO: 527 - protein, 10 aa, respiratory syncytial virus
LSLIAVGLLL

SEQ ID NO: 528 - protein, 10 aa, respiratory syncytial virus
SLIAVGLLLY

SEQ ID NO: 529 - protein, 10 aa, respiratory syncytial virus
LIAVGLLLYC

SEQ ID NO: 530 - protein, 10 aa, respiratory syncytial virus
IAVGLLLYCK

SEQ ID NO: 531 - protein, 10 aa, respiratory syncytial virus
AVGLLLYCKA

SEQ ID NO: 532 - protein, 10 aa, respiratory syncytial virus
VGLLLYCKAR

SEQ ID NO: 533 - protein, 10 aa, respiratory syncytial virus
GLLLYCKARS

SEQ ID NO: 534 - protein, 10 aa, respiratory syncytial virus
LLLYCKARST

SEQ ID NO: 535 - protein, 10 aa, respiratory syncytial virus
LLYCKARSTP

SEQ ID NO: 536 - protein, 10 aa, respiratory syncytial virus
LYCKARSTPV

SEQ ID NO: 537 - protein, 10 aa, respiratory syncytial virus
YCKARSTPVT

SEQ ID NO: 538 - protein, 10 aa, respiratory syncytial virus
CKARSTPVTL

SEQ ID NO: 539 - protein, 10 aa, respiratory syncytial virus
KARSTPVTLS

SEQ ID NO: 540 - protein, 10 aa, respiratory syncytial virus
ARSTPVTLSK

SEQ ID NO: 541 - protein, 10 aa, respiratory syncytial virus
RSTPVTLSKD

SEQ ID NO: 542 - protein, 10 aa, respiratory syncytial virus
STPVTLSKDQ

SEQ ID NO: 543 - protein, 10 aa, respiratory syncytial virus
TPVTLSKDQL

SEQ ID NO: 544 - protein, 10 aa, respiratory syncytial virus
PVTLSKDQLS

SEQ ID NO: 545 - protein, 10 aa, respiratory syncytial virus
VTLSKDQLSG

SEQ ID NO: 546 - protein, 10 aa, respiratory syncytial virus
TLSKDQLSGI

SEQ ID NO: 547 - protein, 10 aa, respiratory syncytial virus
LSKDQLSGIN

SEQ ID NO: 548 - protein, 10 aa, respiratory syncytial virus
SKDQLSGINN

SEQ ID NO: 549 - protein, 10 aa, respiratory syncytial virus
KDQLSGINNI

SEQ ID NO: 550 - protein, 10 aa, respiratory syncytial virus
DQLSGINNIA

SEQ ID NO: 551 - protein, 10 aa, respiratory syncytial virus
QLSGINNIAF

SEQ ID NO: 552 - protein, 10 aa, respiratory syncytial virus
LSGINNIAFS

SEQ ID NO: 553 - protein, 10 aa, respiratory syncytial virus
SGINNIAFSN

SEQ ID NO: 554 - protein, 241 aa, respiratory syncytial virus
MEKFAPEFHGEDANNRATKFLESIKGKFTSPKDPKKKDSIISVNSIDIEVTKESPITSNSTIINPTNETDDTAGNKPNYQRKPLVSFKEDPTPSDNPFSKLYKETIETFDNNEEESSYSYEEINDQTNDNITARLDRIDEKLSEILGMLHTLVVASAGPTSARDGIRDAMIGLREEMIEKIRTEALMTNDRLEAMARLRNEESEKMAKDTSDEVSLNPTSEKLNNLLEGNDSDNDLSLEDF

SEQ ID NO: 555 - DNA, 726 bp, respiratory syncytial virus
atggaaaagtttgctcctgaattccatggagaagatgcaaacaacagggctactaaattcctagaatcaataaagggcaaattcacatcacccaaagatcccaagaaaaaagatagtatcatatctgtcaactcaatagatatagaagtaaccaaagaaagccctataacatcaaattcaactattatcaacccaacaaatgagacagatgatactgcagggaacaagcccaattatcaaagaaaacctctagtaagtttcaaagaagaccctacaccaagtgataatcccttttctaaactatacaaagaaaccatagaaacatttgataacaatgaagaagaatccagctattcatacgaagaaataaatgatcagacaaacgataatataacagcaagattagataggattgatgaaaaattaagtgaaatactaggaatgcttcacacattagtagtggcaagtgcaggacctacatctgctcgggatggtataagagatgccatgattggtttaagagaagaaatgatagaaaaaatcagaactgaagcattaatgaccaatgacagattagaagctatggcaagactcaggaatgaggaaagtgaaaagatggcaaaagacacatcagatgaagtgtctctcaatccaacatcagagaaattgaacaacctattggaagggaatgatagtgacaatgatctatcacttgaagatttctga

SEQ ID NO: 556 - DNA, 726 bp, Artificial Sequence
  Optimised Sequence
atggagaagttcgcccccgagttccacggcgaggacgccaacaatcgggccaccaagttcctggagagcatcaagggcaagttcaccagccccaaggaccccaagaaaaaggacagcatcatttccgtgaacagcatcgacatcgaggtgaccaaggagagccccatcacctccaacagcaccatcattaaccccaccaacgagacagacgataccgccggcaacaagcccaactaccagcggaagcccctggtgagcttcaaggaggaccccacccctagcgacaaccccttcagcaagctgtacaaggagaccatcgagaccttcgacaacaatgaggaagagagctcctacagctacgaagagatcaacgaccagaccaacgacaacatcaccgcccggctggacagaatcgacgagaagctgagcgagatcctgggcatgctgcacaccctggtggtcgccagcgccggccccaccagcgcccgggacggcatcagagacgccatgatcggcctgcgggaggaaatgatcgagaagatccggaccgaggccctgatgaccaacgaccggctggaggctatggccagactgcggaacgaggaaagcgagaagatggccaaggacaccagcgacgaggtgagcctgaaccccaccagcgagaagctgaacaatctgctcgagggcaacgacagcgataacgacctgagcctggaggacttctga

SEQ ID NO: 557 - protein, 391 aa, respiratory syncytial virus
MALSKVKLNDTLNKDQLLSSSKYTIQRSTGDSIDTPNYDVQKHINKLCGMLLITEDANHKFTGLIGMLYAMSRLGREDTIKILRDAGYHVKANGVDVTTHRQDINGKEMKFEVLTLASLTTEIQINIEIESRKSYKKMLKEMGEVAPEYRHDSPDCGMIILCIAALVITKLAAGDRSGLTAVIRRANNVLKNEMKRYKGLLPKDIANSFYEVFEKHPHFIDVFVHFGIAQSSTRGGSRVEGIFAGLFMNAYGAGQVMLRWGVLAKSVKNIMLGHASVQAEMEQVVEVYEYAQKLGGEAGFYHILNNPKASLLSLTQFPHFSSVVLGNAAGLGIMGEYRGTPRNQDLYDAAKAYAEQLKENGVINYSVLDLTAEELEAIKHQLNPKDNDVEL

SEQ ID NO: 558 - DNA, 1176 bp, respiratory syncytial virus
atggctcttagcaaagtcaagttgaatgatacactcaacaaagatcaacttctgtcatccagcaaatacaccatccaacggagcacaggagatagtattgatactcctaattatgatgtgcagaaacacatcaataagttatgtggcatgttattaatcacagaagatgctaatcataaattcactgggttaataggtatgttatatgcgatgtctaggttaggaagagaagacaccataaaaatactcagagatgcgggatatcatgtaaaagcaaatggagtagatgtaacaacacatcgtcaagacattaatggaaaagaaatgaaatttgaagtgttaacattggcaagcttaacaactgaaattcaaatcaacattgagatagaatctagaaaatcctacaaaaaaatgctaaaagaaatgggagaggtagctccagaatacaggcatgactctcctgattgtgggatgataatattatgtatagcagcattagtaataactaaattagcagcaggggacagatctggtcttacagccgtgattaggagagctaataatgtcctaaaaaatgaaatgaaacgttacaaaggcttactacccaaggacatagccaacagcttctatgaagtgtttgaaaaacatccccactttatagatgtttttgttcattttggtatagcacaatcttctaccagaggtggcagtagagttgaagggatttttgcaggattgtttatgaatgcctatggtgcagggcaagtgatgttacggtggggagtcttagcaaaatcagttaaaaatattatgttaggacatgctagtgtgcaagcagaaatggaacaagttgttgaggtttatgaatatgcccaaaaattgggtggtgaagcaggattctaccatatattgaacaacccaaaagcatcattattatctttgactcaatttcctcacttctccagtgtagtattaggcaatgctgctggcctaggcataatgggagagtacagaggtacaccgaggaatcaagatctatatgatgcagcaaaggcatatgctgaacaactcaaagaaaatggtgtgattaactacagtgtactagacttgacagcagaagaactagaggctatcaaacatcagcttaatccaaaagataatgatgtagagctttga

SEQ ID NO: 559 - DNA, 1176 bp, Artificial Sequence
  Optimised Sequence
atggccctgagcaaggtgaaactcaacgacaccctgaataaggatcagctcctgagctcctctaaatacaccatccagcggagcaccggcgacagcattgatacccccaactacgacgtgcagaagcacatcaacaaactgtgcggcatgctcctgatcaccgaggacgccaaccacaagttcaccggcctgatcgggatgctctacgccatgagccggctgggcagagaggacaccatcaagattctgcgggatgccggctaccacgtgaaggccaacggagtcgacgtgaccacacaccggcaggatatcaacggcaaggagatgaaattcgaagtgctgaccctcgccagcctgacaaccgagatccagattaacatcgaaattgagtcccggaagagctacaaaaagatgctgaaagagatgggcgaagtggcccccgagtaccggcacgacagccccgattgcggcatgatcattctgtgtatcgccgctctcgtgattaccaagctggccgctggcgaccggagcgggctgaccgccgtgatcagacgggctaacaatgtgctgaagaacgagatgaaacggtacaagggcctcctgcccaaagacatcgccaacagcttctacgaggtgtttgaaaagcacccccatttcatcgacgtctttgtgcacttcggcattgcccagagctccaccagaggcgggagccgggtggagggcatcttcgccgggctgtttatgaacgcttacggcgccgggcaggtgatgctgcggtggggcgtcctcgccaagagcgtgaaaaacatcatgctgggccacgccagcgtgcaggctgagatggaacaagtggtcgaggtgtacgaatatgcccagaagctgggcggagaggctggcttctaccacatcctgaacaatcccaaggccagcctcctgtccctcacccagtttccccacttcagctccgtcgtgctgggcaacgccgctggactcgggatcatgggcgagtaccggggaacccccagaaaccaggacctgtatgatgccgctaaggcatacgccgagcagctgaaagaaaacggcgtgatcaattacagcgtgctggacctcaccgccgaggaactggaggctatcaagcaccagctcaaccccaaagacaatgatgtggagctgtga

SEQ ID NO: 560 - protein, 64 aa, respiratory syncytial virus
MENTSITIEFSSKFWPYFTLIHMITTIISLLIIISIMIAILNKLCEYNVFHNKTFELPRARVNT

SEQ ID NO: 561 - DNA, 195 bp, respiratory syncytial virus
atggaaaatacatccataacaatagaattctcaagcaaattctggccttactttacactaatacacatgatcacaacaataatctctttgctaatcataatctccatcatgattgcaatactaaacaaactttgtgaatataacgtattccataacaaaacctttgagttaccaagagctcgagtcaacacatag

SEQ ID NO: 562 - DNA, 195 bp, Artificial Sequence
  Optimised sequence
atggagaacacctccatcaccattgagttctcctccaagttctggccatacttcaccctgatccacatgatcaccaccatcatctccctgctgatcatcatctccatcatgattgccatcctgaacaagctttgtgagtacaatgtcttccacaacaagacctttgagctgccccgggcccgggtgaacacctga

SEQ ID NO: 563 - protein, 6 aa, respiratory syncytial virus
KKRKRR

SEQ ID NO: 564 - protein, 4 aa, respiratory syncytial virus
RARR

SEQ ID NO: 565 - protein, 575 aa, Artificial Sequence
  RSV F Protein Variant
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNGQGRELPRFMNYTLNNAKKTNVTLSKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSNX

SEQ ID NO: 566 - DNA, 3450 bp, Artificial Sequence
  RSV F Protein Variant
atggagctgctgatcctgaaggccaacgccatcaccaccatcctgaccgcggtgaccttctgcttcgcctctggccagaacatcactgaggagttctacctacctcgacgactaggacttccggttgcggtagtggtggtaggactggcgccactggaagacgaagcggagaccggtcttgtagtgactcctcaagatggagagcacttgttccgctgtgagcaagggctacctgagcgccctgaggaccggttggtacaccagcgtgatcaccatcgagctgagcaacatcaagaagaatctcgtgaacaaggcgacactcgttcccgatggactcgcgggactcctggccaaccatgtggtcgcactagtggtagctcgactcgttgtagttcttcttcaagtgcaacggcaccgacgccaaggtgaagctgatcaagcaagagctggacaagtacaagaacgccgtgaccgagctgcaactgctgatgcagtcgactgttcacgttgccgtggctgcggttccacttcgactagttcgttctcgacctgttcatgttcttgcggcactggctcgacgttgacgactacgtcagctgacaagccaccaacaacggccagggcagagagctgccccgcttcatgaactacaccctgaacaacgccaagaagaccaacgtgaccctgtccaagaagaggagttcggtggttgttgccggtcccgtctctcgacggggcgaagtacttgatgtgggacttgttgcggttcttctggttgcactgggacaggttcttctcctagcgccgcttcctgggcttcctgctgggcgtgggctccgccattgccagtggcgtggccgtgtccaaggtgctgcacctggagggcgaggtgaacaagattcgcggcgaaggacccgaaggacgacccgcacccgaggcggtaacggtcaccgcaccggcacaggttccacgacgtggacctcccgctccacttgttctacaagagtgccctgctgtccactaacaaggccgtggtgagcctgagcaacggcgtgagtgtgctgactagcaaggtgctggacctgaagaactacatcgacgttctcacgggacgacaggtgattgttccggcaccactcggactcgttgccgcactcacacgactgatcgttccacgacctggacttcttgatgtagctgaagcaattgctgcccatcgtgaacaagcagtcctgtagcatctccaacatcgagactgtgatcgagttccagcagaagaacaaccgcctgctggaaatcattcgttaacgacgggtagcacttgttcgtcaggacatcgtagaggttgtagctctgacactagctcaaggtcgtcttcttgttggcggacgacctttagtcccgggagttcagtgtgaacgctggcgtgaccactcctgtctccacctacatgctgaccaacagcgagctgctgagcctgatcaacgacatgcccatcacgggccctcaagtcacacttgcgaccgcactggtgaggacagaggtggatgtacgactggttgtcgctcgacgactcggactagttgctgtacgggtagtgcaacgaccagaagaagcttatgtccaacaacgtgcagatcgtgaggcagcagagctactccatcatgagcatcatcaaggaggaggtgctggcctatgtggttgctggtcttcttcgaatacaggttgttgcacgtctagcactccgtcgtctcgatgaggtagtactcgtagtagttcctcctccacgaccggatacacgtgcagctgcccctgtacggcgtcatcgataccccttgctggaagctgcacaccagccccctgtgcaccaccaacaccaaggagggcagcaacatctgcccacgtcgacggggacatgccgcagtagctatggggaacgaccttcgacgtgtggtcgggggacacgtggtggttgtggttcctcccgtcgttgtagacggtgacccggaccgaccgcggctggtactgtgacaacgctggctcggtgagcttcttccctcaagctgaaacctgcaaggtccagagcaacagagtgttctgactgggcctggctggcgccgaccatgacactgttgcgaccgagccactcgaagaagggagttcgactttggacgttccaggtctcgttgtctcacaagactgacaccatgaactccctgaccctgccctccgaggtgaacctgtgcaacgtggatatcttcaaccccaagtatgactgcaagatcatgacctccaagaccactgtggtacttgagggactgggacgggaggctccacttggacacgttgcacctatagaagttggggttcatactgacgttctagtactggaggttctgggatgtctcgagctccgtgatcaccagcctgggcgccatcgtgtcctgctatggcaagaccaagtgcaccgccagcaacaagaaccggggcatcatcaagactacagagctcgaggcactagtggtcggacccgcggtagcacaggacgataccgttctggttcacgtggcggtcgttgttcttggccccgtagtagttctccttcagcaatgggtgcgactacgtttcgaacaagggcgtggacactgtgtccgtgggcaacaccctgtactacgtgaacaagcaagagggcaagagcctggaagtcgttacccacgctgatgcaaagcttgttcccgcacctgtgacacaggcacccgttgtgggacatgatgcacttgttcgttctcccgttctcggagtatgtgaagggcgagcccatcatcaacttctacgaccccctggtgttcccctccgacgaattcgacgcctccattagccaagtcaacgagaagatcaaccatacacttcccgctcgggtagtagttgaagatgctgggggaccacaaggggaggctgcttaagctgcggaggtaatcggttcagttgctcttctagttgcagagcctggccttcatccgcaagtccgacgagctgctgcacaacgtcaacgctggcaagagcaccaccaacatcatgatcaccaccatcatcatcgtgagtctcggaccggaagtaggcgttcaggctgctcgacgacgtgttgcagttgcgaccgttctcgtggtggttgtagtactagtggtggtagtagtagcacttcatcgtgatcctgctgagcctgatcgccgtgggcctgctgctgtactgcaaggcccggagcactcccgtgaccctgagcaaggaccagctgagcggcatagtagcactaggacgactcggactagcggcacccggacgacgacatgacgttccgggcctcgtgagggcactgggactcgttcctggtcgactcgccgtacaacaacatcgccttcagcaactgagttgttgtagcggaagtcgttgact

SEQ ID NO: 567 - protein, 550 aa, Artificial Sequence
  RSV F Protein Variant
MELLILKANAITTILTAVTFCFASGQNITEEFYQSTCSAVSKGYLSALRTGWYTSVITIELSNIKKNKCNGTDAKVKLIKQELDKYKNAVTELQLLMQSTQATNNKKRKRRFLGFLLGVGSAIASGVAVSKVLHLEGEVNKIKSALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNAGKSTTNIMITTIIIVIIVILLSLIAVGLLLYCKARSTPVTLSKDQLSGINNIAFSNX

SEQ ID NO: 568 - DNA, 3299 bp, Artificial Sequence
  RSV F Protein Variant
atggagctgctgatcctgaaggccaacgccatcaccaccatcctgaccgcggtgaccttctgcttcgcctctggccagaacatcactgaggagttctacctacctcgacgactaggacttccggttgcggtagtggtggtaggactggcgccactggaagacgaagcggagaccggtcttgtagtgactcctcaagatggagagcacttgttccgctgtgagcaagggctacctgagcgccctgaggaccggttggtacaccagcgtgatcaccatcgagctgagcaacatcaagaagaatctcgtgaacaaggcgacactcgttcccgatggactcgcgggactcctggccaaccatgtggtcgcactagtggtagctcgactcgttgtagttcttcttcaagtgcaacggcaccgacgccaaggtgaagctgatcaagcaagagctggacaagtacaagaacgccgtgaccgagctgcaactgctgatgcagtcgactgttcacgttgccgtggctgcggttccacttcgactagttcgttctcgacctgttcatgttcttgcggcactggctcgacgttgacgactacgtcagctgacaagccaccaacaacaagaagaggaagcgccgcttcctgggcttcctgctgggcgtgggctccgccattgccagtggcgtggccgtgtccaaggtgctgcgttcggtggttgttgttcttctccttcgcggcgaaggacccgaaggacgacccgcacccgaggcggtaacggtcaccgcaccggcacaggttccacgacgacctggagggcgaggtgaacaagatcaagagtgccctgctgtccactaacaaggccgtggtgagcctgagcaacggcgtgagtgtgctgactagcaaggttggacctcccgctccacttgttctagttctcacgggacgacaggtgattgttccggcaccactcggactcgttgccgcactcacacgactgatcgttccagctggacctgaagaactacatcgacaagcaattgctgcccatcgtgaacaagcagtcctgtagcatctccaacatcgagactgtgatcgagttccagcagcgacctggacttcttgatgtagctgttcgttaacgacgggtagcacttgttcgtcaggacatcgtagaggttgtagctctgacactagctcaaggtcgtcaagaacaaccgcctgctggaaatcacccgggagttcagtgtgaacgctggcgtgaccactcctgtctccacctacatgctgaccaacagcgagctgctgattcttgttggcggacgacctttagtgggccctcaagtcacacttgcgaccgcactggtgaggacagaggtggatgtacgactggttgtcgctcgacgactgcctgatcaacgacatgcccatcaccaacgaccagaagaagcttatgtccaacaacgtgcagatcgtgaggcagcagagctactccatcatgagcatcatcggactagttgctgtacgggtagtggttgctggtcttcttcgaatacaggttgttgcacgtctagcactccgtcgtctcgatgaggtagtactcgtagtacaaggaggaggtgctggcctatgtggtgcagctgcccctgtacggcgtcatcgataccccttgctggaagctgcacaccagccccctgtgcaccaccaacgttcctcctccacgaccggatacaccacgtcgacggggacatgccgcagtagctatggggaacgaccttcgacgtgtggtcgggggacacgtggtggttgaccaaggagggcagcaacatctgcctgacccggaccgaccgcggctggtactgtgacaacgctggctcggtgagcttcttccctcaagctgaaacctgcatggttcctcccgtcgttgtagacggactgggcctggctggcgccgaccatgacactgttgcgaccgagccactcgaagaagggagttcgactttggacgtaggtccagagcaacagagtgttctgtgacaccatgaactccctgaccctgccctccgaggtgaacctgtgcaacgtggatatcttcaaccccaagtatgatccaggtctcgttgtctcacaagacactgtggtacttgagggactgggacgggaggctccacttggacacgttgcacctatagaagttggggttcatactctgcaagatcatgacctccaagaccgatgtctcgagctccgtgatcaccagcctgggcgccatcgtgtcctgctatggcaagaccaagtgcaccgccagcgacgttctagtactggaggttctggctacagagctcgaggcactagtggtcggacccgcggtagcacaggacgataccgttctggttcacgtggcggtcgaacaagaaccggggcatcatcaagaccttcagcaatgggtgcgactacgtttcgaacaagggcgtggacactgtgtccgtgggcaacaccctgtactacgttgttcttggccccgtagtagttctggaagtcgttacccacgctgatgcaaagcttgttcccgcacctgtgacacaggcacccgttgtgggacatgatgctgaacaagcaagagggcaagagcctgtatgtgaagggcgagcccatcatcaacttctacgaccccctggtgttcccctccgacgaattcgacgcctccatacttgttcgttctcccgttctcggacatacacttcccgctcgggtagtagttgaagatgctgggggaccacaaggggaggctgcttaagctgcggaggtatagccaagtcaacgagaagatcaaccagagcctggccttcatccgcaagtccgacgagctgctgcacaacgtcaacgctggcaagagcaccaccaacatcatcggttcagttgctcttctagttggtctcggaccggaagtaggcgttcaggctgctcgacgacgtgttgcagttgcgaccgttctcgtggtggttgtagatgatcaccaccatcatcatcgtgatcatcgtgatcctgctgagcctgatcgccgtgggcctgctgctgtactgcaaggcccggagcactcccgtgaccctactagtggtggtagtagtagcactagtagcactaggacgactcggactagcggcacccggacgacgacatgacgttccgggcctcgtgagggcactgggtgagcaaggaccagctgagcggcatcaacaacatcgccttcagcaactgaactcgttcctggtcgactcgccgtagttgttgtagcggaatcgttgact

SEQ ID NO: 569 - protein, 25 aa, respiratory syncytial virus
RARRELPRFMNYTLNNAKKTNVTLS

SEQ ID NO: 570 - DNA, 6 bp, respiratory syncytial virus
aataaa

SEQ ID NO: 571 - DNA, 1725 bp, respiratory syncytial virus; identical to SEQ ID NO: 1

SEQ ID NO: 572 - DNA, 1726 bp, Artificial Sequence
  RSV F Protein Variant
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataaccatagaactaagtaatatcaagaaaaataagtgtaatggaacagatgccaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactggctcttatactgtaaggccagaagcacaccagtcacactaagcaaagatcaactgagtggtataaataatattgcatttagtaactaa

SEQ ID NO: 573 - DNA, 1722 bp, Consensus Sequence
atggagttgctaatcctcaaagcaaatgcaattaccacaatcctcactgcagtcacattttgttttgcttctggtcaaaacatcactgaagaattttatcaatcaacatgcagtgcagttagcaaaggctatcttagtgctctgagaactggttggtataccagtgttataacatagaataagtaatatcaagaaaaataagtgtaatggaacagatgcaaggtaaaattgataaaacaagaattagataaatataaaaatgctgtaacagaattgcagttgctcatgcaaagcacacaagcaacaaacaatcgagccagaagagaactaccaaggtttatgaattatacactcaacaatgccaaaaaaaccaatgtaacattaagcaagaaaaggaaaagaagatttcttggttttttgttaggtgttggatctgcaatcgccagtggcgttgctgtatctaaggtcctgcacctagaaggggaagtgaacaagatcaaaagtgctctactatccacaaacaaggctgtagtcagcttatcaaatggagttagtgtcttaaccagcaaagtgttagacctcaaaaactatatagataaacaattgttacctattgtgaacaagcaaagctgcagcatatcaaatatagaaactgtgatagagttccaacaaaagaacaacagactactagagattaccagggaatttagtgttaatgcaggtgtaactacacctgtaagcacttacatgttaactaatagtgaattattgtcattaatcaatgatatgcctataacaaatgatcagaaaaagttaatgtccaacaatgttcaaatagttagacagcaaagttactctatcatgtccataataaaagaggaagtcttagcatatgtagtacaattaccactatatggtgttatagatacaccctgttggaaactacacacatcccctctatgtacaaccaacacaaaagaagggtccaacatctgtttaacaagaactgacagaggatggtactgtgacaatgcaggatcagtatctttcttcccacaagctgaaacatgtaaagttcaatcaaatcgagtattttgtgacacaatgaacagtttaacattaccaagtgaagtaaatctctgcaatgttgacatattcaaccccaaatatgattgtaaaattatgacttcaaaaacagatgtaagcagctccgttatcacatctctaggagccattgtgtcatgctatggcaaaactaaatgtacagcatccaataaaaatcgtggaatcataaagacattttctaacgggtgcgattatgtatcaaataaaggggtggacactgtgtctgtaggtaacacattatattatgtaaataagcaagaaggtaaaagtctctatgtaaaaggtgaaccaataataaatttctatgacccattagtattcccctctgatgaatttgatgcatcaatatctcaagtcaacgagaagattaaccagagcctagcatttattcgtaaatccgatgaattattacataatgtaaatgctggtaaatccaccacaaatatcatgataactactataattatagtgattatagtaatattgttatcattaattgctgttggactgctcttatactgtaaggccagaagcacaccagtcacactaagcaaagatcaactgagtggtataaataatattgcatttagtaactaa

SEQ ID NO: 574 - DNA, 23 bp, Primer
ctgcagtcaccgtccttgacacc

(19) United States
(12) Patent Application Publication
(10) Pub. No.: US 2004/0161846 A1
(43) Pub. Date: Aug. 19, 2004

(54) Method of expression and agents identified thereby

(76) Inventors: Anthony John Mason, (US); Simon Peter Tucker, Victoria (AU); Paul R Young, Queensland (AU)

Correspondence Address: Karen B Dow, Morrison & Foerster, Suite 500, 3811 Valley Centre Drive, San Diego, CA, 92130-2332, US

(21) Appl. No.: 10/432,234

(22) PCT Filed: Nov. 22, 2001
(86) PCT No.: PCT/AU01/01517

(51) Int. Cl. C12N 15/86; C07K 14/005

(52) U.S. Cl. 435/456; 435/325; 435/69.1; 530/350

(57) ABSTRACT

The present invention relates generally to a method for the in vitro or in vivo production, by a eukaryotic host cell, of a protein from a negative sense single stranded RNA virus and, more particularly, to a method for the in vitro or in vivo production by a eukaryotic host cell of a protein from a virus of the family Paramyxoviradae and agents identified thereby. Still more particularly, said protein is the F, N, P or SH protein, the encoding nucleic acid molecule of which has been optimised for expression in a eukaryotic host cell. In yet another aspect, the present invention relates to a method for modulating the functional activity of an F protein. More particularly, said modulation is predicated on modulation of the functioning of a novel intrasequence cleavage event. In still another aspect, the protein expression product produced in accordance with the optimised expression method of the present invention and the method of modulating F protein functional activity are useful in a range of applications including, but not limited to, the identification, design and/or modification of agents capable of modulating functional activity of the subject protein. The proteins, encoding nucleic acid molecules and agents identified in accordance with the present invention are useful, inter alia, in the treatment and/or prophylaxis of viral infections.

### FIELD OF THE INENTION

The present invention relates generally to a method for the in vitro or in vivo production, by a eukaryotic host cell, of a protein from a negative sense single stranded RNA virus and, more particularly, to a method for the in vitro or in vivo production by a eukaryotic host cell of a protein from a virus of the family Paramyxoviradae and agents identified thereby. Still more particularly, said protein is the F, N, P or SH protein, the encoding nucleic acid molecule of which has been optimised for expression in a eukaryotic host cell. In yet another aspect, the present invention relates to a method for modulating the functional activity of an F protein. More particularly, said modulation is predicated on modulation of the functioning of a novel intrasequence cleavage event. In still another aspect, the protein expression product produced in accordance with the optimised expression method of the present invention and the method of modulating F protein functional activity are useful in a range of applications including, but not limited to, the identification, design and/or modification of agents capable of modulating functional activity of the subject protein. The proteins, encoding nucleic acid molecules and agents identified in accordance with the present invention are useful, inter alia, in the treatment and/or prophylaxis of viral infections.

### BACKGROUND OF THE INVENTION

Bibliographic details of the publications referred to by author in this specification are collected alphabetically at the end of the description.

The reference to any prior art in this specification is not, and should not be taken as, an acknowledgment or any form of suggestion that that prior art forms part of the common general knowledge in Australia.

Paramyxoviridae describes a family of enveloped viruses which exhibit a non-segmented, negative sense single stranded RNA genome. This family includes some significant pathogens of humans, animals and birds including the causel agents of measles, mumps, Newcastle disease, various respiratory diseases, Rinderpest and canine distemper.

Within this family exist two subfamilies (Paramyxovirinae and Pneumovirinae). Each subfamily comprises a number of genera—the genera of Pneumovirinae being Pneumovirus. In general, infection by these viruses occurs by fusion of the virus envelope with the plasma membrane of the host cell. Transcription and replication occur in the cytoplasm. Virions mature by budding through the host cell plasma membrane at sites containing the virus envelope proteins. Infected host cells commonly lyse, but temperate and persistent infections also occur. Infection of the host cell commonly results in cell fusion and syncytium formation, inclusions and haemadsorption.

The Pneumovirus genus of Paramyxoviridae differ from Rubulavirus, Morbillivirus and Paramyxovirus genera in that the members lack both haemagglutinin and neuraminidase activity. The Pneumovirus genus includes bovine and human respiratory syncytial virus amongst others. The latter virus is known to cause severe respiratory disease of humans whereas the former is an example of a family member responsible for animal diseases.

In general terms, the Paramyxovirus virion consists of a helical nucleocapsid, composed of genomic single stranded RNA and proteins NP, P and L, surrounded by an envelope containing a non-glycosylated M protein in the inner layer and two glycoproteins which extend across the width of the envelope and beyond the outer surface to form spikes. The larger of the envelope glycoproteins (often designated HN) exhibits cell binding, haemagglutinating and neuraminidase activities, while the smaller F (fusion) protein often exhibits haemolytic activity and promotes fusion between the virus envelope and the host plasma membrane. The F protein can also promote cell-cell fusion. The F protein is generally synthesised as an inactive precursor which is activated by proteolytic cleavage. In Pneumoviruses the G glycoprotein substitutes for HN.

Host cell infection is thought to occur by adsorption, via HN or G, to the cell surface, followed by F protein mediated fusion between the virus envelope and the host plasma membrane. Viral glycoproteins are also synthesised on membrane bound polysomes, glycosylated, and inserted into the host plasma membrane. During maturation, the virions bud through the region of the membrane containing these proteins. Accordingly, in terms of treating Paramyxoviridae virus infectivity, modulation of F protein functional activity provides a potential therapeutic mechanism since down-regulating or inhibiting F protein functioning would interfere with F protein mediated fusion of the virion with a potential host cell, and/or virion budding from cells which are already infected. However, in order to screen for agents which can modulate F protein functional activity, or to utilise F protein for any other purpose, it is necessary to establish an efficient and routinely reproducible in vitro system of producing recombinant F proteins, and in particular functionally active F proteins. To date this has proved elusive with existing expression systems producing only low levels of either inactive or very poorly active F proteins which often require co-expression with other viral glycoproteins to form syncytia. Further, to the extent that F protein is produced, albeit inactive or poorly active, only very low concentrations of protein products have been obtained.

The notion of codon usage is a poorly understood phenomenon which impacts on the efficiency of expression product production by given cells. Specifically, it has been determined that the levels of expression of protein produced by a cell can vary depending on the particular form of codon which is expressed in relation to a given amino acid. Although some amino acids are encoded by only one type of codon, other amino acids are encoded by up to six different codons, the efficiency of expression of which will vary depending on the host cell in which it is being expressed. It appears that certain types of cells exhibits preferences for expressing certain codon forms.

In work leading up to the present invention, the inventors have developed an in vitro expression system which both facilitates the production of functionally active F protein expression product and facilitate the production of significantly higher concentrations of F protein, or fragments thereof, than has been previously available. This system is based on identification by the inventors of two aspects of negative sense single stranded RNA viral protein expression which are compromised when the subject expression is performed in eukaryotic cells in vitro, these being inefficient codon usage and the presence of unwanted intrasequence mRNA splice sites.

With respect to the former aspect, the inventors have identified codons within the viral protein nucleic acid encoding molecule which are not efficiently expressed by a given eukaryotic host cell due to their not talking a form preferred by the host cell of interest. By establishing the form of codon preferably expressed by a given host cell, and modifying the viral protein encoding DNA sequence accordingly, the inventors have achieved levels of viral protein production, in particular F protein production, which have not, to date, been obtainable in normal mammalian expression systems. Further, the method of the present invention facilitates the production of functionally active F proteins.

In light of the fact that the basis and mechanism of codon usage preferences are not fully understood, there exist no conclusive theoretical principals by which one can predict with any certainty precisely which codons are not preferred by a given host cell nor which form they should ideally take. Accordingly, the successful development of viral protein encoding nucleic acid molecules which exhibits codons preferred by eukaryotic cells is a significant development.

With respect to the latter aspect of in vitro expression of the subject viral proteins, the inventors have further surprisingly determined that the in vitro expression of negative sense single stranded RNA viral proteins is compromised where in vitro expression is based on expression of a complementary DNA form of the naturally occurring RNA sequence encoding the protein of interest. This is due in part to the unexpected presence of RNA splice sites. Identification and removal of the unwanted splice sites has further facilitated efficient and increased viral protein production.

In a related aspect, and with respect to the F protein in particular, the inventors have identified a previously unknown intrasequence cleavage site which is involved in the -generation of functionally active F protein. Identification of this cleavage site now facilitates, inter alia, development of methods and identification of agents for modulation F protein cleavage and thereby methods of modulating F protein functioning.

The developments herein described now permit the identification and/or rational analysis, design and/or modification of agents for use in modulating viral protein functional activity and, in particular, F protein functional activity. Further, the developments of the present invention also facilitate generation of DNA and protein vaccines directed to the in vivo induction of an immune response to the subject proteins. The viral molecules produced in accordance with the method of the present invention and agents herein identified are useful inter alia, in a range of prophylactic and therapeutic applications relating to viral infections.

### SUMMARY OF THE INVENTION

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The subject specification contains nucleotide and amino acid sequence information prepared using the programme PatentIn Version 3.1, presented herein after the bibliography. Each nucleotide or amio acid sequence is identified in the sequence listing by the numeric indicator <210> followed by the sequence identifier (e.g. <210>1, <210>2, etc). The length, type of sequence (DNA, protein (PRT), etc) and source of organism for each nucleotide or amino acid sequence are indicated by information provided in the numeric indicator fields <211>, <212> and <213>, respectively. Nucleotide and amino acid sequences referred to in the specification are defined in the information provided in numeric indicator field <400> followed by the sequence identifier (e.g. <400>1, <400>2, etc). A summary of the sequence listings herein provided is detailed in Table 1.

Specific mutations in amino acid sequence are represented herein as “Xaa₁nXaa₂” where Xaa₁ is the original amino acid residue before mutation, n is the residue number and Xaa₂ is the mutant amino acid. The abbreviation “Xaa” may be the three letter or single letter amino acid code. A mutation in single letter code is represented, for example, by X₁nX₂ where X₁ and X₂ are the same as Xaa₁ and Xaa₂, respectively. The amino acid residues for F protein are numbered with the first residue R in the motif RARR being residue number 106.

One aspect of the present invention is directed to a method of facilitating production of a protein or derivative thereof from a negative sense single stranded RNA virus, said method comprising expressing in a host cell a nucleic acid molecule encoding said protein or derivative thereof, the nucleotide sequence of which nucleic acid molecule is optimised for expression by a eukaryotic cell.

Another aspect of the present invention provides a method of facilitating production of a protein or derivative thereof from a virus of the family Paramyxoviridae, said method comprising expressing in a host cell a nucleic acid molecule encoding said protein or derivative thereof the nucleotide sequence of which nucleic acid molecule is optimised for expression by a eukaryotic host cell.

Yet another aspect of the present invention provides a method of facilitating production of a protein or derivative thereof from a negative sense single stranded RNA virus, which protein directly or indirectly facilitates fusion of any one or more viral components with any one or more host cell components, said method comprising expressing in a host cell a nucleic acid molecule encoding said protein or derivative thereof, the nucleotide sequence of which nucleic acid molecule is optimised for expression by a eukaryotic cell.

Still another aspect of the present invention is therefore more particularly directed to a method of facilitating production of a F protein or derivative thereof from a negative sense single stranded RNA virus, said method comprising expressing in a host cell a nucleic acid molecule encoding said F protein or derivative thereof, the nucleotide sequence of which nucleic acid molecule is optimised for expression by a eukaryotic cell.

Yet still another aspect of the present invention provides a method of facilitating production of a N protein or derivative thereof from a negative sense single stranded RNA virus, said method comprising expressing in a host cell a nucleic acid molecule encoding said N protein or derivative thereof, the nucleotide sequence of which nucleic acid molecule is optimised for expression by a eukaryotic cell.

Still yet another aspect of the present invention provides a method of facilitating production of a P protein or derivative thereof from a negative sense single stranded RNA virus, said method comprising expressing in a host cell a nucleic acid molecule encoding said P protein or derivative thereof, the nucleotide sequence of which nucleic acid molecule is optimised for expression by a eukaryotic cell.

A further aspect provides a method of facilitating production of a SH protein or derivative thereof from a negative sense single stranded RNA virus, said method comprising expressing in a host cell a nucleic acid molecule encoding said SH protein or derivative thereof, the nucleotide sequence of which nucleic acid molecule is optimised for expression by a eukaryotic cell.

Another further aspect provides a method of facilitating production of a protein or derivative thereof from a negative sense single stranded RNA virus, said method comprising expressing in a mammalian host cell a nucleic acid molecule encoding said protein or derivative thereof, the nucleotide sequence of which nucleic acid molecule is optimised for expression by said mammalian host cell.

Yet another further aspect of the present invention is directed to a method of facilitating production of a protein or derivative thereof from a negative sense single stranded RNA virus, said method comprising expressing in a mammalian host cell a nucleic acid molecule encoding said protein or derivative thereof, the nucleotide sequence of which nucleic acid molecule is optimised for expression by said mammalian host cell wherein said optimisation is codon optimisation and/or nucleotide splice site deletion.

Still another further aspect provides a method of facilitating production of F protein or derivative thereof from respiratory syncytial virus, said method comprising expressing in a mammalian host cell a nucleic acid molecule encoding said F protein or derivative thereof, the nucleotide sequence of which nucleic acid molecule is optimised for expression by said mammalian host cell wherein said optimisation is nucleotide splice site deletion.

Still yet another further aspect of the present invention is directed to a method of facilitating production of a Fₛₒₗ portion of an F protein or derivative thereof from respiratory syncytial virus, said method comprising expressing in a host cell a nucleic acid molecule encoding said Fₛₒₗ portion or derivative thereof, the nucleotide sequence of which nucleic acid molecule is optimised for expression by said mammalian host cell wherein said optimisation is nucleotide splice site deletion.

Yet still another further aspect provides a method of facilitating production of F protein or derivative thereof from respiratory syncytial virus, said method comprising expressing in a mammalian host cell a nucleic acid molecule encoding said F protein or derivative thereof, the nucleotide sequence of which nucleic acid molecule is optimised for expression by said mammalian host cell wherein said optimisation is codon optimisation.

Another aspect of the present invention is directed to a method of facilitating production of a Fₛₒₗ portion of an F protein or derivative thereof from respiratory syncytial virus said method comprising expressing in a host cell a nucleic acid molecule encoding said Fₛₒₗ portion or derivative thereof, the nucleotide sequence of which nucleic acid molecule is optimised for expression by said mammalian host cell wherein said optimisation is codon optimisation.

Yet another aspect of the present invention provides a method of facilitating production of F protein or derivative thereof from respiratory syncytial virus, said method comprising expressing in a mammalian host cell a nucleic acid molecule encoding said F protein or derivative thereof, the nucleotide sequence of which nucleic acid molecule is optimised for expression by said mammalian host cell wherein said optimisation is nucleotide splice site deletion and codon optimisation.

Still another another aspect of the present invention provides a method of facilitating the production of a F protein or derivative thereof from a respiratory syncytial virus, said method comprising expressing in a host cell the nucleotide sequence set forth in<400>5 or derivative thereof.

Yet still another aspect provides a method of facilitating the production of a Fₛₒₗ portion of an F protein or derivative thereof from respiratory syncytial virus, said method comprising expressing in a host cell the nucleotide sequence set forth in <400>6 or derivative thereof.

Still yet another aspect provides a method of facilitating production of P protein or derivative thereof from respiratory syncytial virus, said method comprising expressing in a mammalian host cell a nucleic acid molecule encoding said P protein or derivative thereof, the nucleotide sequence of which nucleic acid molecule is optimised for expression by said mammalian host cell wherein said optimisation is codon optimisation.

A further aspect provides a method of facilitating the production of a P protein or derivative thereof from respiratory syncytial virus, said method comprising expressing in a host cell the nucleotide sequence set forth in<400>556 or derivative thereof.

Another further aspect provides a method of facilitating production of N protein or derivative thereof from respiratory syncytial virus, said method comprising expressing in a mammalian host cell a nucleic acid molecule encoding said N protein or derivative thereof, the nucleotide sequence of which nucleic acid molecule is optimised for expression by said mammalian host cell wherein said optimisation is codon optimisation.

Yet another further aspect provides a method of facilitating the production of a N protein or derivative thereof from respiratory syncytial virus, said method comprising expressing in a host cell the nucleotide sequence set forth in<400>559 or derivative thereof.

Still another further aspect provides a method of facilitating production of SH protein or derivative thereof from respiratory syncytial virus, said method comprising expressing in a mammalian host cell a nucleic acid molecule encoding said SH protein or derivative thereof, the nucleotide sequence of which nucleic acid molecule is optimised for expression by said mammalian host cell wherein said optimisation is codon optimisation.

Still yet another further aspect provides a method of facilitating the production of a SH protein or derivative thereof from respiratory syncytial virus, said method comprising expressing in a host cell the nucleotide sequence set forth in<400>562 or derivative thereof.

In another aspect, the present invention should be understood to extend to the optimised nucleic acid molecules described herein and to the expression products derived therefrom.

Yet another aspect of the present invention is directed to a method of regulating the functional activity of a viral F protein, which protein in its non-fully functional form comprises an F2 portion linked, bound or otherwise associated with an F1 portion, which F2 portion comprises an intervening peptide sequence, said method comprising modulating cleavage of said intervening peptide sequence wherein excision of at least part of said intervening sequence from said non-fully functional form of said F protein up-regulates F protein functional activity.

Still another aspect of the present invention is directed to a method of regulating the functional activity of a Paramyxoviridae derived F protein, which protein in its non-fully functional form comprises an F2 portion linked, bound or otherwise associated with an F1 portion, which F2 portion comprises an intervening peptide sequence, said method comprising modulating cleavage of said intervening peptide sequence wherein excision of at least part of said intervening sequence from said non-fully functional form of said F protein up-regulates F protein functional activity.

Yet still another aspect of the present invention provides a method of regulating the functional activity of a respiratory syncytial virus F protein, which protein in its non-fully functional form comprises an F2 portion linked, bound or otherwise associated with an F1 portion, which F2 portion comprises an intervening peptide sequence, said method comprising modulating cleavage of said intervening peptide sequence, wherein excision of at least part of said intervening sequence from said non-fully functional form of said F protein up-regulates F protein functional activity and wherein said cleavage events occur at the cleavage sites defined by the peptide sequences RARR (<400>564) and KKRKRR (<400>563).

In a related aspect, the present invention provides a method of regulating the functional activity of a viral F protein, which protein in its non-fully functional form comprises the structure: X₁, X₂, X₃

wherein:

X₁ comprises the non-intervening peptide sequence region of the F2 portion;

X₂ comprises the intervening peptide sequence region of the F₂ portion; and

X₃ comprises the F1 portion

said method comprising modulating cleavage of said intervening peptide sequence wherein excision of at least part of said intervening sequence from said non-fully functional form of said F protein up-regulates F protein functional activity.

Still yet another aspect provides a method of inhibiting, retarding or otherwise down-regulating the functional activity of a Paramyxoviridae derived F protein, which protein in its non-fully functional form comprises an F2 portion linked, bound or otherwise associated with an F1 portion, which F2 portion comprises an intervening peptide sequence, said method comprising inhibiting or otherwise down-regulating cleavage of said intervening peptide sequence.

A further aspect of the present invention provides a method of down-regulating the functional activity of a Paramyxoviradae derived F protein, which protein in its non-fully functional form comprises the structure:

X₁X₂X₃

wherein:

X₁ comprises the non-intervening peptide sequence region of the F2 portion;

X₂ comprises the intervening peptide sequence region of the F2 portion; and

X₃ comprises the F1 portion

said method comprising inhibiting or otherwise down-regulating cleavage of said intervening peptide sequence.

Another further aspect provides a method for detecting an agent capable of regulating the functional activity of a viral F protein or derivative thereof said method comprising contacting a eukaryotic cell expressing an optimised nucleic acid molecule encoding said viral F protein or derivative thereof, as hereinbefore described, with a putative modulatory agent and detecting an altered expression phenotype and/or functional activity.

In yet another aspect there is provided a method for detecting an agent capable of regulating the functional activity of a viral F protein or derivative thereof said method comprising contacting a host cell, which host cell expresses a nucleic acid molecule encoding the non-fully functional form of said viral F protein or derivate thereof as hereinbefore described, with a putative modulatory agent and detecting an altered expression phenotype and/or altered functional activity wherein said agent modulates cleavage of the intervening peptide sequence.

Still another further aspect of the present invention is directed to a method for analysing, designing and/or modifying an agent capable of interacting with a viral F protein or derivative thereof and modulating at least one functional activity associated with said protein, which protein is produced in accordance with the method of the present invention said method comprising contacting said F protein or derivate thereof with a putative agent and assessing the degree of interactive complementarity of said agent with said protein.

Still yet another further aspect of the present invention is directed to an agent capable of interacting with a viral F protein and modulating at least one functional activity associated with said viral protein.

In still another aspect there is provided a viral F protein variant comprising a mutation in the intervening peptide sequence wherein said variant exhibits modulated functional activity relative to wild type F protein or a derivative, homologue, analogue, chemical equivalent or mimetic of said variant.

Another aspect of the present invention provides a viral F protein variant comprising a mutation in the intervening peptide sequence wherein said variant exhibits down-regulated functional activity relative to wild type F protein or a derivative, homologue, analogue, chemical equivalent or mimetic of said variant.

Yet another aspect provides a respiratory syncytial virus F protein variant comprising a mutation in the cleavage site defined by amino acids RARR (<400>564) wherein said variant exhibits down-regulated functional activity relative to wild type F protein or a derivative, homologue, analogue, chemical equivalent or mimetic of said variant.

Preferably said mutation comprises one or more of the amino acid substitutions selected from the following list:

(i) R106G

(ii) A107Q

(iii) R108G

Still more preferably said F protein variant comprises the sequence substantially as set forth in<400>565.

Still another aspect provides a respiratory syncytial virus F protein variant comprising a multiple amino acid deletion from the intervening peptide sequence wherein said variant exhibits down-regulated functional activity relative to wild type F protein or a derivative, homologue, analogue, chemical equivalent of said variant.

It is more preferably provided that said amino acid deletion is a partial deletion of the intervening peptide sequence and more preferably a deletion of the peptide sequence

RARRELPRFMNYTLNNAKKTNVTLS <400>569.

Still more preferably said variant comprises the amino acid sequence substantially as set forth in <400>567.

Yet still another aspect of the present invention is directed to an isolated nucleic acid molecule selected from the list consisting of:

(i) An isolated nucleic acid molecule or derivative or equivalent thereof comprising a nucleotide sequence encoding or complementary to a sequence encoding a viral F protein variant or derivative, homologue, analogue, chemical equivalent or mimetic of said variant, which variant comprises a mutation in the intervening peptide sequence wherein said variant exhibits modulated functional activity relative to wild-type F protein.

(ii) An isolated nucleic acid molecule or derivative or equivalent thereof comprising a nucleotide sequence encoding or complementary to a sequence encoding a viral F protein variant or derivative, homologue, analogue, chemical equivalent or mimetic of said variant, which variant comprises a mutation in the intervening peptide sequence wherein said variant exhibits down-regulated functional activity relative to wild-type F protein.

(iii) An isolated nucleic acid molecule or derivative or equivalent thereof comprising a nucleotide sequence encoding or complementary to a sequence encoding a respiratory syncytial virus F protein or derivative, homologue, analogue, chemical equivalent or mimetic of said variant, which variant comprises a mutation in the cleavage site defined by amino acids. RARR wherein said variant exhibits down-regulated functional activity relative to wild-type F protein.

(iv) An isolated nucleic acid molecule or derivative or equivalent thereof comprising a nucleotide sequence encoding or complementary to a sequence encoding a respiratory syncytial virus F protein variant or derivative, homologue, analogue, chemical equivalent or mimetic of said variant, which variant comprises one or more of the amino acid substitutions selected from the following list:

(a) R106G

(b) A107Q

(c) R108G

(v) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises a multiple amino acid deletion from the intervening peptide sequence wherein said variant exhibits down-regulated functional activity relative to wild-type F protein.

(vi) An isolated nucleic acid molecule or derivative or analogue thereof comprising a nucleotide sequence encoding or complementary to a sequence encoding a viral F protein valiant or derivative, homologue, analogue, chemical equivalent or mimetic of said variant, which variant comprises a partial deletion of the intervening peptide sequence and more preferably a deletion of the peptide sequence

RARRELPRFMNYTLNNAKKTNVTLS <400>569.

(vii) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises the amino acid sequence substantially as set forth in<400>567.

(viii) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises the amino acid sequence substantially as set forth in<400>565.

(ix) An isolated nucleic acid molecule or derivative or analogue thereof comprising the nucleotide substantially as set forth in<400>568.

(x) An isolated nucleic acid molecule or derivative or analogue thereof comprising the nucleotide substantially as set forth in<400>566.

Still yet another aspect of the present invention provides a recombinant viral construct comprising a nucleic acid molecule encoding a viral F protein or derivative thereof, the nucleotide sequence of which nucleic acid molecule comprises codons optimised for expression in a eukaryotic cell, wherein said recombinant viral construct is effective in inducing, enhancing or otherwise stimulating an immune response to said F protein.

A further aspect of the present invention provides a recombinant viral construct comprising a nucleic acid molecule encoding a viral F protein variant or derivative thereof wherein said recombinant viral construct is effective in inducing, enhancing or otherwise stimulating an immune response to said F protein variant.

Another further aspect of the present invention relates to a vaccine comprising a recombinant viral construct which construct comprises a nucleic acid molecule encoding a respiratory syncytial virus F protein or derivative thereof, the nucleotide sequence of which nucleic acid molecule is optimised for expression in a eukaryotic cell wherein said recombinant viral construct is effective in inducing, enhancing or otherwise stimulating an immune response to said F protein.

Yet another further aspect of the present invention relates to a vaccine comprising a recombinant viral construct which construct comprises a nucleic acid molecule encoding a respiratory syncytial virus F protein variant or derivative thereof, wherein said recombinant viral construct is effective in inducing, enhancing or otherwise stimulating an immune response to said F protein variant.

In accordance with these aspects of the present invention, the nucleotide sequence of the subject nucleic acid molecule is preferably the nucleotide sequence defined in <400>5, <400>6, <400>566 or <400>568.

Still another further aspect of the present invention provides the method of modulating at least one functional activity associated with a viral F protein in a subject, said method comprising introducing into said subject and effective amount of an F protein modulatory agent for a time and under condition sufficient for said agent to interact with said F protein.

Still yet another further aspect of the present invention provides a method of modulating at least one functional activity associated with a viral F protein, said method comprising contacting said viral F protein with an effective amount of an F protein modulatory agent for a time and under conditions sufficient for said agent to interact with said F protein.

Yet still another further aspect of the present invention relates to a method for the treatment and/or prophylaxis of a condition characterised by infection with a negative sense single stranded RNA virus in a subject, said method comprising administering to said subject an effective amount of an agent, which agent is capable of down-regulating at least one functional activity of the F protein expressed by said virus, for a time and under conditions sufficient for said agent to interact with said F protein.

In still yet another aspect, the present invention relates to a method for the treatment and/or prophylaxis of a condition characterised by infection with a negative sense single stranded RNA virus in a subject, said method comprising administering to said subject an effective amount of a composition comprising an F protein or derivative thereof, F protein variant or derivative thereof and/or a nucleic acid molecule encoding said F protein or F protein variant as hereinbefore defined or a derivative, homologue, analogue, chemical equivalent a mimetic of said protein or nucleic acid molecule for a time and under conditions sufficient for said composition to down-regulate said viral F protein functional activity.

In another aspect the present invention relates to the use of an agent capable of modulating at least one functional activity of a viral F protein, which agent is identified and/or generated in accordance with the methods hereinbefore defined, in the manufacture of a medicament for the treatment and/or prophylaxis of a condition characterised by infection with a negative sense single stranded RNA virus.

In still another aspect the present invention relates to the use of a composition comprising an F protein or derivative thereof, F protein variant or derivative thereof, nucleic acid molecule encoding said F protein or F protein variant as hereinbefore defined or a derivative, homologue, analogue, chemical equivalent or mimetic of said protein or nucleic acid molecule, in the manufacture of a medicament for the treatment and/or prophylaxis of a condition characterised by infection with a negative sense single stranded RNA virus.

In another aspect the present invention relates to the use of an agent, which agent is identified in accordance with the methods hereinbefore defined, in the manufacture of a medicament for the modulation of at least one viral F protein associated functional activity.

Yet another aspect relates to agents for use in modulating the functional activity of a viral F protein wherein said agent is identified in accordance with the methods hereinbefore defined.

Still yet another aspect relates to agents for use in the treatment and/or prophylaxis of a condition characterised by infection with a negative sense single stranded RNA virus wherein said agent is identified in accordance with the methods hereinbefore defined.

Yet still another aspect relates to a composition comprising an F protein or derivative thereof, F protein variant or derivative thereof, a nucleic acid molecule encoding said F protein or F protein variant as hereinbefore defined or a derivative, homologue, analogue, chemical equivalent or mimetic of said protein or nucleic acid molecule for use in the treatment and/or prophylaxis of a condition characterised by infection with a negative sense single stranded RNA virus.

In yet another aspect the present invention relates to a pharmaceutical composition comprising an active ingredient, as hereinbefore defined, and one or more pharmaceutically acceptable carriers and/or diluents.

Single and three letter abbreviations used throughout the specification are defined in Table 2.

TABLE 2
Single and three letter amino acid abbreviations

| | Three-letter | One-letter |
|---|---|---|
| Amino Acid | Abbreviation | Symbol |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | The | T |
| Tryptophan | Trp | W |
| Tyrosine | Tyr | Y |
| Valine | Val | V |
| Any residue | Xaa | X |

### BRIEF DESCRIPTION OF THE DRAWINGS

FIG. 1a is a schematic representation of the 574 amino acid sequence of the human RSV fusion protein F. Amino acid numbers 1-22 comprises the signal sequence. The F2 subunit comprises amino acid numbers 23-130. The fusion cleavage (site 1) is amino acid numbers 131-136. Site 2 comprises residues 106-109. The F1 subunit comprises residues 136-574. The transmembrane domain is believed to span residues 525-548. The cytoplasmic domain comprises residues 549-574.

FIG. 1b is a schematic representation of the amino acid sequence of the 524 residue soluble F protein. This protein is referred to as Fₛₒₗ. Fₛₒₗ is formed by expressing the coding sequence for F minus the residues encoding the transmembrane domain and the cytoplasmic domain of F.

FIG. 1c is a schematic representation of F and Fₛₒₗ. Cleavage positions of site 1 and site 2 are designated. Hydrophobic regions are shaded in black (from left to right, signal sequence, fusion peptide and transmembrane domain). Downward facing flags designate positions of potential N-linked glycosylation sites. The 24 amino acid region bounded by cleavage sites 1 and 2 is shown as a cross-hatched region.

FIG. 2a is a schematic representation of the alignment of sequences coding for the human RSV F protein. F.viral refers to the sequence as found in wild type A2 RSV strain. F refers to the sequence which differs in 27/1725 positions from the viral sequence. Those changes where made in order to introduce unique restriction sites to the sequence. F.opt. refers to the F coding sequence which has been changed to allow for higher expression levels as outlined in the accompanying application. A total of 378/1725 nucleotides have been changed from the F.viral sequence. Underneath the boxed sequence a consensus sequence is shown.

FIG. 2b is a schematic representation of the alignment of sequences coding for the human RSV Fₛₒₗ protein. F.ₛₒₗ.viral refers to the sequence as found in the wild type A2 RSV strain. F.ₛₒₗ refers to the sequence which differs from the viral sequence in 24/1575 nucleotides. All of these changes were incorporated to introduce unique restriction sites. F.ₛₒₗ.opt. refers to the Fₛₒₗ coding sequence optimised as described herein. A total of 334/1575 nucleotides have been changed. A consensus is shown under the boxed sequences.

FIGS. 3a and b are schematic representations of the DNA sequences optimised for expression as cloned in the expression vector pCICO.F.FL.opt (a) and pCICO.F.opt (b). The plasmid pCICO.F.FL.opt contains the sequence referred to in FIG. 2a as F.opt. The plasmid pCICO.F.opt contains the sequence referred to in FIG. 2b as F.ₛₒₗ.opt. 5′ and 3′ untranslated sequences not included in the FIG. 2 sequences are shown in this Figure.

FIGS. 4a and b are schematic representations of the construction of F and Fₛₒₗ expression vectors. These diagrams describe in detail the steps involved in constructing expression vectors pCICO.F.FL.opt and pCICO.F.opt. See text of examples for details. As previously noted pCICO.F.FL.opt contains the optimised sequence F.opt. (FIG. 2a) and pCICO.F.opt contains the optimised sequence F.ₛₒₗ.opt (FIG. 2b).

FIG. 5 is an image of an autoradiograph of a 10% SDS-PAGE gel of a immunoprecipitation of 35-5 labelled supernatents from 293 cells transfected with lane (a) pCICO.FS3 (containing viral Fₛₒₗ sequence) lane (b) pCICO.F.opt (containing optimised Fₛₒₗ sequence). Lane (c) is from mock-tranfected cells. Lane (d) contains readioactively labelled molecular weight markers. The Fₛₒₗ protein migrates at approximately 60 kd in size.

FIG. 6 is a schematic representation of the alignment of sequences coding for the human RSV F protein. F.viral refers to the sequence as found in wild type A2 RS strain (<400>571). F.nat refers to the sequence found in a RSV A2 cDNA clone assembled in these studies (<400>572). The two sequences differ in two places (nt 174 and 222) which does not effect the coding potential. Underneath the boxed sequence a consensus sequence is shown (<400>573).

FIG. 7 is a western blot of protein samples derived from 293 cells transfected with WT (pCICO.F.FL.opt), A2 (pCICO.F.nat) and Ctrl (control) plasmids. Cells were havested at 24, 48 and 72 hours post transfection. Cell lysates were analysed by 12% polyacrylamide SDS-PAGE and after electrophoresis proteins were electroblotted onto a nitrocellulose membrane. F protein was detected as described in example 5. The immuno-reactive F bands F1 and F1′ are indicated by arrows. The position of molecular weight markers is shown.

FIG. 8 is photographs of 293 cells transfected with pCICO.F.FL.opt (a), pCICO.F.nat (b) and control plasmid (c). Photographs were taken 48 hours post transfection and the magnification is 400×. Figures a, b and c flow from top to bottom.

### DETAILED DESCRIPTION OF THE INVENTION

The present invention is predicated, in part, on the development of a negative sense single stranded RNA viral protein expression system based on optimisation of expression of the viral protein encoding nucleic acid sequence such that expression of the subject nucleic acid molecule sequence by a given eukaryotic host cell is facilitated and/or improved. In a related aspect, the inventors have identified a novel cleavage site in the F viral protein, the cleavage of which is thought to be essential for the generation of a fully functionally active F protein. These developments now permit the recombinant production of viral proteins and the identification and design of agents for use in modulating functional activity of the subject proteins.

Accordingly, one aspect of the present invention is directed to a method of facilitating production of a protein or derivative thereof from a negative sense single stranded RNA virus, said method comprising expressing in a host cell a nucleic acid molecule encoding said protein or derivative thereof, the nucleotide sequence of which nucleic acid molecule is optimised for expression by a eukaryotic cell.

Reference to a “negative sense single stranded RNA virus” should be understood as a reference to any negative sense single stranded RNA virus, and includes, but is not limited to, viruses of the family Paramyxoviridae, Rhabdoviridae, Filoviridae, Orthomyxoviridae, Bunyaviridae or Arenaviridae. Preferably, said negative sense single stranded RNA virus is of the family Paramyxoviridae. Without limiting the present invention to any one theory or mode of action, viruses of the family Paramyxoviridae are cytoplasm replicating viruses. In this regard, RNA replication involves mRNA transcription from the genomic RNA via the virion transcriptase. Utilising the protein products of this transcription, there follows the production of a full length positive stranded template which is used for the synthesis of genomic RNA. The genome is transcribed from the the 3′ end by virion associated enzymes into mRNAs. Replication takes place in the cytoplasm and assembly occurs via budding on the plasma membrane. The subject budding occurs through the host cell plasma membrane at sites containing the virus envelope proteins.

Accordingly, there is more particularly provided a method of facilitating production of a protein or derivative thereof from a virus of the family Paramyxoviridae, said method comprising expressing in a host cell a nucleic acid molecule encoding said protein or derivative thereof, the nucleotide sequence of which nucleic acid molecule is optimised for expression by a eukaryotic host cell.

Still more preferably, said virus is of the sub-family Pneumovirinae and most preferably said virus is respiratory syncytial virus.

Reference to a “protein from a negative sense single stranded RNA virus” should be understood as a reference to any protein which is expressed by the subject virus or a derivative of said protein. Examples of proteins include, but are not limited to, nucleocapsid associated proteins such as RNA binding proteins (e.g. N, NP), phosphoproteins (e.g. P), polymerase proteins (e.g. L), or envelope proteins (e.g. F, G, H, HN or SH). It should be understood that the subject protein may exist, in its naturally occurring form, either in isolation or fused or otherwise linked to any other proteinaceous or non-proteinaceous molecule. Preferably, the subject protein is a fusion protein, N, P or SH.

Accordingly, in one embodiment there is provided a method of facilitating production of a protein or derivative thereof from a negative sense single stranded RNA virus, which protein directly or indirectly facilitates fusion of any one or more viral components with any one or more host cell components, said method comprising expressing in a host cell a nucleic acid molecule encoding said protein or derivative thereof, the nucleotide sequence of which nucleic acid molecule is optimised for expression by a eukaryotic cell.

Reference to a viral protein which “directly or indirectly facilitates fusion of any one or more viral components with any one or more host cell components” should be understood as a reference to any viral protein which functions to induce or otherwise contribute to the fusion of one or more viral molecules (such as a protein or structural component) with any one or more host cell molecules. It should be understood that this activity may comprise any one of a number of functional activities attributable to the subject protein, which other activities are not necessarily related to fusion. It should also be understood that the subject functional activity may either directly facilitate fusion or it may induce or otherwise contribute to the functioning of an unrelated molecule, which unrelated molecule directly facilitates the subject fusion. Preferably the viral protein is an F protein.

This embodiment of the present invention is therefore more particularly directed to a method of facilitating production of a F protein or derivative thereof from a negative sense single stranded RNA virus, said method comprising expressing in a host cell a nucleic acid molecule encoding said F protein or derivative thereof, the nucleotide sequence of which nucleic acid molecule is optimised for expression by a eukaryotic cell.

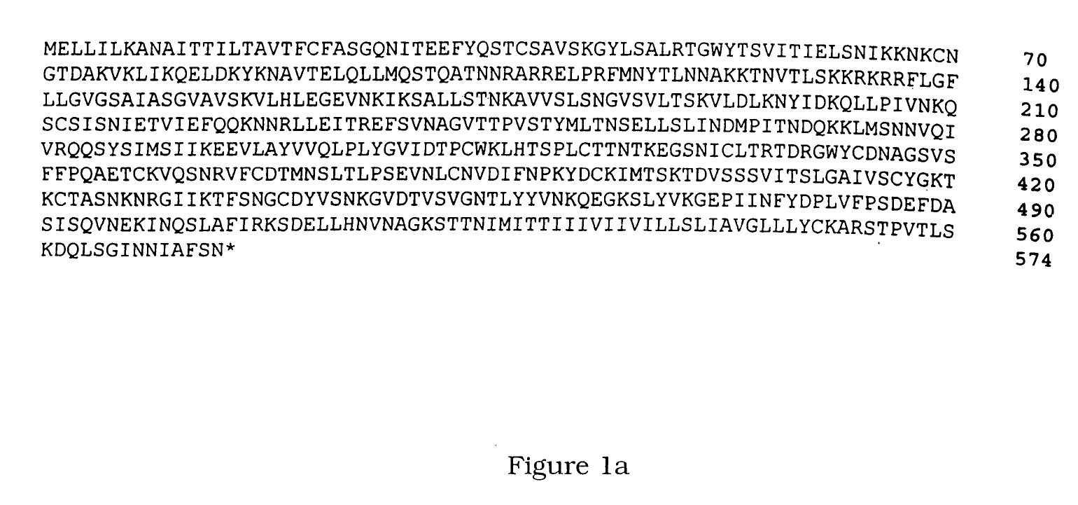

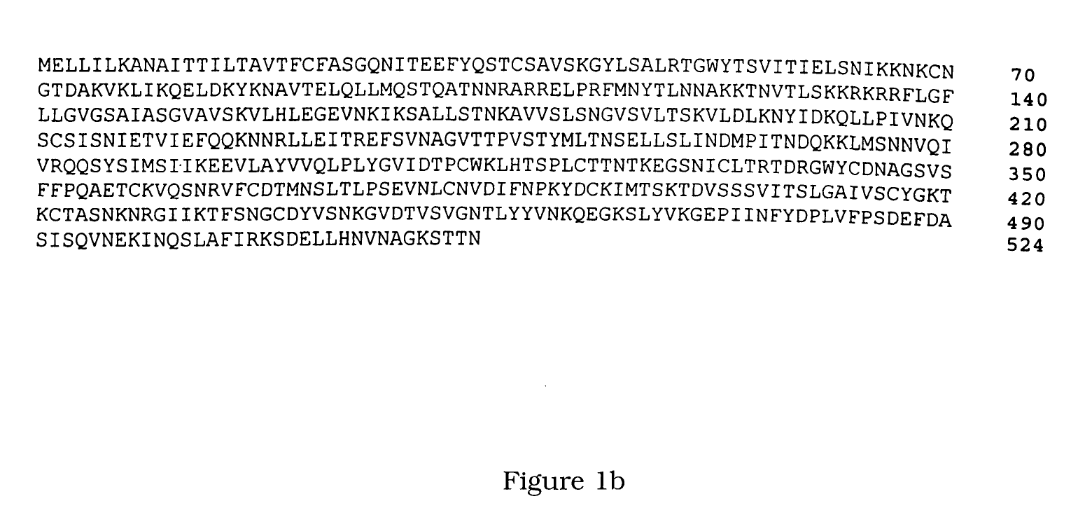

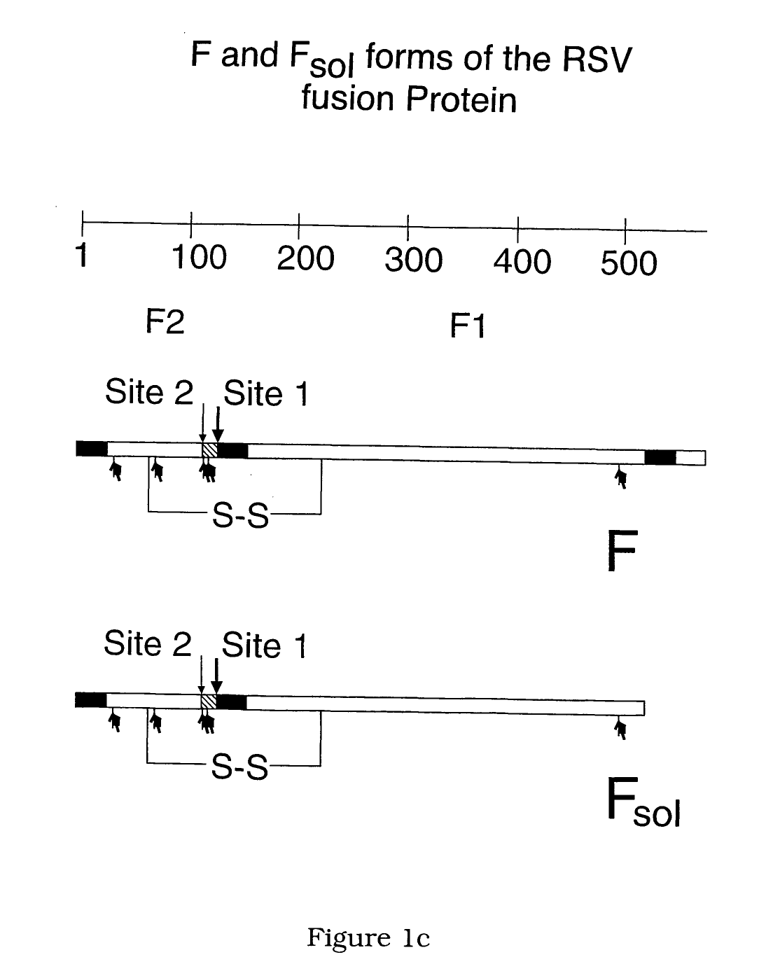

Reference to a “F protein” should be understood as a reference to the viral molecule which, inter alia, facilitates fusion between the virus envelope and the host cell plasma membrane of infected cells. The term “F protein” should be understood to encompass all forms of F protein including, for example, any mutant, polymorphic or homologous forms of F protein. Without limiting the present invention in any way, the F protein generally comprises, at the amino terminus, an F2 portion which is linked to an F1 portion. The F1 contains a transmembrane region of the molecule which is, in turn, linked to an extracellular portion of the F protein. The cytoplasmic portion of the F protein comprises the carboxy terminus. As detailed earlier, the F protein is generally synthesised in a precursor form which is activated by proteolytic cleavage at the F2/F1 junction. It is though that this cleavage step reveals a fusion peptide which interacts with the host cell. The F2/F1 junction of the respiratory syncytial virus F protein is shown in FIG. 1.

In another embodiment there is provided a method of facilitating production of a N protein or derivative thereof from a negative sense single stranded RNA virus, said method comprising expressing in a host cell a nucleic acid molecule encoding said N protein or derivative thereof, the nucleotide sequence of which nucleic acid molecule is optimised for expression by a eukaryotic cell.

In yet another preferred embodiment there is provided a method of facilitating production of a P protein or derivative thereof from a negative sense single stranded RNA virus, said method comprising expressing in a host cell a nucleic acid molecule encoding said P protein or derivative thereof, the nucleotide sequence of which nucleic acid molecule is optimised for expression by a eukaryotic cell.

In still yet another preferred embodiment there is provided a method of facilitating production of a SH protein or derivative thereof from a negative sense single stranded RNA virus, said method comprising expressing in a host cell a nucleic acid molecule encoding said SH protein or derivative thereof, the nucleotide sequence of which nucleic acid molecule is optimised for expression by a eukaryotic cell.

Preferably, the negative sense single stranded RNA virus of these preferred embodiments of the present invention is a virus of the family Paramyxoviridae. More preferably the virus is of the sub-family Pneumovirinae and still more preferably the subject virus is a virus of the genus Pneumovirus. Most preferably, the virus is respiratory syncytial virus.

To the extent that it is not otherwise specified, reference to a viral “protein” extends to derivatives thereof.

“Derivatives” of the subject protein include fragments, parts, portions, mutants, variants and mimetics thereof including fusion proteins. Parts or fragments include, for example, active regions of the subject protein. In one aspect of the present invention, for example, the subject protein is a F protein which does not comprise the transmembrane and cytoplasmic portions (herein referred to as Fₛₒₗ). The Fₛₒₗ fragment of the F protein is useful for X-ray crystallography and other forms of modelling for purposes such as rational drug design. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in the sequence has been removed and a different residue inserted in its place. An example of substitutional amino acid variants are conservative amino acid substitutions. Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

The derivatives include fragments having particular portions of the entire protein fused to peptides, polypeptides or other proteinaceous or non-proteinaceous molecules.

“Mutants” include variants of the subject protein which variants exhibit modified sequences, structures and/or functions. For example, the F protein variants described herein, which variants exhibit amino acid sequence alterations leading to altered cleavage properties, fall within the scope of the term “mutants”.

The term “protein” should be understood to encompass peptides, polypeptides and proteins. The protein may be glycosylated or unglycosylated and/or may contain a range of other molecules fused, linked, bound or otherwise associated to the protein such as amino acids, lipids, carbohydrates or other peptides, polypeptides or proteins. Reference hereinafter to a “protein” includes a protein comprising a sequence of amino acids as well as a protein associated with other molecules such as amino acids, lipids, carbohydrates or other peptides, polypeptides or proteins.

The method of the present invention is predicated on the production of a viral protein by expressing a nucleic acid molecule as herein described. In this regard, the term “expressing” should be understood to refer to the transcription and translation of a nucleic acid molecule resulting in the synthesis of a peptide, polypeptide or protein expression product. The synthesis of an expression product via the translation step of nucleic acid molecule expression is herein referred to as “production” of that expression product.

The viral protein encoding nucleic acid molecule of the present invention is expressed in a eukaryotic host cell. By “host cell” is meant any eukaryotic cell which can be transformed or transfected with a nucleotide sequence. Preferred eukaryotic host cells are mammalian cells and even more preferably 293 cells and Chinese Hamster Ovary cells.

Accordingly, there is provided a method of facilitating production of a protein or derivative thereof from a negative sense single stranded RNA virus, said method comprising expressing in a mammalian host cell a nucleic acid molecule encoding said protein or derivative thereof, the nucleotide sequence of which nucleic acid molecule is optimised for expression by said mammalian host cell.

Preferably, the subject protein is a fusion protein (more particularly the F protein), N, P or SH.

Preferably, the negative sense single stranded RNA virus of these preferred embodiments of the present invention is a virus of the family Paramyxoviridae. More preferably the virus is of the sub-family Pneumovirinae and still more preferably the subject virus is a virus of the genus Pneumovirus. Most preferably, the virus is respiratory syncytial virus.

The nucleic acid molecule which is expressed in accordance with the method of the present invention encodes a viral protein or derivative thereof. By “encodes” is meant that the expression product comprises the subject protein or derivative. However, it should be understood that this is not intended as a restriction in any way on the diversity of the subject expression product other than that it should comprise the subject protein or derivative thereof. For example, the nucleic acid molecule which is introduced into the host cell may encode the protein fused to another protein, peptide or polypeptide (which is consistent with the definition of protein “derivative” as hereinbefore provided) or the nucleic acid molecule may encode multiple proteins wherein at least one of those proteins is the subject protein or derivative thereof.

Reference to the subject nucleic acid molecule being “optimised” for expression by a eukaryotic host cell should be understood as a reference to a nucleic acid molecule which has been mutated or otherwise varied such that its recombinant expression by a eukaryotic host cell is facilitated. Said “facilitation” includes, but is not limited to, inducing or improving levels of protein expression and/or functional activity of the expression product. Preferably, said optimisation takes the form of codon optimisation and/or nucleotide splice site deletion.

By “codon optimisation” is meant that at least one codon of the naturally occurring viral protein encoding nucleotide sequence has been altered such that it encodes the same amino acid as the naturally occurring codon but uses an alternative codon to that which naturally encodes the subject amino acid, which alternative codon form is more preferably expressed by a eukaryotic cell than the naturally occurring codon form.

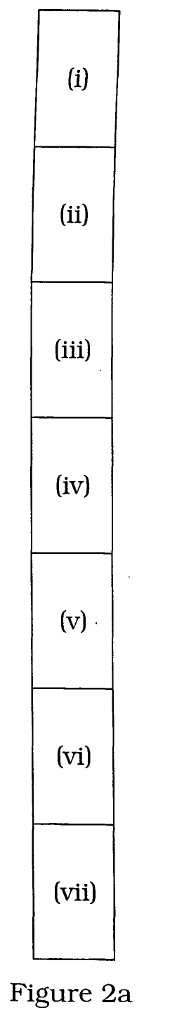

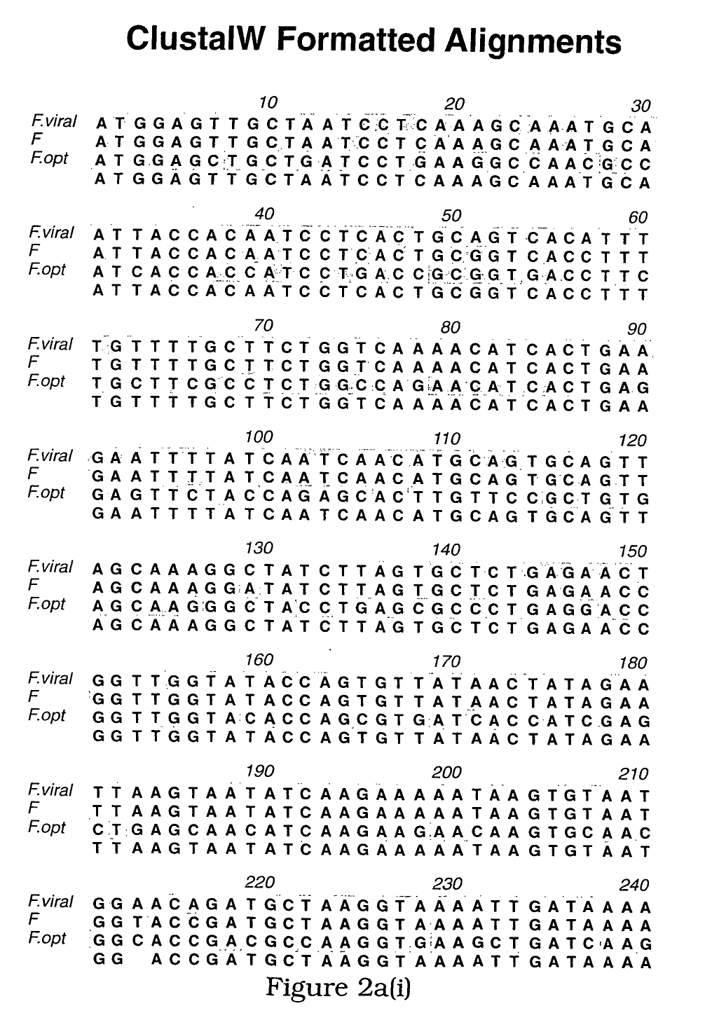

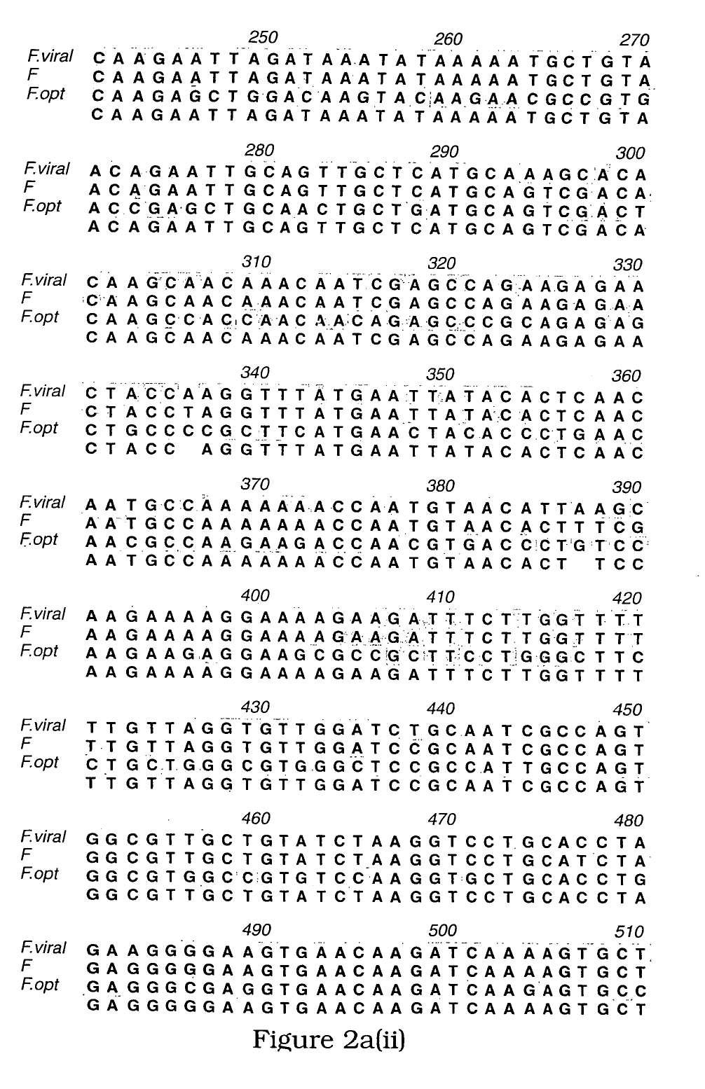

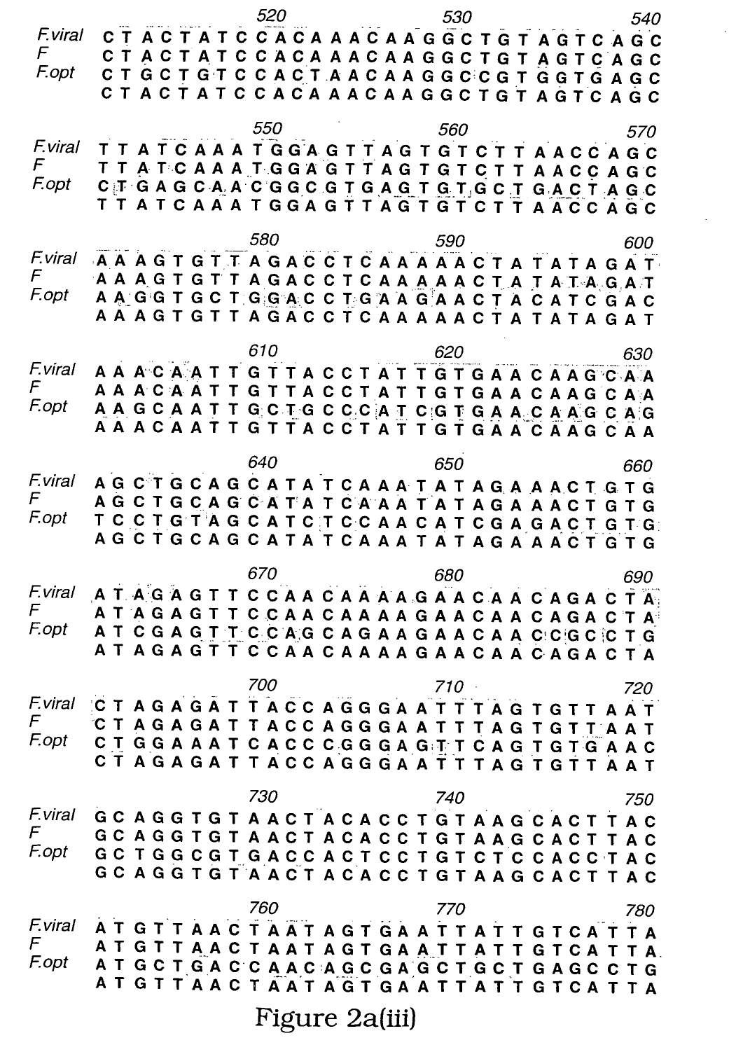

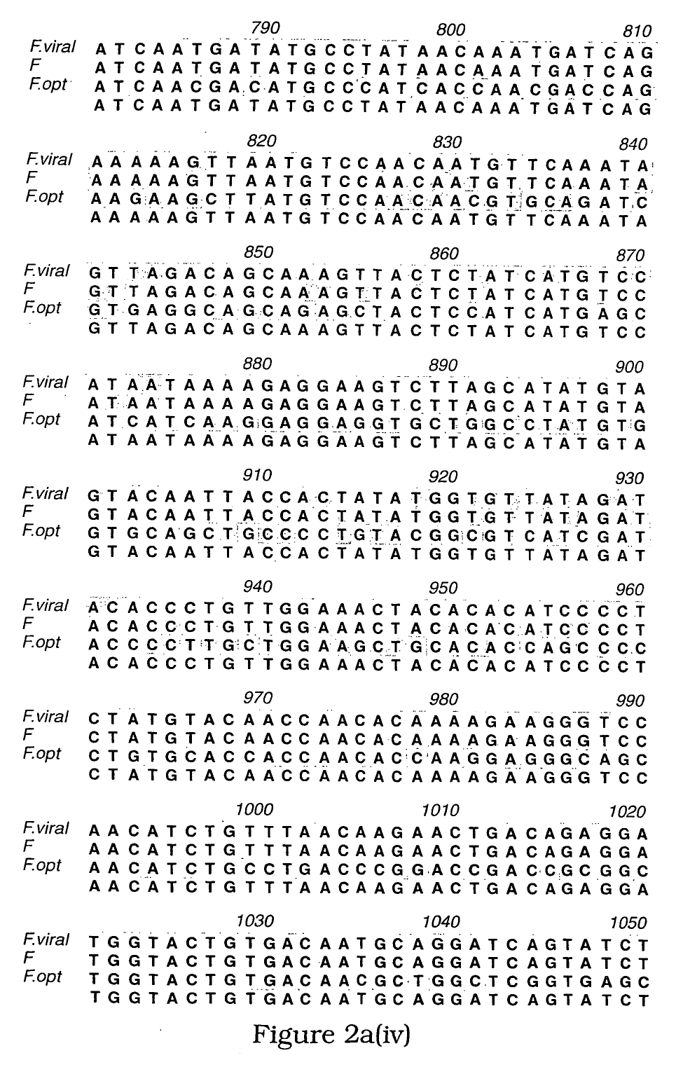

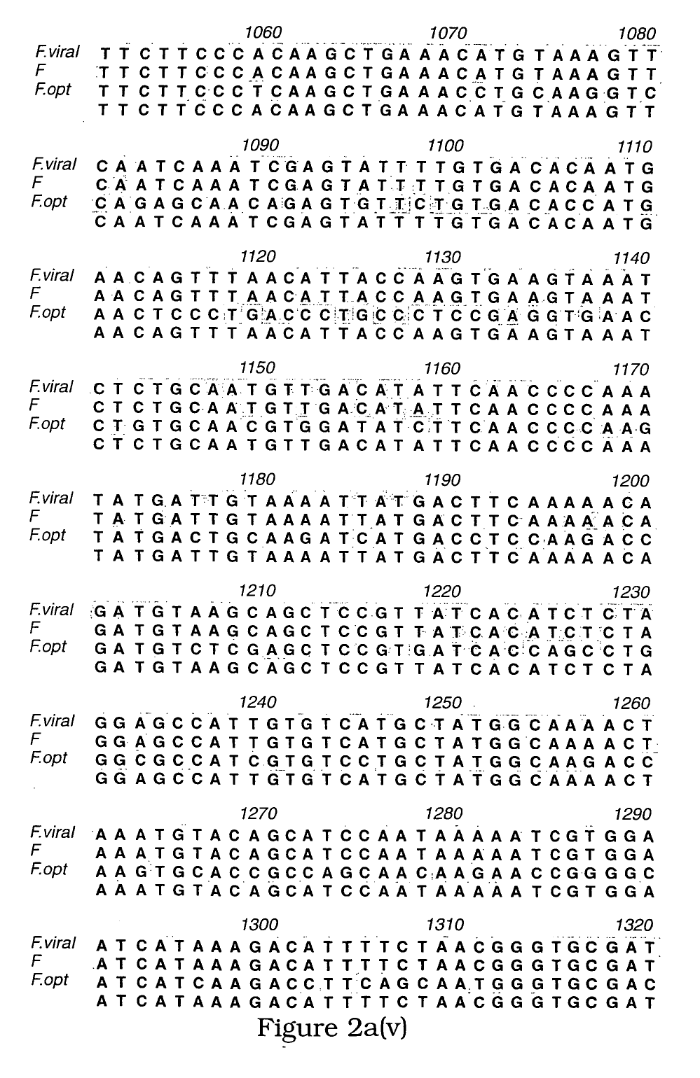

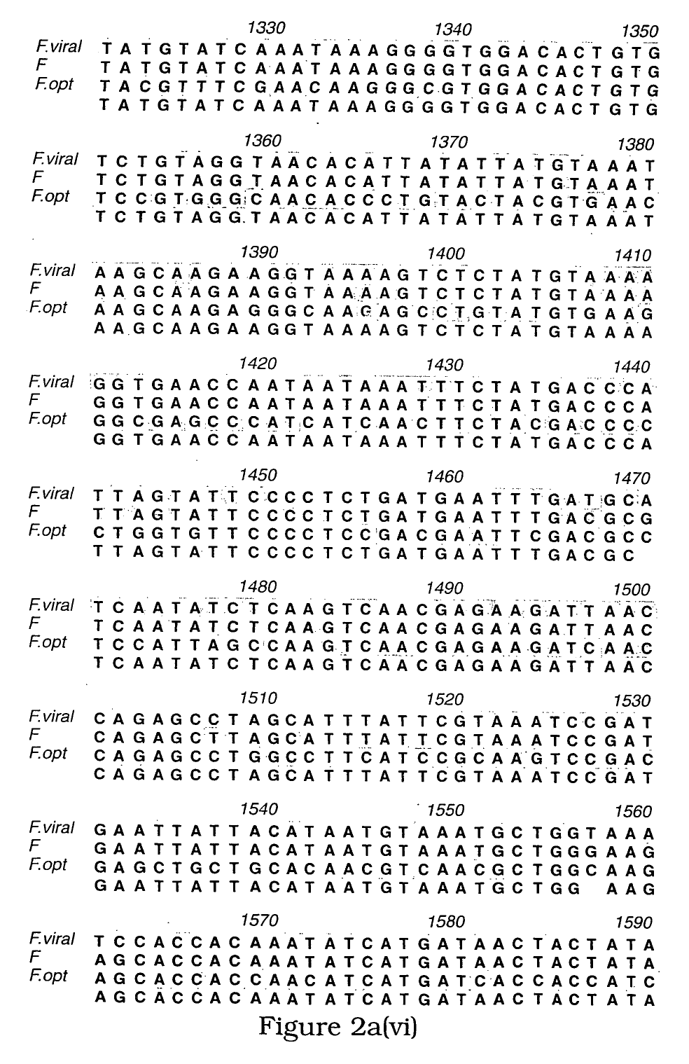

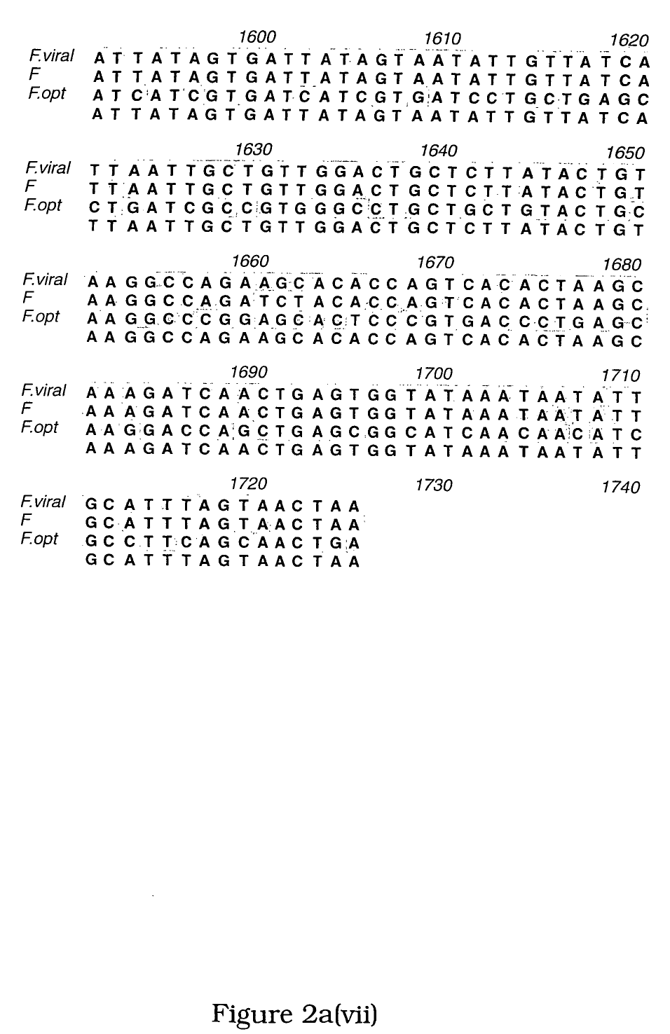

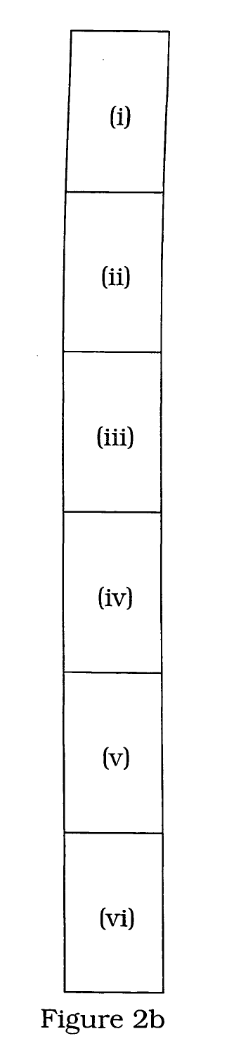

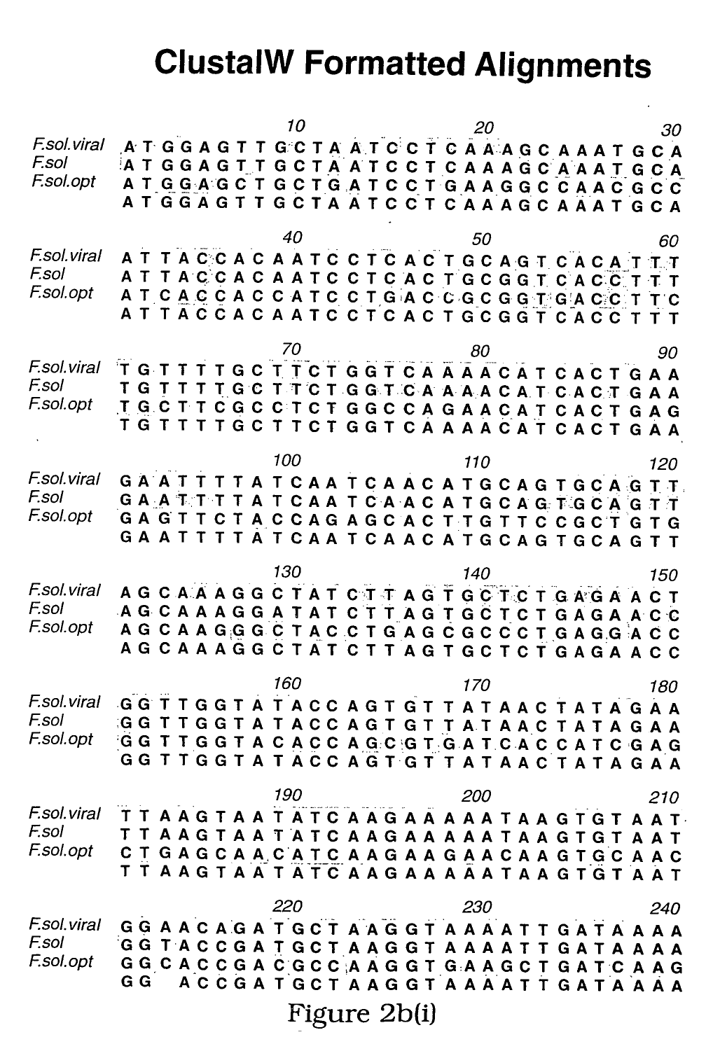

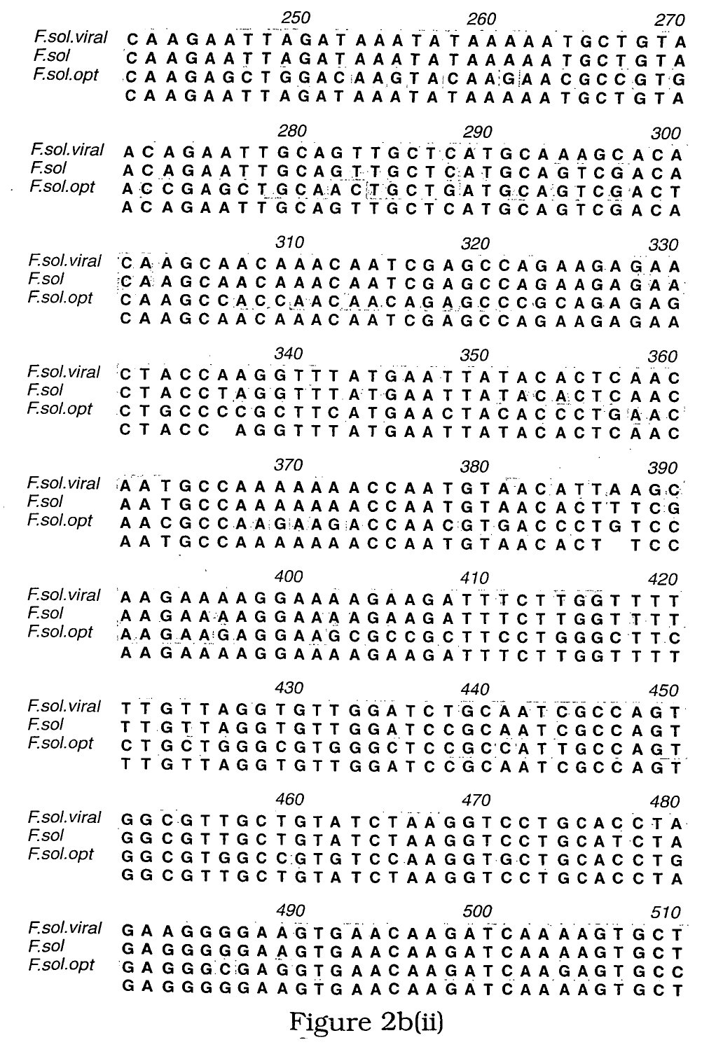

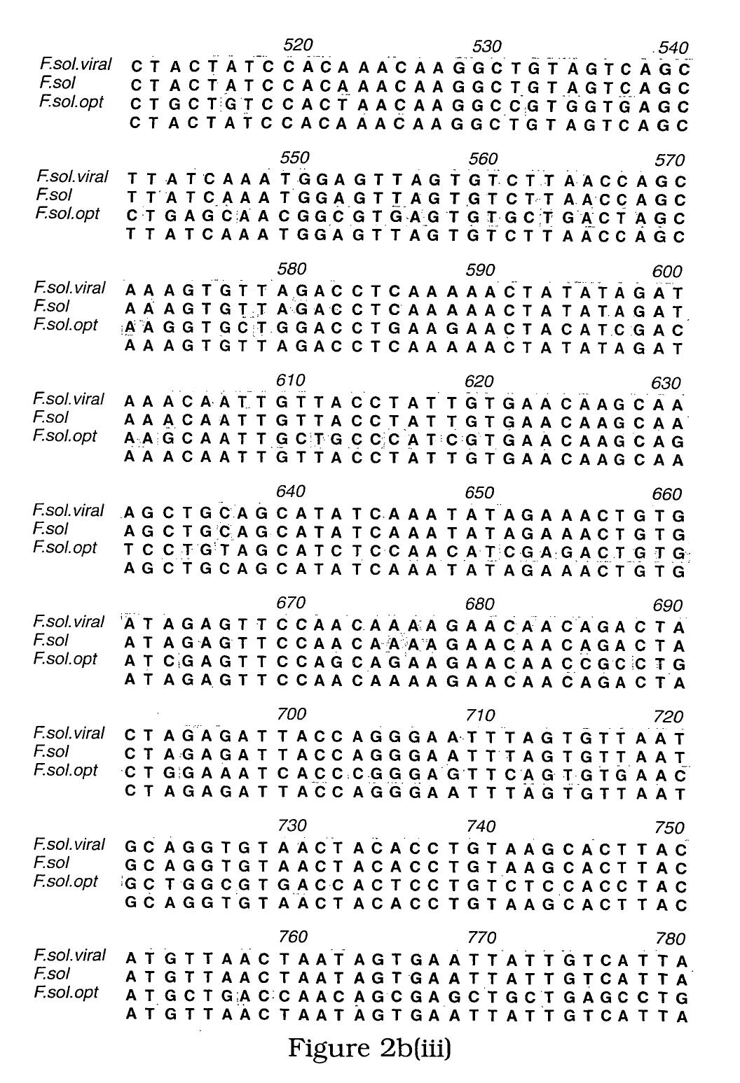

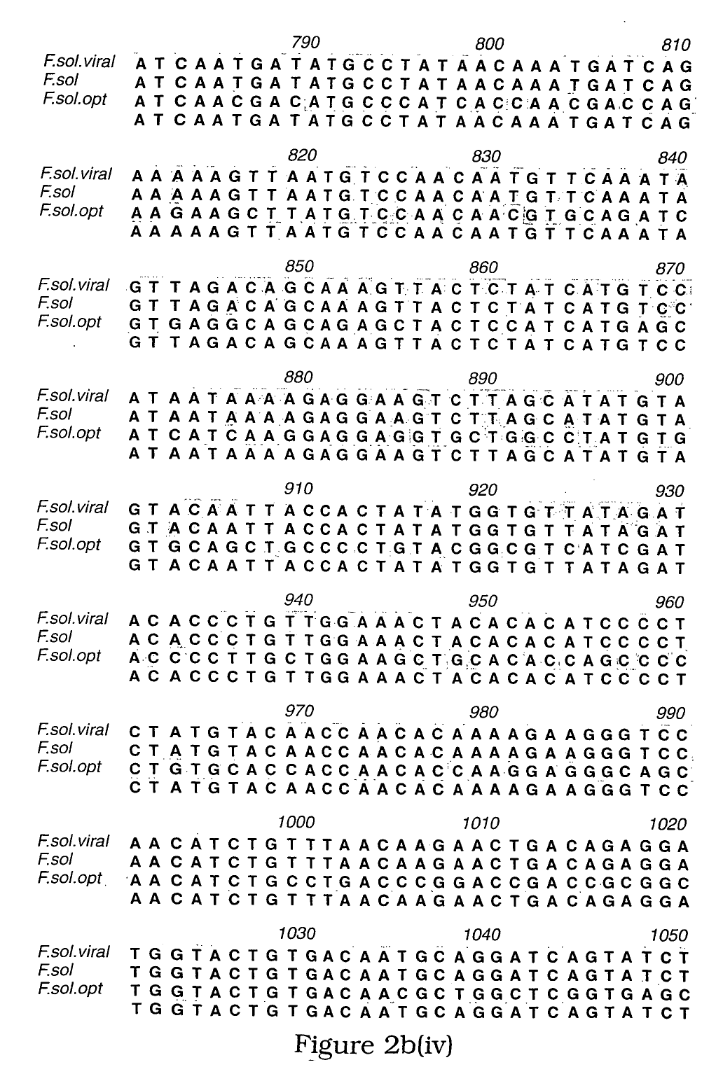

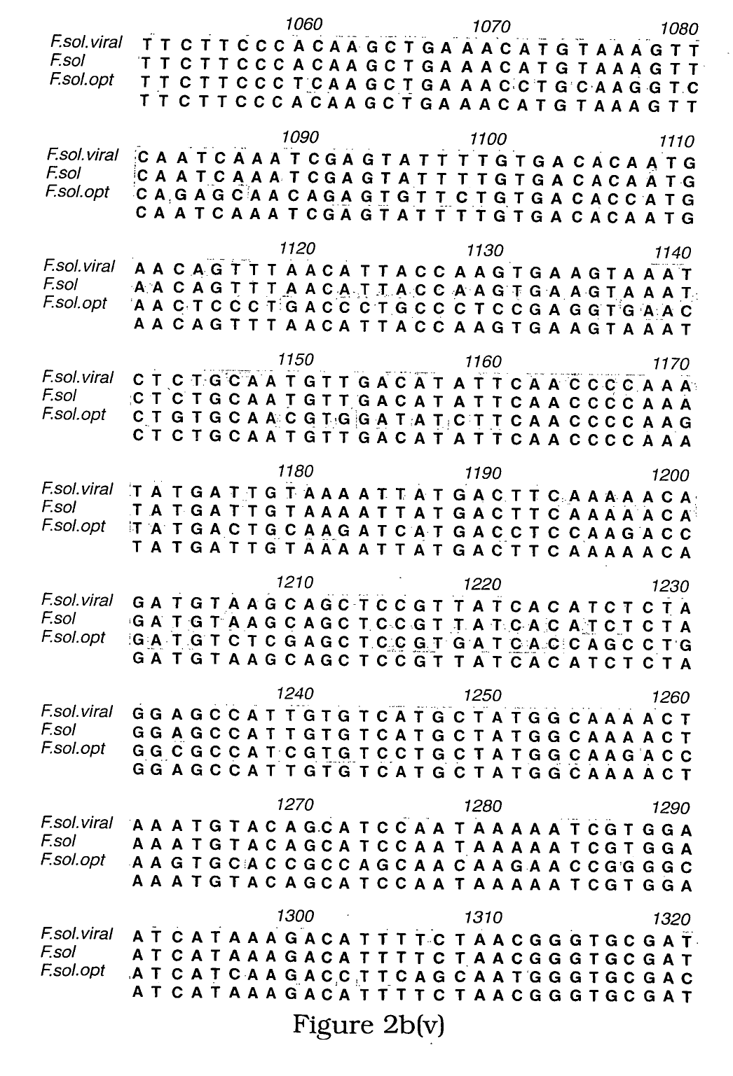

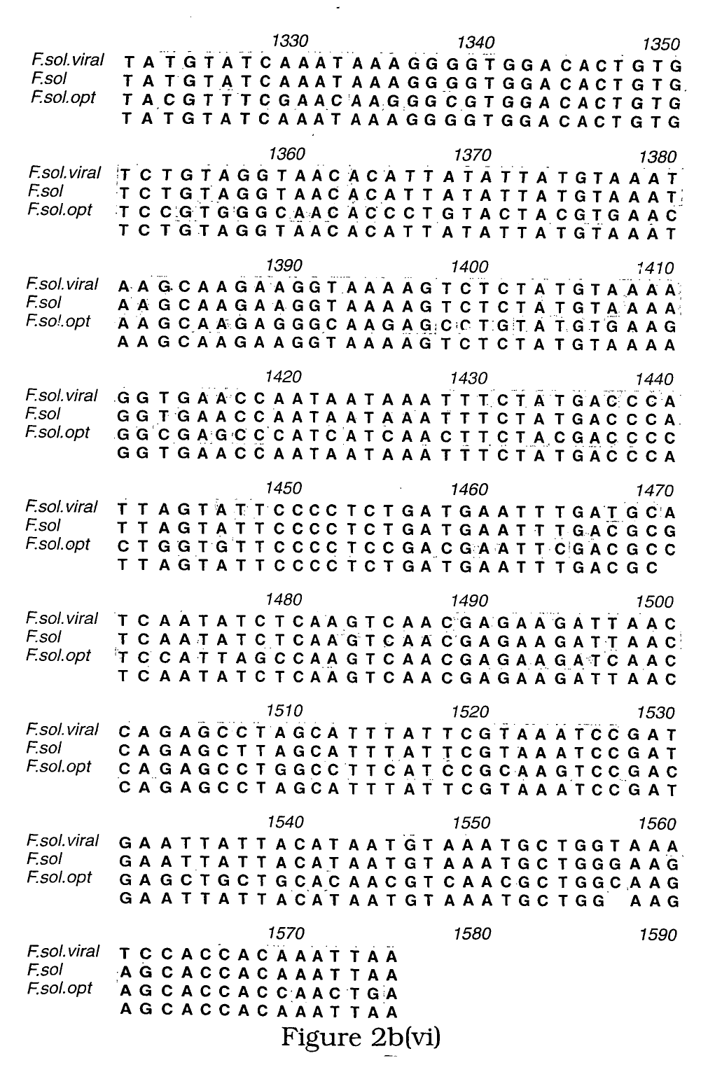

The present invention is exemplified herein with respect to the F, P, N and SH proteins, the naturally occurring encoding nucleic acid sequences of which are defined in<400>1, <400>505, <400>508 and <400>511, respectively. Without limiting the present invention to any one theory or mode of action, the inventors have determined that eukaryotic expression of a viral gene becomes possible where selected A rich and T rich regions of the naturally occurring gene are modified to express increased numbers of G rich and C rich nucleotides. This is achieved by replacing selected A or T nucleotides with a G or C nucleotide, respectively. The resultant modified codon, however, preferably encodes the same amino acid as that encoded by the naturally occurring codon. With respect to the F gene, for example, the codon TTG commences at nucleotide 7 of the naturally occurring respiratory syncytial viral F protein encoding nucleic acid sequence (provided in <400>1). This codon encodes an L amino acid. In the codon optimised F protein encoding nucleic acid sequence, represented herein in FIG. 2a, the codon TTG is modified to read CTG, which modified codon nevertheless encodes the L amino acid. The present invention does not, however, relate to the sequence as published by Kuhnle et al (1998) insofar as the sequence is used for codon optimisation.

The preferred embodiment of the present invention is to optimise the viral protein encoding nucleotide sequence such that the naturally occurring viral protein amino acid sequence or fragment thereof, is produced. However, it should be understood that it is nevertheless within the scope of the present invention to optimise a viral protein encoding nucleotide sequence in terms of expressing increased G plus C content, as required to achieve efficient mammalian host cell expression, despite the fact that an optimised codon may thereafter encode an amino acid different to that originally encoded by the codon which naturally existed at that position. This may occur, for example, where the newly substituted amino acid does not significantly alter the structural and/or functional properties which are required of the recombinantly produced protein. For example, certain conservative amino acid substitutions may not alter functional properties. Similarly, amino acid substitutions in regions outside the protein's functionally active regions may be acceptable in terms of the use to which the expressed protein is to be put.

In terms of optimising the naturally occurring F protein encoding nucleotide sequence, the number of codons which are optimised in any given situation will depend on the object to be achieved. For example, optimisation of between 1 and 10 codons may be sufficient to enable production of a level of eukaryotic host cell expression sufficient for a particular purpose. However, in order to achieve still more efficient levels of expression and/or expression product functional activity, it may be desirable to optimise a larger number of codons. In this regard, in a most preferred embodiment, the optimised F, P, N and SH protein encoding nucleic acid sequences correspond to the sequences defined in<400>5, <400>556, <400>559, and <400>562, respectively. However, it should be understood that the present invention extends to the use of derivatives of these sequences.

By “nucleotide splice site deletion” optimisation is meant that the nucleotide sequence encoding a subject viral protein has been altered to remove one or more potential RNA splice sites. Without limiting the present invention to any one theory or mode of action, it is thought that inefficient expression of nucleotide sequences derived from negative sense single strand RNA viruses is due, in part, to the presence of RNA splice sites in the subject RNAs. These viruses replicate cytoplasmically in the naturally occurring host cell environment. Accordingly, there is a lack of selective pressure against RNA sequences which comprise one or more such splice sites since the enzymes which splice eukaryotic cell RNA are generally only present in the nucleus. However, since the recombinant expression system of the present invention is based, in one embodiment, on the introduction into a eukaryotic host cell of a DNA molecule encoding the viral protein of interest, the requisite synthesis of DNA complementary to the naturally occurring viral RNA gene would consequently also result in copying of any splice sites present in the RNA. Transcription of these DNAs will occur in the nucleus of the eukaryotic host cell thereby exposing RNA transcribed from the subject DNA to the nuclear RNA splicing enzymes of the host cell.

In terms of optimising the naturally occurring viral protein encoding nucleotide sequence, the number of splice sites which are deleted in any given situation would depend on the object to be achieved. For example, if it is desired to produce the full length viral protein, then all splice sites occurring within the protein coding region of the encoding nucleic acid molecule should be deleted. However, if it is desired to produce only a fragment of the subject protein (for example, the Fₛₒₗ portion of the F protein which, as hereinbefore defined, does not comprise the transmembrane and cytoplasmic regions of the F protein) then only the splice sites within that region need be removed.

Deletion of the subject splice sites is preferably achieved by substituting one or more nucleotides which comprise a splice site recognition sequence such that this sequence is no longer recognised by the relevant RNA splicing enzyme. It should be understood, however, that any other suitable method of mutating the splice site may be utilised within the context of the present invention.

The present invention is therefore preferably directed to a method of facilitating production of a protein or derivative thereof from a negative sense single stranded RNA virus, said method comprising expressing in a mammalian host cell a nucleic acid molecule encoding said protein or derivative thereof, the nucleotide sequence of which nucleic acid molecule is optimised for expression by said mammalian host cell wherein said optimisation is codon optimisation and/or nucleotide splice site deletion.

Preferably, the subject protein is a fusion protein (more particularly the F protein), N, P or SH.

Preferably, the negative sense single stranded RNA virus is a virus of the family Paramyxoviridae. More preferably the virus is of the sub-family Pneumovirinae and still more preferably the subject virus is a virus of the genus Pneumovirus. Most preferably, the virus is respiratory syncytial virus.

It should be understood that the present invention extends to the use of derivatives of the optimised nucleic acid sequences.

Most preferably, said codon optimisation comprises modification of at least one A and/or T comprising codon to express G and C, respectively and said mammalian splice site deletion comprises deletion of at least one RNA splice site. To the extent that the nucleic acid molecule which is introduced into the host cell is a DNA molecule, the subject deletion would relate to the region of the DNA molecule which would encode the RNA splice site.

By “derivatives” is meant nucleic acid sequences derived from single or multiple nucleotide substitutions, deletions and/or additions including fusion with other nucleic acid molecules. In accordance with this definition, “derivative” therefore extends to sequences comprising any one or more of the optimised codons and/or optimised splice site regions of <400>5, <400>6, <400>556, <400>559 or <400>562.

Reference to a “derivative” of the subject nucleotide sequence should also be understood to extend to nucleotide sequences comprising nucleic acid substitutions, deletions or additions other than for the purpose of optimising codons. For example, an optimised viral protein encoding nucleotide sequence may additionally comprises endonuclease restriction sites which are not expressed by the naturally occurring counterpart of the subject sequence. These may be incorporated to facilitate the generation of protein mutants. In one preferred embodiment, for example, the subject nucleotide sequence derivative comprises one or more of the endonuclease restriction sites expressed in<400>3 or <400>4.

In terms of a most preferred embodiment of the present invention, <400>1 defines the protein encoding region of the naturally occurring respiratory syncytial virus F protein. <400>3 defines the <400>1 sequence as modified to incorporate endonuclease restriction sites designed to facilitate the generation of protein recombinants. <400>5 defines the F protein encoding nucleotide sequence of <400>3 further modified to minimise the presence of regions which would encode RNA splice sites and to express optimised codons. The amino acid sequence encoded by these nucleotide sequences is provided in <400>7.

Expression of <400>5 in accordance with the method of the present invention will be sought where production of the full length F protein is required. This may occur, for example, where expression of a functional molecule is required for the performance of function based screening assays designed to detect F protein modulatory agents. However, in another embodiment, production of a portion only of the F protein may be desired. For example, production of the Fₛₒₗ portion is particularly desirable for the purpose of 3 dimensional structural analysis, by X-ray crystallography, of the F protein active regions. Furthermore, Fₛₒₗ portion production facilitates the rational identification, modification and design of F protein modulatory agents based on analysing the agent in terms of its physical interaction with the F2 and F1 portions. In this regard, <400>2 defines the protein encoding region of the naturally occurring respiratory syncytial viral Fₛₒₗ portion of the F protein. <400>4 defines the <400>2 sequence as modified to incorporate endonuclease restriction sites designed to facilitate the generation of protein recombinants. <400>6 defines the Fₛₒₗ protein encoding nucleotide sequence of <400>4 further modified to minimise the presence of regions which would encode RNA splice sites and to express optimised codons. The amino acid sequence encoded by these nucleotide sequences is provided in<400>8.

According to this preferred embodiment there is provided a method of facilitating production of F protein or derivative thereof from respiratory syncytial virus, said method comprising expressing in a mammalian host cell a nucleic acid molecule encoding said F protein or derivative thereof, the nucleotide sequence of which nucleic acid molecule is optimised for expression by said mammalian host cell wherein said optimisation is nucleotide splice site deletion.

In another preferred embodiment the present invention is directed to a method of facilitating production of a Fₛₒₗ portion of an F protein or derivative thereof from respiratory syncytial virus, said method comprising expressing in a host cell a nucleic acid molecule encoding said Fₛₒₗ portion or derivative thereof, the nucleotide sequence of which nucleic acid molecule is optimised for expression by said mammalian host cell wherein said optimisation is nucleotide splice site deletion.

In still another preferred embodiment there is provided a method of facilitating production of F protein or derivative thereof from respiratory syncytial virus, said method comprising expressing in a mammalian host cell a nucleic acid molecule encoding said F protein or derivative thereof, the nucleotide sequence of which nucleic acid molecule is optimised for expression by said mammalian host cell wherein said optimisation is codon optimisation.

In yet another preferred embodiment the present invention is directed to a method of facilitating production of a Fsol portion of an F protein or derivative thereof from respiratory syncytial virus, said method comprising expressing in a host cell a nucleic acid molecule encoding said Fₛₒₗ portion or derivative thereof, the nucleotide sequence of which nucleic acid molecule is optimised for expression by said mammalian host cell wherein said optimisation is codon optimisation.

In another preferred embodiment there is provided a method of facilitating production of F protein or derivative thereof from respiratory syncytial virus, said method comprising expressing in a mammalian host cell a nucleic acid molecule encoding said F protein or derivative thereof, the nucleotide sequence of which nucleic acid molecule is optimised for expression by said mammalian host cell wherein said optimisation is nucleotide splice site deletion and codon optimisation.

In still yet a more preferred embodiment, there is provided a method of facilitating the production of a F protein or derivative thereof from a respiratory syncytial virus, said method comprising expressing in a host cell the nucleotide sequence set forth in <400>5 or derivative thereof.

Preferably said nucleotide sequence is substantially as set forth in <400>5.

In another preferred embodiment, there is provided a method of facilitating the production of a Fₛₒₗ portion of an F protein or derivative thereof from respiratory syncytial virus, said method comprising expressing in a host cell the nucleotide sequence set forth in <400>6 or derivative thereof.

Preferably said nucleotide sequence is substantially as set forth in <400>6.

In terms of another most preferred embodiment of the present invention, <400>555 defines the protein encoding region of the naturally occurring respiratory syncytial virus P protein. <400>556 defines the P protein encoding nucleotide sequence of <400>555 as modified to express optimised codons. The amino acid sequence encoded by this nucleotide sequences is provided in<400>554.

According to this preferred embodiment there is provided a method of facilitating production of P protein or derivative thereof from respiratory syncytial virus, said method comprising expressing in a mammalian host cell a nucleic acid molecule encoding said P protein or derivative thereof, the nucleotide sequence of which nucleic acid molecule is optimised for expression by said mammalian host cell wherein said optimisation is codon optimisation.

In still a more preferred embodiment, there is provided a method of facilitating the production of a P protein or derivative thereof from respiratory syncytial virus, said method comprising expressing in a host cell the nucleotide sequence set forth in<400>556 or derivative thereof.

Preferably said nucleotide sequence is substantially as set forth in<400>556.

In terms of yet another most preferred embodiment of the present invention, <400>558 defines the protein encoding region of the naturally occurring respiratory syncytial virus N protein. <400>559 defines the N protein encoding nucleotide sequence of <400>558 as modified to express optimised codons. The amino acid sequence encoded by this nucleotide sequence is provided in<400>557.

According to this preferred embodiment there is provided a method of facilitating production of N protein or derivative thereof from respiratory syncytial virus, said method comprising expressing in a mammalian host cell a nucleic acid molecule encoding said N protein or derivative thereof, the nucleotide sequence of wlich nucleic acid molecule is optimised for expression by said mammalian host cell wherein said optimisation is codon optimisation.

In still a more preferred embodiment, there is provided a method of facilitating the production of a N protein or derivative thereof from respiratory syncytial virus, said method comprising expressing in a host cell the nucleotide sequence set forth in<400>559 or derivative thereof.

Preferably said nucleotide sequence is substantially as set forth in<400>559.

In terms of still yet another most preferred embodiment of the present invention, <400>561 defines the protein encoding region of the naturally occurring respiratory syncytial virus SH protein. <400>562 defines the N protein encoding nucleotide sequence of <400>561 as modified to express optimised codons. The amino acid sequence encoded by this nucleotide sequence is provided in <40>560.

According to this preferred embodiment there is provided a method of facilitating production of SH protein or derivative thereof from respiratory syncytial virus, said method comprising expressing in a mammalian host cell a nucleic acid molecule encoding said SH protein or derivative thereof, the nucleotide sequence of which nucleic acid molecule is optimised for expression by said mammalian host cell wherein said optimisation is codon optimisation.

In still a more preferred embodiment, there is provided a method of facilitating the production of a SH protein or derivative thereof from respiratory syncytial virus, said method comprising expressing in a host cell the nucleotide sequence set forth in <400>569 or derivative thereof.

Preferably said nucleotide sequence is substantially as set forth in <400>562.

In terms of performing the present invention, methods of deriving and recombinantly expressing nucleic acid molecules will be well known to those of skill in the art as will methodology directed to adding, deleting and/or substituting nucleic acids in a given nucleotide sequence.

In another aspect, the present invention should be understood to extend to the optimised nucleic acid molecules described herein and to the expression products derived therefrom.

In yet another aspect, the inventors have surprisingly determined that induction of F protein functional activity requires not one but two proteolytic cleavage events. The occurrence of these two cleavage events results in the excision of a peptide region from the non-fully functional F protein. Prior to the advent of the present invention, it was thought that F protein activation was the result of a single cleavage event which occurred at the F2/F1 junction. Without limiting the invention to any one theory or mode of action, it is thought that the F2 portion of the non-fully functional F protein in fact comprises an intervening sequence of amino acids which spans the region between the newly identified cleavage site and the F2/F1 junction and which is excised in order to facilitate formation of a functional F glycoprotein. This intervening peptide sequence is thought to comprise excess amino acids and up to three glycosylation sites depending on the particular virus strain from which the F protein is derived. Down-regulation or other form of interference with cleavage at the newly identified cleavage site would therefore interfere with the induction of F protein functional activity.

Accordingly, another aspect of the present invention is directed to a method of regulating the functional activity of a viral F protein, which protein in its non-fully functional form comprises an F2 portion linked, bound or otherwise associated with an F1 portion, which F2 portion comprises an intervening peptide sequence, said method comprising modulating cleavage of said intervening peptide sequence wherein excision of at least part of said intervening sequence from said non-fully functional form of said F protein up-regulates F protein functional activity.

Reference to the subject F protein being in a “non-fully functional form” should be understood to mean that the subject F protein exhibits either no functional activity or a lesser degree of functional activity than the fully cleaved F protein, that is, the F protein which has undergone both cleavage events. Accordingly, “up-regulation” of F protein functional activity should be understood to refer to the induction of a degree or range of functional activities greater than that exhibited by the subject F protein in its non-fully cleaved form. In its natural environment, all F proteins are synthesised in a form which comprises a F2 portion located proximally to a F1 portion. The F1 portion region of the F protein comprises a transmembrane region and an intracellular domain (Collins et al, 1996). Reference to a “non-fully functional form” of the F protein should also be understood to extend to forms of the F protein which have undergone only partial cleavage.

For example, the subject non-fully functional form of the F protein may only have undergone cleavage of the previously known cleavage site but not yet at the newly identified cleavage site.

Prior to the advent of the present invention, it was thought that activation of the F protein occurred following cleavage at the F protein site defined by the sequence KKRKRR (<400>563) thereby cleaving the F2 portion of the non-fully functional F protein from the F1 portion. The F1 portion of the F protein is defined, in FIG. 1, as commencing at the F residue which follows the cleavage recognition site KKRKRR. However, the precise location at which this cleavage event occurs is not actually known. Accordingly, it should be understood that the cleavage event may occur either between two residues located proximally to the cleavage recognition site KKRKRR or it may occur between two residues within this site. The definitions of “F2 portion”, “F1 portion” and “F2/F1 junction” as provided herein should therefore be interpreted in light of this understanding.

As detailed above, the inventors have now determined that cleavage at this region alone will not fully activate the F protein. Rather, a second cleavage event must occur at an F protein site distinct from that of the known cleavage site (the known cleavage site being referred to as “site 1”). This second cleavage site is located in the amino terminus direction of the previously known cleavage site and is characterised by expression of the cleavage recognition sequence RARR (<400>564) (herein referred to as “site 2”). When considered in light of the structure of the F protein as it was previously understood (and as depicted in FIG. 1) site 2 is located within the F2 portion of the F protein while the previously known cleavage site is located at the F2/F1 portion junction.

For the purpose of the present invention, it should be understood that the F protein amino acid sequence located in the amino terminus direction of cleavage site 1 is herein referred to as the F2 portion while the amino acid sequence located in the carboxy terminus direction of the cleavage site 1 is herein referred to as the F1 portion. The newly identified cleavage site is therefore located within the F2 portion. The F protein amino acid sequence located between the site 1 and site 2 points of cleavage is herein referred to as the “intervening sequence”. Accordingly, in light of the definition herein provided, the “intervening sequence” forms part of the F2 portion of the non-fully functional form of the F protein. Excision of “at least part of” said intervening sequence should be understood to mean that at least a portion of the sequence which is excised following the two cleavage events is derived from the intervening sequence region as herein defined. However, it should be understood that the excised sequence may also comprise part of the non-intervening sequence region of the F2 and/or F1 portion sequences as herein defined.

Without limiting the present invention to any one theory or mode of action, it is thought that cleavage of the intervening sequence at the two cleavage sites results in complete disassociation of the intervening sequence from the F protein. Accordingly, the term “excision” is intended to encompass complete disassociation of the intervening sequence from the non-fully functional form of the F protein in order to form the functionally active F protein. However this term should also be understood to extend to a cleavage event which does not necessarily result in complete disassociation of at least part of the intervening sequence but leads to a conformational change in the secondary or tertiary structure of the intervening sequence and/or the F2/F1 portions. For example, in some circumstances an appropriate conformational shift in the intervening sequence relative to the F2 and F1 portions may be sufficient to achieve some up-regulation of the functional activity of the F protein. It should also be understood that the two cleavage events may occur concurrently in order to effect excision. Alternatively, the cleavage events may occur consecutively. For example, cleavage at site 1 may occur initially, followed by cleavage at site 2 (and hence formation of the fully functional form of the F protein) at a subsequent point in time. The present invention should also be understood to extend to a sequence of cleavage events commencing with cleavage at site 2.

The present invention is exemplified with respect to respiratory syncytial virus F protein. The respiratory syncytial virus F protein amino acid sequence is defined <400>7. In accordance with the amino acid sequence numbering provided in<400>7, the previously known cleavage site is located at the region of the F protein defined by the amino acid sequence KKRKRR, which sequence spans amino acid numbers 131 to 136 of <400>7.

The second cleavage point, which has been identified by the present inventors, is localised to the region of the F protein defined by tie amino acid sequence RARR, which sequence spans amino acid numbers 106-109 of <400>7.

In a preferred embodiment the present invention is directed to a method of regulating the functional activity of a Paramyxoviridae derived F protein, which protein in its non-fully functional form comprises an F2 portion linked, bound or otherwise associated with an F1 portion, which F2 portion comprises an intervening peptide sequence, said method comprising modulating cleavage of said intervening peptide sequence wherein excision of at least part of said intervening sequence from said non-fully functional form of said F protein up-regulates F protein functional activity.

Still more preferably said F protein is derived from the Genus Pneumovirus and still more preferably said virus is respiratory syncytial virus.

In a most preferred embodiment there is provided a method of regulating the functional activity of a respiratory syncytial virus F protein, which protein in its non-fully functional form comprises an F2 portion linked, bound or otherwise associated with an F1 portion, which F2 portion comprises an intervening peptide sequence, said method comprising modulating cleavage of said intervening peptide sequence, wherein excision of at least part of said intervening sequence from said non-fully functional form of said F protein up-regulates F protein functional activity and wherein said cleavage events occur at the cleavage sites defined by the peptide sequences RARR (<400>564) and KKRKRR (<400>563).

That the subject cleavage events “occur at” a given cleavage site should be understood to mean that cleavage of the F protein amino acid sequence will involve cleavage of the bonding mechanism associated with anyone or more of the amino acids comprising the defined sites. Without limiting the invention in any way, the amino acids comprising the cleavage sites define the peptide sequence recognised by the proteolytic enzyme which cleaves the subject F protein (Steiner, 1998).

In a related aspect, the present invention provides a method of regulating the functional activity of a viral F protein, which protein in its non-fully functional form comprises the structure:

X₁, X₂, X₃

wherein:

X₁ comprises the non-intervening peptide sequence region of the F2 portion;

X₂ comprises the intervening peptide sequence region of the F₂ portion; and

X₃ comprises the F1 portion

said method comprising modulating cleavage of said intervening peptide sequence wherein excision of at least part of said intervening sequence from said non-fully functional form of said F protein up-regulates F protein functional activity.

The representation X₁, X₂, X₃ is not to be taken as imposing any sequential constraints on the subject F protein and the present invention encompasses any conformational secondary and/or tertiary structural arrangement of X₁, X₂, X₃ to the extent that X₁ and X₃ are both linked, bound or otherwise associated with X₂ in the subject F protein's non-fully functional form.

Reference to the “non-intervening peptide sequence region” of F2 should be understood as a reference to that part of the F2 subunit which does not form part of the intervening sequence as herein defined.

Preferably said virus is a virus from the family Paramyxoviridae and still more preferably is a virus of the Genus Pneumovirus. Most preferably said virus is respiratory syncytial virus.

In another preferred embodiment said cleavage events occur at the cleavage sites comprising X₂ and defined by the peptide sequences RARR (<400>564) and KKRKRR (<400>563).

Modulating cleavage of the intervening sequence can be achieved by any one of a number of methods including, but in no way limited to:

(i) Contacting the F protein or F protein encoding nucleic acid molecule with a proteinaceous or non-proteinaceous molecule (herein referred to as an “agent”) which up-regulates or down-regulates cleavage of either one or both of the cleavage sites comprising the intervening sequence. The proteinaceous or non-proteinaceous molecule may achieve this objective by functioning as either an agonist or antagonist of the cleavage event, for example. This molecule may act in any one of a number of ways including interacting with the subject F protein or interacting with the enzymes which recognise the cleavage sites comprising the F protein.

(ii) Mutating the amino acid sequence of the F protein cleavage site such that proteolytic cleavage cannot occur. This can be performed at either the amino acid sequence level (for example by adding, substituting or deleting an amino acid in the newly identified cleavage site) or at the nucleotide level such that the transcribed and translated F protein expression product does not express a functional form of the subject cleavage site.

Said proteinaceous molecule may be derived from natural or recombinant sources including fusion proteins or following, for example, natural product screening. Said non-proteinaceous molecule may be, for example, a nucleic acid molecule or may be derived from natural sources, such as for example natural product screening or may be chemically synthesised. The present invention contemplates chemical analogues of the F protein capable of acting as agonists or antagonists of either the fully functional or non-fully functional F protein. Chemical agonists may not necessarily be derived from the F protein but may share certain conformational similarities. Alternatively, chemical agonists may be specifically designed to mimic certain physiochemical properties of the F protein. Antagonists may be any compound capable of blocking, inhibiting or otherwise preventing F protein from carrying out its normal biological function. Antagonists include monoclonal antibodies specific for the F protein, or parts of the F protein, and antisense nucleic acids which prevent transcription and/or translation of the F protein encoding nucleic acid molecule or mRNA in mammalian cells.

Although the preferred method is to inhibit, retard or otherwise down-regulate F protein functional activity by preventing cleavage of the non-fully functional F protein form and subsequent activation, up-regulation of F protein functional activity may be desired in certain circumstances. In this regard, use of agonistic agents which augment and/or induce the cleavage events herein described may be utilised. Reference to “down-regulating” F protein functional activity should be understood to encompass prevention of the functional activation of the non-fully functional F protein.

Accordingly, in a most preferred embodiment there is provided a method of inhibiting, retarding or otherwise down-regulating the functional activity of a Paramyxoviridae derived F protein, which protein in its non-fully functional form comprises an F2 portion linked, bound or otherwise associated with an F1 portion, which F2 portion comprises an intervening peptide sequence, said method comprising inhibiting or otherwise down-regulating cleavage of said intervening peptide sequence.

Preferably said F protein is derived from the Genus Pneumovirus and still more preferably said virus is respiratory syncytial virus. Most preferably said cleavage events occur at the cleavage sites defined by peptide sequences RARR (<400>564) and KKRKRR (<400>563).

In another most preferred embodiment the present invention provides a method of down-regulating the functional activity of a Paramyxoviradae derived F protein, which protein in its non-fully functional form comprises the structure:

X₁X₂X₃

wherein:

X₁ comprises the non-intervening peptide sequence region of the F2 portion;

X₂ comprises the intervening peptide sequence region of the F2 portion; and

X₃ comprises the F1 portion

said method comprising inhibiting or otherwise down-regulating cleavage of said intervening peptide sequence.

Preferably said F protein is derived from the Genus Pneumovirus and still more preferably said virus is respiratory syncytial virus. Most preferably said cleavage events occur at the cleavage sites defined by peptide sequences RARR (<400>564) and KKRKRR (<400>563).

Without limiting the present invention to any one theory or mode of action, the F proteins of viruses of the family Paramyxoviridae are involved in facilitating fusion between the virus envelope and the host cell plasma membrane in order to effect infection. Further, it is thought that the F proteins are also inserted into the host plasma membrane where, during maturation, the virions bud through the region of the membrane containing these proteins. Accordingly, it is thought that down-regulating F protein functional activity will inhibit or otherwise reduce virion fusion with and infection of a potential host cell and/or virion budding. Accordingly, the development of a method for recombinantly expressing the F protein by eukaryotic cells, and in particular mammalian cells, now facilitates the development of screening assays, utilising the F protein produced in accordance with the method of the present invention, for the purpose of identifying agents capable of modulating F protein functional activity, and preferably, down-regulating F protein functional activity.

Screening for agents which modulate F protein functional activity can be achieved by any one of a number of suitable methods, which would be known to those of skill in the art, including but not limited to:

(i) High throughput screening for agents which modulate F protein functional activities utilising assays based on the detection of changes in F protein functioning. Such changes may be detected directly or indirectly.

An example of indirect detection of modulation of F protein functioning includes the screening of agents on cultured cells which have been co-transfected with the F protein encoding nucleic acid molecule of the present invention and a virus which utilises the F protein in order to propagate. In this regard, either the full length F protein encoding nucleic acid sequence can be utilised or a partial sequence which encodes a functionally active F protein portion can be used. By assessing cell viability it can be determined whether the subject agent inhibits or down-regulates F protein functioning thereby preventing F protein mediated propagation of cell to cell fusion. This would be evident by continued cell viability. A typical assay of this type can be performed, for example, in 293 cells which have been transiently co-transfected with a plasmid encoding the adenoviral VA RNA genes.

(ii) Antibody Recognitior₁ Assays

The use of antibodies which bind to conformational epitopes is a recognised method for assessing whether a protein's three dimensional structure differs from the natural state. Thus an assay can be conducted on protein exposed to agents that are expected to modulate function via perturbation of the native conformation or interference with a functional conformational transition. A number of suitable F-specific antibodies and their target sites have been identified by workers in the field (see for example Lopez et al., 1998 and references therein). For example, F protein exposed to agents intended to modulate F function is subsequently incubated with F specific monoclonal antibodies using an ELISA format. Reduction or increase in F binding relative to F which has not been exposed to agents is measured by addition of polyclonal antibody to F followed by suitable detection reagents according to standard methods.

(iii) Immunisation leading to protection and/or virus neutralisation

RSV is known to infect a wide range of animal species when inoculated experimentally into the respiratory tract and several small animal experimental models have been described (see for example Collins et al., 1996 and references therein). These models can be used to determine whether immunisation is protective and/or results in the production of a virus neutralising response.

An example of a suitable method is as follows: Cotton rats (average weight 100 g) are anesthetized with methoxyflurane and a sample of pre-immune blood harvested via standard procedures. While anesthetized, the cotton rats are administered a suitable quantity of agent (for example, purified F protein) via an appropriate route (for example, intramuscular injection or intranasal instillation). The cotton rats are housed for an appropriate period (generally several days to weeks depending on the agents under consideration and the objectives of the study) and then anesthetized as above. Anesthetized animals are bled to obtain a post-immunization sample and infected with 100,000 plaque forming units of a suitable RSV strain (for example, RSV Long). Four days later the animals are sacrificed and lungs harvested aseptically. Protective efficacy of the agent is measured by determination of the effect on whole lung virus titre. Briefly, lungs are homogenised in sterile saline (1:10 w/v) and virus concentration determined by standard methods (for example, plaque assay).

To determine whether the agent elicited a neutralising response, pre-immunization and post-immunization samples and control samples are examined using a virus neutralization test. An example of such a test is as follows. Sera are prepared from the blood samples according to standard methods. Serial dilutions of the sera are then prepared and mixed with a known concentration of RSV (for example, 100 plaque forming units of RSV Long). Mixtures are incubated for 1 hour at room temperature before being assayed for virus concentration by standard methods (for example, plaque assay). A neutralizing response is characterised by reduction in virus titre in comparison to control samples.

Accordingly, in another aspect there is provided a method for detecting an agent capable of regulating the functional activity of a viral F protein or derivative thereof said method comprising contacting a eukaryotic cell expressing an optimised nucleic acid molecule encoding said viral F protein or derivative thereof, as hereinbefore described, with a putative modulatory agent and detecting an altered expression phenotype and/or functional activity.

It should be understood that the subject agent may act via any mechanism including, but not limited to, modulating the cleavage events hereinbefore described.

In yet another aspect there is provided a method for detecting an agent capable of regulating the functional activity of a viral F protein or derivative thereof said method comprising contacting a host cell, which host cell expresses a nucleic acid molecule encoding the non-fully functional form of said viral F protein or derivate thereof as hereinbefore described, with a putative modulatory agent and detecting an altered expression phenotype and/or altered functional activity wherein said agent modulates cleavage of the intervening peptide sequence.

To the extent that this aspect of the present invention is directed to screening for agents which modulate the site 2 cleavage event, it should be understood that this methodology is not limited to systems expressing an optimised nucleic acid sequence but extends to systems utilising any method of expressing the subject F protein.

Reference to a “modulatory agent” should be understood as a reference to an agent which down-regulates, up regulates or otherwise alters at least one functional activity of the subject F protein. For example, the agent may increase or decrease the level of activity of the F protein or it may entirely inhibit F protein functioning. Although the preferred method is to identify agents which inhibit F protein functional activity, for example by preventing cleavage of the non-fully functional form of the F protein, thereby providing a potential antiviral therapy, the identification of agents which up regulate F protein functional activity may be desired in certain circumstances. For example, it is thought that an agent which causes the F protein to prematurely initiate the conformational changes required for fusion would be inactivating.

Still more preferably, said viral F protein is a Pneumovirus F protein and yet still more preferably a respiratory syncytial virus F protein. Most preferably, said codon optimised nucleic acid molecule is the nucleic acid molecule defined in <400>5.

Preferably, said regulation is inhibition, retardation or other form of down-regulation.

Reference to “functional activity” should be understood as a reference to any one or more of the functions which the F protein performs. Accordingly, an agent which modulates the functional activity of the F protein may modulate all or only some of the functions which the F protein performs. The phrase “functional activity” should be understood to include within its scope the cleavage events which the F protein undergoes.

In addition to screening for agents which modulate F protein functional activity utilising function based assays of the type described above, the development of methodology which facilitates production of the F protein or derivatives thereof also facilitates the screening, analysis, rational design and/or modification of agents for modulating F protein functional activity based on analysis of the physical interaction of a putative agent or lead compound with the subject F protein or derivative thereof.

Specifically, in vitro production of the F protein or derivative thereof, which is now possible in light of the development of the present invention, now facilitates analysis of the tertiary structure of the F protein by techniques such as X-ray crystallography. Of particular value in this regard is the fact that the present invention permits production of useful quantities of the F protein Fₛₒₗ portion.

Accordingly, another aspect of the present invention is directed to a method for analysing, designing and/or modifying an agent capable of interacting with a viral F protein or derivative thereof and modulating at least one functional activity associated with said protein, which protein is produced in accordance with the method of the present invention said method comprising contacting said F protein or derivate thereof with a putative agent and assessing the degree of interactive complementarity of said agent with said protein.

Preferably said viral F protein is a Pneumovirus F protein and even more preferably the Fₛₒₗ portion of said Pneumovirus F protein. Still more preferably, said Fₛₒₗ portion is defined by the amino acid sequence of <400>8.

It should be understood that the F protein which is contacted with the putative agent for evaluation of interactive complementarity may be recombinantly produced. However, it should also be understood that the subject protein may take the form of an image based on the structure elucidated via analysis of the F protein produced in accordance with the method of the present invention, such as an electron density map, molecular models (including, but not limited to, stick, ball and stick, space filling or surface representation models) or other digital or non-digital surface representation models or image, which facilitates the analysis of F protein: agent interactions utilising-techniques and software which would be known to those of skill in the art. For example, interaction analyses can be performed utilising techniques such as Biacore real-time analysis of on and off-rates and dissociation constants for binding of ligands (Gardsvoll et al, 1999; Hoyer-Hansen et al, 1997; Ploug, 1998; Ploug et al, 1994; 1995; 1998) and NMR perturbation studies (Stephens et al, 1992).

Reference to “assessing the degree of interactive complementarity” of an agent with the subject F protein should be understood as a reference to elucidating any feature of interest including, but not limited to, the nature and/or degree of interaction between the subject F protein and an agent of interest. As detailed above, any suitable technique can be utilised. Such techniques would be known to the person of skill in the art and can be utilised in this regard. In terms of the nature of the subject interaction, it may be desirable to assess the types of interactive mechanisms which occur between specific residues of any given agent and those of the F protein (for example, peptide bonding or formation of hydrogen bonds, ionic bonds, van der Waals forces, etc.) and/or their relative strengths. It may also be desirable to assess the degree of interaction which occurs between an agent of interest and the subject F protein. For example, by analysing the location of actual sites of interaction between the subject agent and F protein it is possible to determine the quality of fit of the agent into any region of the F protein and the relative strength and stability of that binding interaction. For example, if it is the object that F protein functioning be blocked, an agent which interacts with the F protein such that it blocks or otherwise hinders (for example, sterically hinders or chemically or electrostatically repels) F2/F1 cleavage will be sought. The form of association which is required in relation to modulating F protein functioning may not involve the formation of any interactive bonding mechanism, as this is traditionally understood, but may involve a non-bonding mechanism such as the proximal location of a region of the agent relative to the subject binding region of the F protein, for example, to effect steric hindrance with respect to the binding of an activating molecule. Where the interaction takes the form of hindrance or the creation of other repulsive forces, this should nevertheless be understood as a form of “interaction” despite the lack of formation of any of the traditional forms of bonding mechanisms.

It should also be understood that the F protein which is utilised either in a physical form or as an image, as hereinbefore discussed, to assess the interactive complementarity of a putative agent may be a naturally occurring form of the F protein or it may be a derivative, homologue, analogue, mutant, fragment or equivalent thereof. The derivative, homologue, analogue, mutant, fragment or equivalent thereof may take either a physical or non-physical (such as an image) form.

The determination of F protein binding regions has been made possible only by development of the present invention which has permitted F protein production and thereby has facilitated determination of the three dimensional structure of the F protein and the identification and/or rational modification and design of agents which can be used to modulate F protein functioning.

Without limiting the application of the present invention in any way, the method of the present invention facilitates the analysis, design and/or modification of agents capable of interacting with the F protein. In this regard, reference to “analysis, design and/or modification” of an agent should be understood in its broadest sense to include:

(i) Randomly screening (for example, utilising routine high-throughput screening technology) to identify agents which exhibit some modulatory capacity with respect to F protein functional activity and then analysing the precise nature and magnitude of the agent's modulatory capacity utilising the method of this aspect of the present invention. In this regard, existing crystals could be soaked with said agents or co-crystalisation could be performed. A combination of modelling and synthetic modification of the local compound together with mutagenesis of the F protein could then be performed for example. In screening for agents which may modulate activity, standard methods of phage display and also combinatorial chemistry may be utilised (Goodson et al., 1994; Terrett., 2000). Such interaction studies can also be furthered utilising techniques such as the Biacore analysis and NMR perturbation studies. Such agents are often commonly referred to as “lead” agents in terms of the random screening of proteinaceous or non-proteinaceous molecules for their capacity to function either agonistically or antagonistically. Further, for example, binding affinity and specificity could be enhanced by modifying lead agents to maximise interactions with the F protein. Such analyses would facilitate the selection of agents which are the most suitable for a given purpose. In this way, the selection step is based not only on ill vitro data but also on a technical analysis of sites of agent: F protein interaction in terms of their frequency, stability and suitability for a given purpose. For example, such analysis may reveal that what appears to be an acceptable in vitro activity in respect of a randomly identified agent is in fact induced by a highly unstable interaction due to the presence of proximally located agent: F protein sites which exhibit significant repulsive forces thereby de-stabilising the overall interaction between the agent and the F protein. This would then facilitate the selection of another prospective lead compound, exhibiting an equivalent degree of in vitro activity, but which agent does not, upon further analysis, involve the existence of such de-stabilising repulsive forces.

Screening for the modulatory agents herein defined can be achieved by any one of several suitable methods, including in silico methods, which would be well known to those of skill in the art and which are, for example, routinely used to randomly screen proteinaceous and non-proteinaceous molecules for the purpose of identifying lead compounds.

These methods provide a mechanism for performing high throughput screening of putative modulatory agents such as the proteinaceous or non-proteinaceous agents comprising synthetic, recombinant, chemical and natural libraries.

(ii) The candidate or lead agent (for example, the agent identified in accordance with the methodology described in relation to point (i)) could be modified in order to maximise desired interactions (for example, binding affinity to specificity) with the F protein and to minimise undesirable interactions (such as repulsive or otherwise de-stabilising interactions). Such modification is only possible in light of knowledge of the three-dimensional structure of the F protein and the capacity therefore to identify regions of functional importance, thereby facilitating the structural modification of an agent to maximise an agonistic or antagonistic interaction. Such methodology is particularly applicable to rational drug design.

Methods of modification of a candidate or lead agent in accordance with the purpose as defined herein would be well lcnown to those of skill in the art. For example, a molecular replacement program such as Amore (Navaza, 1994) may be utilised in this regard. The method of the present invention also facilitates the mutagenesis of known signal inducing agents in order to ablate or improve signalling activity.

(iii) In addition to analysing fit and/or structurally modifying existing molecules, the method of the present invention also facilitates the rational design and synthesis of an agent, such as an agonistic or antagonistic agent, based on theoretically modelling an agent exhibiting the desired F protein interactive structural features followed by the synthesis and testing of the subject agent.

It should be understood that any one or more of applications (i)-(iii) above, may be utilised in identifying a particular agent.

In a related aspect, the present invention should be understood to extend to the agents identified utilising any of the methods hereinbefore defined. In this regard, reference to an agent should be understood as a reference to any proteinaceous or non-proteinaceous molecule which modulates at least one F protein functional activity. As hereinbefore discussed, to the extent that the present invention encompasses methods of screening for agents utilising F proteins produced in accordance with the expression system hereinbefore defined, this is not to be taken as a restriction on the methodology which is employed to screen for agents which modulate the newly identified cleavage event. In this regard, the present invention extends to agents identified utilising F protein molecules or derivatives thereof howsoever produced.

Accordingly, the present invention is directed to an agent capable of interacting with a viral F protein and modulating at least one functional activity associated with said viral protein.

Preferably, said agent is identified in accordance with the methods hereinbefore defined.

More preferably, said agent is an antagonist which interacts with a sequence selected from:

| | | | |
|---|---|---|---|
| CFASGQNITE | <400>9 | FASGQNITEE | <400>10 |
| ASGQNITEEF | <400>11 | SGQNITEEFY | <400>12 |
| GQNITEEFYQ | <400>13 | QNITEEFYQS | <400>14 |
| NITEEFYQST | <400>15 | ITEEFYQSTC | <400>16 |
| TEEFYQSTCS | <400>17 | EEFYQSTCSA | <400>18 |
| EFYQSTCSAV | <400>19 | FYQSTCSAVS | <400>20 |
| YQSTCSAVSK | <400>21 | QSTCSAVSKG | <400>22 |
| STCSAVSKGY | <400>23 | TCSAVSKGYL | <400>24 |
| CSAVSKGYLS | <400>25 | SAVSKGYLSA | <400>26 |
| AVSKGYLSAL | <400>27 | VSKGYLSALR | <400>28 |
| SKGYLSALRT | <400>29 | KGYLSALRTG | <400>30 |
| GYLSALRTGW | <400>31 | YLSALRTGWY | <400>32 |
| LSALRTGWYT | <400>33 | SALRTGWYTS | <400>34 |
| ALRTGWYTSV | <400>35 | LRTGWYTSVI | <400>36 |
| RTGWYTSVIT | <400>37 | TGWYTSVITI | <400>38 |
| GWYTSVITIE | <400>39 | WYTSVITIEL | <400>40 |
| YTSVITIELS | <400>41 | TSVITIELSN | <400>42 |
| SVITIELSNI | <400>43 | VITIELSNIK | <400>44 |
| ITIELSNIKK | <400>45 | TIELSNIKKN | <400>46 |
| IELSNIKKNK | <400>47 | ELSNIKKNKC | <400>48 |
| LSNIKKNKCN | <400>49 | SNIKKNKCNG | <400>50 |
| NIKKNKCNGT | <400>51 | IKKNKCNGTD | <400>52 |
| KKNKCNGTDA | <400>53 | KNKCNGTDAK | <400>54 |
| NKCNGTDAKV | <400>55 | KCNGTDAKVK | <400>56 |
| CNGTDAKVKL | <400>57 | NGTDAKVKLI | <400>58 |
| GTDAKVKLIK | <400>59 | TDAKVKLIKQ | <400>60 |
| DAKVKLIKQE | <400>61 | AKVKLIKQEL | <400>62 |
| KVKLIKQELD | <400>63 | VKLIKQELDK | <400>64 |
| KLIKQELDKY | <400>65 | LIKQELDKYK | <400>66 |
| IKQELDKYKN | <400>67 | KQELDKYKNA | <400>68 |
| QELDKYKNAV | <400>69 | ELDKYKNAVT | <400>70 |
| LDKYKNAVTE | <400>71 | DKYKNAVTEL | <400>72 |
| KYKNAVTELQ | <400>73 | YKNAVTELQL | <400>74 |
| KNAVTELQLL | <400>75 | NAVTELQLLM | <400>76 |
| AVTELQLLMQ | <400>77 | VTELQLLMQS | <400>78 |
| TELQLLMQST | <400>79 | ELQLLMQSTQ | <400>80 |
| LQLLMQSTQA | <400>81 | QLLMQSTQAT | <400>82 |
| LLMQSTQATN | <400>83 | LMQSTQATNN | <400>84 |
| MQSTQATNNR | <400>85 | QSTQATNNRA | <400>86 |
| STQATNNRAR | <400>87 | TQATNNRARR | <400>88 |
| QATNNPARRE | <400>89 | ATNNPARREL | <400>90 |
| TNNRARRELP | <400>91 | NNRARRELPR | <400>92 |
| NRARRELPRF | <400>93 | RARRELPRFM | <400>94 |
| ARRELPRFMN | <400>95 | RRELPRFMNY | <400>96 |
| RELPRFMNYT | <400>97 | ELPRFMNYTL | <400>98 |
| LPRFMNYTLN | <400>99 | PRFMNYTLNN | <400>100 |
| RFMNYTLNNA | <400>101 | FMNYTLNNAK | <400>102 |
| MNYTLNNAKK | <400>103 | NYTLNNAKKT | <400>104 |
| YTLNNAKKTN | <400>105 | TLNNAKKTNV | <400>106 |
| TNNAKKTNVT | <400>107 | NNAKKTNVTL | <400>108 |
| NAKKTNVTLS | <400>109 | AKKTNVTLSK | <400>110 |
| KKTNVTLSKK | <400>111 | KTNVTLSKKR | <400>112 |
| TNVTLSKKRK | <400>113 | NVTLSKKRKR | <400>114 |
| VTLSKKRKRR | <400>115 | TLSKKRKRRF | <400>116 |
| LSKKRKRRFL | <400>117 | SKKRKRRFLG | <400>118 |
| KKRKRRFLGF | <400>119 | KRKRRFLGFL | <400>120 |
| RKRRFLGFLL | <400>121 | KRRFLGFLLG | <400>122 |
| RRFLGFLLGV | <400>123 | RFLGFLLGVG | <400>124 |
| FLGFLLGVGS | <400>125 | LGFLLGVGSA | <400>126 |
| GFLLGVGSAI | <400>127 | FLLGVGSAIA | <400>128 |
| LLGVGSAIAS | <400>129 | LGVGSAIASG | <400>130 |
| GVGSAIASGV | <400>131 | VGSAIASGVA | <400>132 |
| GSAIASGVAV | <400>133 | SAIASGVAVS | <400>134 |
| AIASGVAVSK | <400>135 | IASGVAVSKV | <400>136 |
| ASGVAVSKVL | <400>137 | SGVAVSKVLH | <400>138 |
| GVAVSKVLHL | <400>139 | VAVSKVLHLE | <400>140 |
| AVSKVLHLEG | <400>141 | VSKVLHLEGE | <400>142 |
| SKVLHLEGEV | <400>143 | KVLHLEGEVN | <400>144 |
| VLHLEGEVNK | <400>145 | LHLEGEVNKI | <400>146 |
| HLEGEVNKIK | <400>147 | LEGEVNKIKS | <400>148 |
| EGEVNKIKSA | <400>149 | GEVNKIKSAL | <400>150 |
| EVNKIKSALL | <400>151 | VNKIKSALLS | <400>152 |
| NKIKSALLST | <400>153 | KIKSALLSTN | <400>154 |
| IKSALLSTNK | <400>155 | KSALLSTNKA | <400>156 |
| SALLSTNKAV | <400>157 | ALLSTNKAVV | <400>158 |
| LLSTNKAVVS | <400>159 | LSTNKAVVSL | <400>160 |
| STNKAVVSLS | <400>161 | TNKAVVSLSN | <400>162 |
| NKAVVSLSNG | <400>163 | KAVVSLSNGV | <400>164 |
| AVVSLSNGVS | <400>165 | VVSLSNGVSV | <400>166 |
| VSLSNGVSVL | <400>167 | SLSNGVSVLT | <400>168 |
| LSNGVSVLTS | <400>169 | SNGVSVLTSK | <400>170 |
| NGVSVLTSKV | <400>171 | GVSVLTSKVL | <400>172 |
| VSVLTSKVLD | <400>173 | SVLTSKVLDL | <400>174 |
| VLTSKVLDLK | <400>175 | LTSKVLDLKN | <400>176 |
| TSKVLDLKNY | <400>177 | SKVLDLKNYI | <400>178 |
| KVLDLKNYID | <400>179 | VLDLKNYIDK | <400>180 |
| LDLKNYIDKQ | <400>181 | DLKNYIDKQL | <400>182 |
| LKNYIDKQLL | <400>183 | KNYIDKQLLP | <400>184 |
| NYIDKQLLPI | <400>185 | YIDKQLLPIV | <400>186 |
| IDKQLLPIVT | <400>187 | DKQLLPIVNK | <400>188 |
| KQLLPIVNKQ | <400>189 | QLLPIVNKQS | <400>190 |
| LLPIVNKQSC | <400>191 | LPIVNKQSCS | <400>192 |
| PIVNKQSCSI | <400>193 | IVNKQSCSIS | <400>194 |
| VNKQSCSISN | <400>195 | NKQSCSISNI | <400>196 |
| KQSCSISNIE | <400>197 | QSCSISNIET | <400>198 |
| SCSISNIETV | <400>199 | CSISNIETVI | <400>200 |
| SISNTETVIE | <400>201 | ISNIETVIEF | <400>202 |
| SNIETVIEFQ | <400>203 | NIETVIEFQQ | <400>204 |
| IETVIEFQQK | <400>205 | ETVIEFQQKN | <400>206 |
| TVIEFQQKNN | <400>207 | VIEFQQKNNR | <400>208 |
| IEFQQKNNRL | <400>209 | EFQQKNNRLL | <400>210 |
| FQQKNNRLLE | <400>211 | QQKNNRLLEI | <400>212 |
| QKNNRLLEIT | <400>213 | KNNRLLEITR | <400>214 |
| NNRLLEITRE | <400>215 | NRLLEITREF | <400>216 |
| RLLEITREFS | <400>217 | LLEITREFSV | <400>218 |
| LEITREFSVN | <400>219 | EITREFSVNA | <400>220 |
| ITREFSVNAG | <400>221 | TREFSVNAGV | <400>222 |
| REFSVNAGVT | <400>223 | EFSVNAGVTT | <400>224 |
| FSVNAGVTTP | <400>225 | SVNAGVTTPV | <400>226 |
| VNAGVTTPVS | <400>227 | NAGVTTPVST | <400>228 |
| AGVTTPVSTY | <400>229 | GVTTPVSTYM | <400>230 |
| VTTPVSTYML | <400>231 | TTPVSTYMLT | <400>232 |
| TPVSTYMLTN | <400>233 | PVSTYMLTNS | <400>234 |
| VSTYMLTNSE | <400>235 | STYMLTNSEL | <400>236 |
| TYMLTNSELL | <400>237 | YMLTNSELLS | <400>238 |
| MLTNSELLSL | <400>239 | LTNSELLSLI | <400>240 |
| TNSELLSLIN | <400>241 | NSELLSLIND | <400>242 |
| SELLSLINDM | <400>243 | ELLSLINDMP | <400>244 |
| LLSLINDMPI | <400>245 | LSLINDMPIT | <400>246 |
| SLINDMPITN | <400>247 | LINDMPITND | <400>248 |
| INDMPITNDQ | <400>249 | NDMPITNDQK | <400>250 |
| DMPITNDQKK | <400>251 | MPITNDQKKL | <400>252 |
| PITNDQKKLN | <400>253 | ITNDQKKLMS | <400>254 |
| TNDQKKLMSN | <400>255 | NDQKKLMSNN | <400>256 |
| DQKKLMSNNV | <400>257 | QKKLMSNNVQ | <400>258 |
| KKLMSNNVQI | <400>259 | KLMSNNVQIV | <400>260 |
| LMSNNVQIVR | <400>261 | MSNNVQIVRQ | <400>262 |
| SNNVQIVRQQ | <400>263 | NNVQIVRQQS | <400>264 |
| NVQIVRQQSY | <400>265 | VQIVRQQSYS | <400>266 |
| QIVRQQSYSI | <400>267 | IVRQQSYSIM | <400>268 |
| VRQQSYSIMS | <400>269 | RQQSYSIMSI | <400>270 |
| QQSYSIMSII | <400>271 | QSYSIMSIIK | <400>272 |
| SYSIMSIIKE | <400>273 | YSIMSIIKEE | <400>274 |
| SIMSIIKEEV | <400>275 | IMSIIKEEVL | <400>276 |
| MSIIKEEVLA | <400>277 | SIIKEEVLAY | <400>278 |
| IIKEEVLAYV | <400>279 | IKEEVLAYVV | <400>280 |
| KEEVLAYVVQ | <400>281 | EEVLAYVVQL | <400>282 |
| EVLAYVVQLP | <400>283 | VLAYVVQLPL | <400>284 |
| LAYVVQLPLY | <400>285 | AYVVQLPLYG | <400>286 |
| YVVQLPLYGV | <400>287 | VVQLPLYGVI | <400>288 |
| VQLPLYGVID | <400>289 | QLPLYGVIDT | <400>290 |
| LPLYGVIDTP | <400>291 | PLYGVIDTPC | <400>292 |
| LYGVIDTPCW | <400>293 | YGVIDTPCWK | <400>294 |
| GVIDTPCWKL | <400>295 | VIDTPCWKLH | <400>296 |
| IDTPCWKLHT | <400>297 | DTPCWKLHTS | <400>298 |
| TPCWKLHTSP | <400>299 | PCWKLHTSPL | <400>300 |
| CWKLHTSPLC | <400>301 | WKLHTSPLCT | <400>302 |
| KLHTSPLCTT | <400>303 | LHTSPLCTTN | <400>304 |
| HTSPLCTTNT | <400>305 | TSPLCTTNTK | <400>306 |
| SPLCTTNTKE | <400>307 | PLCTTNTKEG | <400>308 |
| LCTTNTKEGS | <400>309 | CTTNTKEGSN | <400>310 |
| TTNTKEGSNI | <400>311 | TNTKEGSNTC | <400>312 |
| NTKEGSNICL | <400>313 | TKEGSNICLT | <400>314 |
| KEGSNICLTR | <400>315 | EGSNICLTRT | <400>316 |
| GSNICLTRTD | <400>317 | SNICLTRTDR | <400>318 |
| NICLTRTDRG | <400>319 | ICLTRTDRGW | <400>320 |
| CLTRTDRGWY | <400>321 | LTRTDRGWYC | <400>322 |
| TRTDRGWYCD | <400>323 | RTDRGWYCDN | <400>324 |
| TDRGWYCDNA | <400>325 | DRGWYCDNAG | <400>326 |
| RGWYCDNAGS | <400>327 | GWYCDNAGSV | <400>328 |
| WYCDNAGSVS | <400>329 | YCDNAGSVSF | <400>330 |
| CDNAGSVSFF | <400>331 | DNAGSVSFFP | <400>332 |
| NAGSVSFFPQ | <400>333 | AGSVSFFPQA | <400>334 |
| GSVSFFPQAE | <400>335 | SVSFFPQAET | <400>336 |
| VSFFPQAETC | <400>337 | SFFPQAETCK | <400>338 |
| FFPQAETCKV | <400>339 | FPQAETCKVQ | <400>340 |
| PQAETCKVQS | <400>341 | QAETCKVQSN | <400>342 |
| AETCKVQSNR | <400>343 | ETCKVQSNRV | <400>344 |
| TCKVQSNRVF | <400>345 | CKVQSNRVFC | <400>346 |
| KVQSNRVFCD | <400>347 | VQSNRVFCDT | <400>348 |
| QSNRVFCDTM | <400>349 | SNRVFCDTMN | <400>350 |
| NRVFCDTMNS | <400>351 | RVFCDTMNSL | <400>352 |
| VFCDTMNSLT | <400>353 | FCDTMNSLTL | <400>354 |
| CDTMNSLTLP | <400>355 | DTMNSLTLPS | <400>356 |
| TMNSLTLPSE | <400>357 | MNSLTLPSEV | <400>358 |
| NSLTLPSEVN | <400>359 | SLTLPSEVNL | <400>360 |
| LTLPSEVNLC | <400>361 | TLPSEVNLCN | <400>362 |
| LPSEVNLCNV | <400>363 | PSEVNLCNVD | <400>364 |
| SEVNLCNVDI | <400>365 | EVNLCNVDIF | <400>366 |
| VNLCNVDIFN | <400>367 | NLCNVDIFNP | <400>368 |
| LCNVDIFNPK | <400>369 | CNVDIFNPKY | <400>370 |
| NVDIFNPKYD | <400>371 | VDIFNPKYDC | <400>372 |
| DIFNPKYDCK | <400>373 | IFNPKYDCKI | <400>374 |
| FNPKYDCKIM | <400>375 | NPKYDCKIMT | <400>376 |
| PKYDCKIMTS | <400>377 | KYDCKIMTSK | <400>378 |
| YDCKIMTSKT | <400>379 | DCKIMTSKTD | <400>380 |
| CKIMTSKTDV | <400>381 | KIMTSKTDVS | <400>382 |
| IMTSKTDVSS | <400>383 | MTSKTDVSSS | <400>384 |
| TSKTDVSSSV | <400>385 | SKTDVSSSVI | <400>386 |
| KTDVSSSVIT | <400>387 | TDVSSSVITS | <400>388 |
| DVSSSVITSL | <400>389 | VSSSVITSLG | <400>390 |
| SSSVITSLGA | <400>391 | SSVITSLGAI | <400>392 |
| SVITSLGAIV | <400>393 | VITSLGAIVS | <400>394 |
| ITSLGAIVSC | <400>395 | TSLGAIVSCY | <400>396 |
| SLGAIVSCYG | <400>397 | LGAIVSCYGK | <400>398 |
| GAIVSCYGKT | <400>399 | AIVSCYGKTK | <400>400 |
| IVSCYGKTKC | <400>401 | VSCYGKTKCT | <400>402 |
| SCYGKTKCTA | <400>403 | CYGKTKCTAS | <400>404 |
| YGKTKCTASN | <400>405 | GKTKCTASNK | <400>406 |
| KTKCTASNKN | <400>407 | TKCTASNKNR | <400>408 |
| KCTASNKNRG | <400>409 | CTASNKNRGI | <400>410 |
| TASNKNRGII | <400>411 | ASNKNRGIIK | <400>412 |
| SNKNRGIIKT | <400>413 | NKNRGIIKTF | <400>414 |
| KNRGIIKTFS | <400>415 | NRGITKTFSN | <400>416 |
| RGIIKTFSNG | <400>417 | GITKTFSNGC | <400>418 |
| IIKTFSNGCD | <400>419 | TKTFSNGCDY | <400>420 |
| KTFSNGCDYV | <400>421 | TFSNGCDYVS | <400>422 |
| FSNGCDYVSN | <400>423 | SNGCDYVSNK | <400>424 |
| NGCDYVSNKG | <400>425 | GCDYVSNKGV | <400>426 |
| CDYVSNKGVD | <400>427 | DYVSNKGVDT | <400>428 |
| YVSNKGVDTV | <400>429 | VSNKGVDTVS | <400>430 |
| SNKGVDTVSV | <400>431 | NKGVDTVSVG | <400>432 |
| KGVDTVSVGN | <400>433 | GVDTVSVGNT | <400>434 |
| VDTVSVGNTL | <400>435 | DTVSVGNTLY | <400>436 |
| TVSVGNTLYY | <400>437 | VSVGNTLYYV | <400>438 |
| SVGNTLYYVN | <400>439 | VGNTLYYVNK | <400>440 |
| GNTLYYVNKQ | <400>441 | NTLYYVNKQE | <400>442 |
| TLYYVNKQEG | <400>443 | LYYVNKQEGK | <400>444 |
| YYVNKQEGKS | <400>445 | YVNKQEGKSL | <400>446 |
| VNKQEGKSLY | <400>447 | NKQEGKSLYV | <400>448 |
| KQEGKSLYVK | <400>449 | QEGKSLYVKG | <400>450 |
| EGKSLYVKGE | <400>451 | GKSLYVKGEP | <400>452 |
| KSLYVKGEPI | <400>453 | SLYVKGEPII | <400>454 |
| LYVKGEPIIN | <400>455 | YVKGEPIINF | <400>456 |
| VKGEPIINFY | <400>457 | KGEPIINFYD | <400>458 |
| GEPIINFYDP | <400>459 | EPIINFYDPL | <400>460 |
| PIINFYDPLV | <400>461 | IINFYDPLVF | <400>462 |
| INFYDPLVFP | <400>463 | NFYDPLVFPS | <400>464 |
| FYDPLVFPSD | <400>465 | YDPLVFPSDE | <400>466 |
| DPLVFPSDEF | <400>467 | PLVFPSDEFD | <400>468 |
| LVFPSDEFDA | <400>469 | VFPSDEFDAS | <400>470 |
| FPSDEFDASI | <400>471 | PSDEFDASIS | <400>472 |
| SDEFDASISQ | <400>473 | DEFDASISQV | <400>474 |
| EFDASISQVN | <400>475 | FDASISQVNE | <400>476 |
| DASISQVNEK | <400>477 | ASISQVNEKI | <400>478 |
| SISQVNEKIN | <400>479 | ISQVNEKINQ | <400>480 |
| SQVNEKINQS | <400>481 | QVNEKINQSL | <400>482 |
| VNEKINQSLA | <400>483 | NEKINQSLAF | <400>484 |
| EKINQSLAFI | <400>485 | KINQSLAFIR | <400>486 |
| INQSLAFIRK | <400>487 | NQSLAFIRKS | <400>488 |
| QSLAFIRKSD | <400>489 | SLAFIRKSDE | <400>490 |
| LAFIRKSDEL | <400>491 | AFIRKSDELL | <400>492 |
| FIRKSDELLH | <400>493 | IRKSDELLHN | <400>494 |
| RKSDELLHNV | <400>495 | KSDELLHNVN | <400>496 |
| SDELLHNVNA | <400>497 | DELLHNVNAG | <400>498 |
| ELLHNVNAGK | <400>499 | LLHNVNAGKS | <400>500 |
| LKNVNAGKST | <400>501 | HNVNAGKSTT | <400>502 |
| NVNAGKSTTN | <400>503 | VNAGKSTTNI | <400>504 |
| NAGKSTTNIM | <400>505 | AGKSTTNIMI | <400>506 |
| GKSTTNIMIT | <400>507 | KSTTNIMITT | <400>508 |
| STTNIMITTI | <400>509 | TTNIMITTII | <400>510 |
| TNIMITTIII | <400>511 | NIMITTIIIV | <400>512 |
| IMITTIIIVI | <400>513 | MITTIIIVII | <400>514 |
| ITTIIIVIIV | <400>515 | TTIIIVIIVI | <400>516 |
| TIIIVIIVIL | <400>517 | IIIVIIVILL | <400>518 |
| IIVIIVILLS | <400>519 | IVIIVILLSL | <400>520 |
| VIIVILLSLI | <400>521 | IIVILLSLIA | <400>522 |
| IVILLSLIAV | <400>523 | VILLSLIAVG | <400>524 |
| ILLSLIAVGL | <400>525 | LLSLIAVGLL | <400>526 |
| LSLIAVGLLL | <400>527 | SLIAVGLLLY | <400>528 |
| LIAVGLLLYC | <400>529 | IAVGLLLYCK | <400>530 |
| AVGLLLYCKA | <400>531 | VGLLLYCKAR | <400>532 |
| GLLLYCKARS | <400>533 | LLLYCKARST | <400>534 |
| LLYCKARSTP | <400>535 | LYCKARSTPV | <400>536 |
| YCKARSTPVT | <400>537 | CKARSTPVTL | <400>538 |
| KARSTPVTLS | <400>539 | ARSTPVTLSK | <400>540 |
| RSTPVTLSKD | <400>541 | STPVTLSKDQ | <400>542 |
| TPVTLSKDQL | <400>543 | PVTLSKDQLS | <400>544 |
| VTLSKDQLSG | <400>545 | TLSKDQLSGI | <400>546 |
| LSKDQLSGIN | <400>547 | SKDQLSGINN | <400>548 |
| KDQLSGINNI | <400>549 | DQLSGINNIA | <400>550 |
| QLSGINNIAF | <400>551 | LSGINNIAFS | <400>552 |
| SGINNIAFSN | <400>553 | | |

Even more preferably said antagonist interacts with a sequence selected from <400>88, <400>89, <400>90, <400>91, <400>92, <400>93 or <400>94.

Reference to “interacts” should be understood as a reference to any form of interaction including, but not limited to covalent bonds, hydrogen bonds, ionic bonds, van der Waals forces or any other interactive bonding mechanism.

Still without limiting the present invention to any one theory or mode of action the inventors have determined that inhibition or other form of interference with cleavage at the newly identified cleavage site disclosed herein interferes with F protein functioning. Further, it is thought that the intervening sequence exhibits relevance in relation to immune recognition. Specifically, it is thought that F proteins engineered to either retain the intervening sequence or which are engineered such that the intervening sequence is removed altogether exhibit altered but improved immunogenicity. Although not wishing to be constrained by theory, it is thought that in the normal physiological setting, the intervening sequence which is excised following formation of the fully functional F glycoprotein serves as an immune decoy thereby obstructing or otherwise inhibiting the induction of an immune response against the fully functional F protein.

Accordingly, mutating the cleavage sites comprising the F protein (at either the amino acid or encoding nucleic acid level) provides a useful tool for producing molecules which are engineered to retain the intervening sequence and which cannot undergo the normal cleavage event in order to generate the fully functional F protein. These molecules are useful in a range of applications including, but not limited to, as an immunogen for use in a vaccination protocol. In addition to producing a F protein variant which cannot be cleaved, identification by the inventors of the second cleavage site now enables the synthesis of F protein molecules which lack the intervening sequence as herein defined. This is particularly useful since it is thought that the F protein which lacks the intervening sequence, but which intervening sequence was not released into the circulation of the subject, will exhibit better immunogenecity than the naturally occurring F protein.

Accordingly, in another aspect there is provided a viral F protein variant comprising a mutation in the intervening peptide sequence wherein said variant exhibits modulated functional activity relative to wild type F protein or a derivative, homologue, analogue, chemical equivalent or mimetic of said variant.

More particularly, there is provided a viral F protein variant comprising a mutation in the intervening peptide sequence wherein said variant exhibits down-regulated functional activity relative to wild type F protein or a derivative, homologue, analogue, chemical equivalent or mimetic of said variant.

Reference to “intervening peptide sequence” should be understood to have the same meaning as hereinbefore defined.

Reference to “wild type” F protein is a reference to the forms of F protein which are predominantly expressed by negative sense single stranded RNA viruses. This should be understood to include reference to the uncleaved form of the F protein, the functional activity of which includes the capacity to undergo cleavage and excision of the intervening sequence, and the fully functional F protein in respect of which the intervening sequence has been excised. It should be understood that to the extent that the subject variant molecule comprises all or part of the intervening sequence, modulation of its functional activity should be assessed relative to the wild type F protein which still comprises the intervening sequence. Conversely, a variant F protein which does not comprise the intervening sequence should be assessed relative to the cleaved wild type F protein. In this regard, reference to “functional activity” should be understood as a reference to any one or more of the functional activities which the subject F protein can perform including, but not limited to, its capacity to undergo cleavage or its capacity to induce an immune response.

Reference to “mutation” should be understood as a reference to any change, alteration or other modification, whether occurring naturally or non-naturally, which results in the subject F protein exhibiting functional activity which is modulated relative to that of the corresponding wild type F protein.

The change, alteration or other modification may take any form including, but not limited to, a structural modification (such as an alteration secondary, tertiary or quaternary structure of the F protein molecule), a molecular modification (such as an addition substitutional deletion of one or more amino acids from the F protein) or a chemical modification. The subject modification should also be understood to extend to the fusion, linking or binding of a proteinaceous or non-proteinaceous molecule to the F protein or to the nucleic acid molecule encoding the F protein thereby rendering the expression product functionally distinctive over the corresponding wild type F protein. It should also be understood that although it is necessary that the subject mutation is expressed by the F protein expression product, the creation of the mutation may be achieved by any suitable means including either mutating a wild type F protein, synthesising a F protein variant or modifying a nucleic acid molecule encoding a wild type F protein such that the expression product of said mutated nucleic acid molecule is a F protein variant. Preferably, said mutation is a single or multiple amino acid sequence substitution, addition and/or deletion. In this regard, in one preferred embodiment the subject mutation is deletion of all or part of the intervening sequence. In another preferred embodiment, the subject mutation is an amino acid substitution which renders the newly identified cleavage site inactive. By inactive is meant that the cleavage site cannot be cleaved by the enzymatic processes which normally function to activate an F protein in vivo.

In a preferred embodiment the viral F protein is a Paramyxoviridae F protein and still more preferably the subject viral F protein is of the Genus Pneumovirus and still more preferably respiratory syncytial virus.

In a most preferred embodiment there is provided a respiratory syncytial virus F protein variant comprising a mutation in the cleavage site defined by amino acids RARR (<400>564) wherein said variant exhibits down-regulated functional activity relative to wild type F protein or a derivative, homologue, analogue, chemical equivalent or mimetic of said variant.

Preferably said mutation comprises one or more of the amino acid substitutions selected from the following list:

(i) R106G

(ii) A107Q

(iii) R108G

Still more preferably said F protein variant comprises the sequence substantially as set forth in<400>565.

In another preferred embodiment there is provided a respiratory syncytial virus F protein variant comprising a multiple amino acid deletion from the intervening peptide sequence wherein said variant exhibits down-regulated functional activity relative to wild type F protein or a derivative, homologue, analogue, chemical equivalent of said variant.

It is more preferably provided that said amino acid deletion is a partial deletion of the intervening peptide sequence and more preferably a deletion of the peptide sequence

RARRELPRFMNYTLNNAKKTNVTLS <400>569.

Still more preferably said variant comprises the amino acid sequence substantially as set forth in<400>567.

To the extent that the present invention relates to F protein variants comprising one or more amino acid additions, substitutions and/or deletions, it should also be understood to extend to nucleic acid molecules encoding said variants.

Accordingly, another aspect of the present invention is directed to an isolated nucleic acid molecule selected from the list consisting of:

(i) An isolated nucleic acid molecule or derivative or equivalent thereof comprising a nucleotide sequence encoding or complementary to a sequence encoding a viral F protein variant or derivative, homologue, analogue, chemical equivalent or mimetic of said variant, which variant comprises a mutation in the intervening peptide sequence wherein said variant exhibits modulated functional activity relative to wild-type F protein.

(ii) An isolated nucleic acid molecule or derivative or equivalent thereof comprising a nucleotide sequence encoding or complementary to a sequence encoding a viral F protein variant or derivative, homologue, analogue, chemical equivalent or mimetic of said variant, which variant comprises a mutation in the intervening peptide sequence wherein said variant exhibits down-regulated functional activity relative to wild-type F protein.

(iii) An isolated nucleic acid molecule or derivative or equivalent thereof comprising a nucleotide sequence encoding or complementary to a sequence encoding a respiratory syncytial virus F protein or derivative, homologue, analogue, chemical equivalent or mimetic of said variant, which variant comprises a mutation in the cleavage site defined by amino acids RARR wherein said variant exhibits down-regulated functional activity relative to wild-type F protein.

(iv) An isolated nucleic acid molecule or derivative or equivalent thereof comprising a nucleotide sequence encoding or complementary to a sequence encoding a respiratory syncytial virus F protein variant or derivative, homologue, analogue, chemical equivalent or mimetic of said variant, which variant comprises one or more of the amino acid substitutions selected from the following list:

(a) R106G

(b) A107Q

(c) R108G

(v) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises a multiple amino acid deletion from the intervening peptide sequence wherein said variant exhibits down-regulated functional activity relative to wild-type F protein.

(vi) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises a partial deletion of the intervening peptide sequence and more preferably a deletion of the peptide sequence

RARRELPRFMNYTLNNAKKTNVTLS <400>569.

(vii) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises the amino acid sequence substantially as set forth in<400>567.

(viii) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises the amino acid sequence substantially as set forth in<400>565.

(ix) An isolated nucleic acid molecule or derivative or analogue thereof comprising the nucleotide substantially as set forth in<400>568.

(x) An isolated nucleic acid molecule or derivative or analogue thereof comprising the nucleotide substantially as set forth in<400>566.

In a preferred embodiment the viral F protein is a Paramyxoviridae F protein and still more preferably the subject viral F protein is of the Genus Pneumovirus and still more preferably respiratory syncytial virus.

The nucleic acid molecule of the subject invention may be ligated to an expression vector capable of expression in a prokaryotic cell (eg. E. Coli) or a eukaryotic cell (eg. yeast cells, fungal cells, insect cells, mammalian cells or plant cells). The nucleic acid molecule may be ligated or fused or otherwise associated with a nucleic acid molecule encoding another entity such as, for example, a signal peptide. It may also comprise additional nucleotide sequence information fused, linked or otherwise associated with it either at the 3′ or 5′ terminal portions or at both the 3′ and 5′ terminal portions. The nucleic acid molecule may also be part of a vector, such as an expression vector. The latter embodiment facilitates production of recombinant forms of the variant F protein encompassed by the present invention.

The variant F protein molecule of the present invention may be derived from natural or recombinant sources or may be chemically synthesised. Methods for producing these molecules would be well known to those skilled in the art.

As hereinbefore provided, “derivatives” include fragments, parts, portions, variants and mimetics from natural, synthetic or recombinant sources including fusion proteins. Parts or fragments include, for example, active regions of F protein. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the protein although random insertion is also possible with suitable screening of the resulting product. Deletional variants are characterised by the removal of one or more amino acids from the sequence. Substitutional amino acid variants are those in which at least one residue in the sequence has been removed and a different residue inserted in its place. An example of substitutional amino acid variants are conservative amino acid substitutions.

Conservative amino acid substitutions typically include substitutions within the following groups: glycine and alanine; valine, isoleucine and leucine; aspartic acid and glutamic acid; asparagine and glutamine; serine and threonine; lysine and arginine; and phenylalanine and tyrosine. Additions to amino acid sequences include fusions with other peptides, polypeptides or proteins.

Reference to “homologues” should be understood as a reference to F protein nucleic acid molecules or proteins derived from viral strains other than the species of origin.

Chemical and functional equivalents of F protein nucleic acid or protein molecules should be understood as molecules exhibiting any one or more of the functional activities of these molecules and may be derived from any source such as being chemically synthesized or identified via screening processes such as natural product screening.

The derivatives include fragments having particular epitopes or parts of the entire protein fused to peptides, polypeptides or other proteinaceous or non-proteinaceous molecules.

Analogues contemplated herein include, but are not limited to, modification to side chains, incorporating of unnatural amino acids and/or their derivatives during peptide, polypeptide or protein synthesis and the use of crosslinkers and other methods which impose conformational constraints on the proteinaceous molecules or their analogues.

Derivatives of nucleic acid sequences may similarly be derived from single or multiple nucleotide substitutions, deletions and/or additions including fusion with other nucleic acid molecules. The derivatives of the nucleic acid molecules of the present invention include oligonucleotides, PCR primers, antisense molecules, molecules suitable for use in cosuppression and fusion of nucleic acid molecules. Derivatives of nucleic acid sequences also include degenerate variants.

Examples of side chain modifications contemplated by the present invention include modifications of amino groups such as by reductive alkylation by reaction with an aldehyde followed by reduction with NaBH₄; amidination with methylacetimidate; acylation with acetic anhydride; carbamoylation of amino groups with cyanate; trinitrobenzylation of amino groups with 2, 4, 6-trinitrobenzene sulphonic acid (TNBS); acylation of amino groups with succinic anhydride and tetrahydrophthalic anhydride; and pyridoxylation of lysine with pyridoxal-5-phosphate followed by reduction with NaBH₄.

The guanidine group of arginine residues may be modified by the formation of heterocyclic condensation products with reagents such as 2,3-butanedione, phenylglyoxal and glyoxal.

The carboxyl group may be modified by carbodiimide activation via O-acylisourea formation followed by subsequent derivitisation, for example, to a corresponding amide.

Sulphydryl groups may be modified by methods such as carboxymethylation with iodoacetic acid or iodoacetamide; performic acid oxidation to cysteic acid; formation of a mixed disulphides with other thiol compounds; reaction with maleimide, maleic anhydride or other substituted maleimide; formation of mercurial derivatives using 4-chloromercuribenzoate, 4-chloromercuriphenylsulphonic acid, phenylmercury chloride, 2-chloromercuri-4-nitrophenol and other mercurials; carbamoylation with cyanate at alkaline pH.

Tryptophan residues may be modified by, for example, oxidation with N-bromosuccinimide or alkylation of the indole ring with 2-hydroxy-5-nitrobenzyl bromide or sulphenyl halides. Tyrosine residues on the other hand, may be altered by nitration with tetranitromethane to form a 3-nitrotyrosine derivative.

Modification of the imidazole ring of a histidine residue may be accomplished by alkylation with iodoacetic acid derivatives or N-carboethoxylation with diethylpyrocarbonate.

Examples of incorporating unnatural amino acids and derivatives during protein synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids. A list of unnatural amino acids contemplated herein is shown in Table 3.

TABLE 3

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | Chexa |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | Dmorn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| γ-aminobutyric acid | Gabu |
| L-t-butylglycine | Tbug |
| L-ethylglycine | Etg |
| L-homophenylalanine | Hphe |
| L-α-methylarginine | Marg |
| L-α-methylaspartate | Masp |
| L-α-methylcysteine | Mcys |
| L-α-methylglutamine | Mgln |
| L-α-methylhistidine | Mhis |
| L-α-methylisoleucine | Mile |
| L-α-methylleucine | Mleu |
| L-α-methylmethionine | Mmet |
| L-α-methylnorvaline | Mnva |
| L-α-methylphenylalanine | Mphe |
| L-α-methylserine | Mser |
| L-α-methyltryptophan | Mtrp |
| L-α-methylvaline | Mval |
| N-(N-(2,2-diphenylethyl) | Nnbhm |
| carbamylmethyl)glycine | |
| 1-carboxy-1-(2,2-diphenyl-Nmbc | |
| ethylamino)cyclopropane | |
| L-N-methylalanine | Nmala |
| L-N-methylarginine | Nmarg |
| L-N-methylasparagine | Nmasn |
| L-N-methylaspartic acid | Nmasp |
| L-N-methylcysteine | Nmcys |
| L-N-methylglutamine | Nmgln |
| L-N-methylglutamic acid | Nmglu |
| L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl--aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-γ-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

Crosslinkers can be used, for example, to stabilise 3D conformations, using homo-bifunctional crosslinkers such as the bifunctional imido esters having (CH₂)ₙ spacer groups with n=1 to n=6, glutaraldehyde, N-hydroxysuccinimide esters and hetero-bifunctional reagents which usually contain an amino-reactive moiety such as N-hydroxysuccinimide and another group specific-reactive moiety.

In addition to screening for agents which modulate F protein functional activity, the development of a method of producing a viral F protein or derivative thereof in a eukaryotic cell and identification of the novel F protein cleavage site has now facilitated the development of in vivo methodology directed to administering to a subject a vaccine comprising a nucleic acid molecule encoding a viral F protein or derivative thereof. Reference to “derivative” should be understood to encompass variants thereof, such as the variants hereinbefore defined. Without limiting the present invention to any one theory or mode of action, the operation of such a vaccine is based on the generation of an immune response, in particular antibody synthesis, directed to the subject F protein or derivative thereof. The antibodies generated therein bind to virally produced F proteins thereby inhibiting their fusion related functional activity and consequently reducing and/or inhibiting further viral propagation. Such a vaccine is useful in either the prophylactic and/or therapeutic sense.

Accordingly, another aspect of the present invention provides a recombinant viral construct comprising a nucleic acid molecule encoding a viral F protein or derivative thereof, the nucleotide sequence of which nucleic acid molecule comprises codons optimised for expression in a eukaryotic cell, wherein said recombinant viral construct is effective in inducing, enhancing or otherwise stimulating an immune response to said F protein.

Still another aspect of the present invention provides a recombinant viral construct comprising a nucleic acid molecule encoding a viral F protein variant or derivative thereof wherein said recombinant viral construct is effective in inducing, enhancing or otherwise stimulating an immune response to said F protein variant.

In a preferred embodiment the viral F protein is a Paramyxoviridae F protein and still more preferably the subject viral F protein is of the Genus Pneumovirus and still more preferably respiratory syncytial virus.

Reference to “inducing, enhancing or otherwise stimulating” an immune response to an F protein should be understood to mean stimulating or facilitating the stimulation of a specific immune response. The specific immune response is preferably a humoral response which is directed to any one or more regions of the F protein. In this regard, it should be understood that the subject immune response will down-regulate and/or inhibit at least one functional activity of the subject F protein.

Yet another aspect of the present invention relates to a vaccine comprising a recombinant viral construct which construct comprises a nucleic acid molecule encoding a respiratory syncytial virus F protein or derivative thereof, the nucleotide sequence of which nucleic acid molecule is optimised for expression in a eukaryotic cell wherein said recombinant viral construct is effective in inducing, enhancing or otherwise stimulating an immune response to said F protein.

Still another aspect of the present invention relates to a vaccine comprising a recombinant viral construct which construct comprises a nucleic acid molecule encoding a respiratory syncytial virus F protein variant or derivative thereof, wherein said recombinant viral construct is effective in inducing, enhancing or otherwise stimulating an immune response to said F protein variant.

In accordance with these aspects of the present invention, the nucleotide sequence of the subject nucleic acid molecule is preferably the nucleotide sequence defined in <400>5, <400>6, <400>566 or <400>568.

A further aspect of the present invention relates to use of the agents hereinbefore defined to modulate F protein functional activity and, in particular, the use of these agents in the therapeutic and/or prophylactic treatment of conditions characterised by infection with a negative sense single stranded RNA virus, and more particularly respiratory syncytial virus. Conditions envisaged herein include Parainfluenza induced croup and bronchiolitis. It should be understood that reference to “agent” hereinafter includes reference to agents identified or generated by the screening assays described above, including the modulatory agents (for example, antibodies) which are generated in vivo via use of a DNA vaccine. This aspect of the present invention is also directed to use of the F protein or derivatives thereof or encoding nucleic acid molecules, including the F protein variants, as hereinbefore described in the therapeutic and/or prophylactic treatment of conditions characterised by infection with a negative sense single stranded RNA virus.

Accordingly, another aspect of the present invention provides the method of modulating at least one functional activity associated with a viral F protein in a subject, said method comprising introducing into said subject and effective amount of an F protein modulatory agent for a time and under condition sufficient for said agent to interact with said F protein.

Preferably, said functional activity is F protein mediated host cell-virion fusion and/or virion budding and said modulation is down-regulation.

In a preferred embodiment the viral F protein is a Paramyxoviridae F protein and still more preferably the subject viral F protein is of the Genus Pneumovirus and still more preferably respiratory syncytial virus.

The term “subject” includes humans primates, livestock animals (eg, horses, cattle, sheep, pigs, donkeys), laboratory test animals (eg, mice, rats, rabbits, guinea pigs), companion animals (eg, dogs, cats), captive wild animals (eg, kangaroos, deer, foxes), birds (eg, chickens, ducks, bantams, pheasants). Preferably the subject is a human or laboratory test animal. Even more preferably the subject is a human.

In another aspect, the present invention provides a method of modulating at least one functional activity associated with a viral F protein, said method comprising contacting said viral F protein with an effective amount of an F protein modulatory agent for a time and under conditions sufficient for said agent to interact with said F protein.

Preferably said viral F protein is a Pneumovirus F protein and even more preferably a respiratory syncytial virus F protein. Still more preferably said modulation is down-regulation of F protein functional activity.

This aspect of the present invention should be understood to extend to the modulation of F protein associated functional activities in in vitro culture systems. This may be of benefit, for example, when applied to in vitro procedures designed to virally infect a prospective host cell. This may be of particular use, for example, where it is desired to create a cell line or to otherwise create a virally transformed cell. In this regard, the subject modulation would preferably be up-regulation of F protein functional activity.

In yet another aspect, the present invention relates to a method for the treatment and/or prophylaxis of a condition characterised by infection with a negative sense single stranded RNA virus in a subject, said method comprising administering to said subject an effective amount of an agent, which agent is capable of down-regulating at least one functional activity of the F protein expressed by said virus, for a time and under conditions sufficient for said agent to interact with said F protein.

In still yet another aspect, the present invention relates to a method for the treatment and/or prophylaxis of a condition characterised by infection with a negative sense single stranded RNA virus in a subject, said method comprising administering to said subject an effective amount of a composition comprising an F protein or derivative thereof, F protein variant or derivative thereof and/or a nucleic acid molecule encoding said F protein or F protein variant as hereinbefore defined or a derivative, homologue, analogue, chemical equivalent a mimetic of said protein or nucleic acid molecule for a time and under conditions sufficient for said composition to down-regulate said viral F protein functional activity.

In a preferred embodiment the viral F protein is a Paramyxoviridae F protein and still more preferably the subject viral F protein is of the Genus Pneumovirus and still more preferably respiratory syncytial virus.

Reference to “administering” an agent should be understood to extend to the administration of a DNA vaccine for the purpose of in vivo generation of anti-F protein antibodies.

Reference to a condition “characterised by infection with a negative sense single stranded RNA virus” should be understood as a reference to a condition, one or more symptoms of which are directly or indirectly induced due to infection of the subject with the subject virus. Preferably, said virus is a Pneumovirus and even more preferably respiratory syncytial virus.

The molecule which may be administered to a subject in accordance with the present invention may also be linked to a targeting means such as a monoclonal antibody, which provides specific delivery of the molecule to the target cells.

In a preferred embodiment the subject of the prophylactic or therapeutic treatment is a mammal and still more preferably a human.

Administration of the subject modulatory agent or the subject F protein or derivative thereof, F protein variant or derivative thereof, nucleic acid molecule encoding said F protein or F protein variant as hereinbefore defined or a derivative, homologue, analogue, chemical equivalent or mimetic of said protein or nucleic acid molecule (hereinafter said modulatory agents, proteins and/or nucleic acid molecules are collectively referred to as the “active ingredients”), in the form of a pharmaceutical composition, may be performed by any convenient means. The active ingredients of the pharmaceutical composition are contemplated to exhibit therapeutic activity when administered in an amount which depends on the particular case. The variation depends, for example, on the human or animal and the active ingredient chosen. A broad range of doses may be applicable. Considering a patient, for example, from about 0.1 mg to about 1 mg of active ingredient may be administered per kilogram of body weight per day. Dosage regimes may be adjusted to provide the optimum therapeutic response. For example, several divided doses may be administered daily, weekly, monthly or other suitable time intervals or the dose may be proportionally reduced as indicated by the exigencies of the situation. The active ingredient may be administered in the form of pharmaceutically acceptable nontoxic salts, such as acid addition salts or metal complexes, e.g. with zinc, iron or the like (which are considered as salts for purposes of this application). Illustrative of such acid addition salts are hydrochloride, hydrobromide, sulphate, phosphate, maleate, acetate, citrate, benzoate, succinate, malate, ascorbate, tartrate and the like. If the active ingredient is to be administered in tablet form, the tablet may contain a binder such as tragacanth, corn starch or gelatin; a disintegrating agent, such as alginic acid; and a lubricant, such as magnesium stearate.

Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, infusion, orally, rectally, via IV drip patch and implant. Preferably, the route of administration is a route which permits directed delivery of the modulatory agent. For example, aerosol administration (such as by nebulisation) into the airways permits directed delivery to the airways region, in contrast to systemic delivery which results in delivery to the whole body.

Where the disorder which is the subject of treatment or prophylaxis is a respiratory distress syndrome, delivery of the active ingredient to the airway, for example as an aerosol via nebulisation, is an ideal approach since this maximises delivery to the airway where the infection has occurred and minimises systemic delivery which may be associated with side effects.

The term “aerosol” is used in its most general sense to include any formulation capable of administration via nasal, pharyngeal, tracheal, bronchial or oral passages. Aerosols generally comprise particles of liquid or solid suspended in a gas or vapour. Conveniently, the aerosol is a colloidal system such as a mist in which the dispersion medium is a gas. The method of administering the aerosol formulation is not critical and may be achieved using a nasal spray hand pump, electric pump, pressurised dispenser, nasal drip or other convenient means. Alternatively, the formulation may be administered in a dry powder delivery system. It should be understood that the method of the present invention extends to direct application of said formulations to intra nasal surfaces. In a particularly preferred embodiment, the aerosol is delivered at a rate of from about 1 to about 20 litres/min. and preferably from about 2 to about 15 litres/min. at a droplet size of from about 0.1 to about 10 μm and more preferably from about 0.1 to about 6 μm. Conveniently, a stock solution of material is prepared at a concentration of from about 0.5 to about 20 mg/ml or more preferably from about 1.0 to about 10 mg/ml of carrier solution.

The formulation is administered in a therapeutically effective amount. A therapeutically effective amount means that amount necessary at least partly to attain the desired effect, or to delay the onset of, inhibit the progression of, or halt altogether, the onset or progression of the particular condition being treated. Such amounts will depend, of course, on the particular conditions being treated, the severity of the condition and individual patient parameters including age, physical conditions, size, weight and concurrent treatment. These factors are well known to those of ordinary skill in the art and can be addressed with no more than routine experimentation. It is preferred generally that a maximum dose be used, that is, the highest safe dose according to sound medical judgement. It will be understood by those of ordinary skill in the art, however, that a lower dose or tolerable dose may be administered for medical reasons, psychological reasons or for virtually any other reasons.

Generally, daily doses of formulation will be from about 0.01 μg/kg per day to 1000 mg/kg per day. Small doses (0.01-1 mg) may be administered initially, followed by increasing doses up to about 1000 mg/kg per day. In the event that the response in a subject is insufficient at such doses, even higher doses (or effective higher doses by a different, more localised delivery route) may be employed to the extent patient tolerance permits. A single dose may be administered or multiple doses may be required on an hourly, daily, weekly or monthly basis. Effective amounts of formulation vary depending on the individual but may range from about 0.1 μg to about 20 mg, alternatively from about 1 μg to about 10 mg and more preferably from about 1 μg to 5 mg per dose.

In another aspect the present invention relates to the use of an agent capable of modulating at least one functional activity of a viral F protein, which agent is identified and/or generated in accordance with the methods hereinbefore defined, in the manufacture of a medicament for the treatment and/or prophylaxis of a condition characterised by infection with a negative sense single stranded RNA virus.

In still another aspect the present invention relates to the use of a composition comprising an F protein or derivative thereof, F protein variant or derivative thereof, nucleic acid molecule encoding said F protein or F protein variant as hereinbefore defined or a derivative, homologue, analogue, chemical equivalent or mimetic of said protein or nucleic acid molecule, in the manufacture of a medicament for the treatment and/or prophylaxis of a condition characterised by infection with a negative sense single stranded RNA virus.

In a preferred embodiment the viral F protein is a Paramyxoviridae F protein and still more preferably the subject viral F protein is of the Genus Pneumovirus and still more preferably respiratory syncytial virus.

In another aspect the present invention relates to the use of an agent, which agent is identified in accordance with the methods hereinbefore defined, in the manufacture of a medicament for the modulation of at least one viral F protein associated functional activity.

In a preferred embodiment the viral F protein is a Paramyxoviiidae F protein and still more preferably the subject viral F protein is of the Genus Pneumovirus and still more preferably respiratory syncytial virus.

Yet another aspect relates to agents for use in modulating the functional activity of a viral F protein wherein said agent is identified in accordance with the methods hereinbefore defined.

Still yet another aspect relates to agents for use in the treatment and/or prophylaxis of a condition characterised by infection with a negative sense single stranded RNA virus wherein said agent is identified in accordance with the methods hereinbefore defined.

Yet still another aspect relates to a composition comprising an F protein or derivative thereof, F protein variant or derivative thereof, a nucleic acid molecule encoding said F protein or F protein variant as hereinbefore defined or a derivative, homologue, analogue, chemical equivalent or mimetic of said protein or nucleic acid molecule for use in the treatment and/or prophylaxis of a condition characterised by infection with a negative sense single stranded RNA virus.

In a preferred embodiment the viral F protein is a Paramyxoviridae F protein and still more preferably the subject viral F protein is of the Genus Pneumovirus and still more preferably respiratory syncytial virus.

Reference herein to “treatment” and “prophylaxis” is to be considered in its broadest context. The term “treatment” does not necessarily imply that a mammal is treated until total recovery. Similarly, “prophylaxis” does not necessarily mean that the subject will not eventually contract a disease condition. Accordingly, treatment and prophylaxis include amelioration of the symptoms of a particular condition or preventing or otherwise reducing the risk of developing a particular condition. The term “prophylaxis” may be considered as reducing the severity of onset of a particular condition. “Treatment” may also reduce the severity of an existing condition or the frequency of acute attacks.

In accordance with these methods, the active ingredients defined in accordance with the present invention may be coadministered with one or more other compounds or molecules. By “coadministered” is meant simultaneous administration in the same formulation or in two different formulations via the same or different routes or sequential administration by the same or different routes. By “sequential” administration is meant a time difference of from seconds, minutes, hours or days between the administration of the two types of molecules. These molecules may be administered in any order.

In yet another aspect the present invention relates to a pharmaceutical composition comprising an active ingredient, as hereinbefore defined, and one or more pharmaceutically acceptable carriers and/or diluents.

The pharmaceutical forms suitable for injectable use include sterile aqueous solutions (where water soluble) or dispersions and sterile powders for the extemporaneous preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof and vegetable oils. The proper fluidity can be maintained, for example, by the use of a coating such as lecithin, by the maintenance of the required particle size in the case of dispersion and by the use of superfactants. The preventions of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions are prepared by incorporating the active compounds in the required amount in the appropriate solvent with various of the other ingredients enumerated above, as required, followed by filtered sterilisation. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

When the active ingredients are suitably protected they may be orally administered, for example, with an inert diluent or with an assimilable edible carrier, or it may be enclosed in hard or soft shell gelatin capsule, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. Such compositions and preparations should contain at least 1% by weight of active compound. The percentage of the compositions and preparations may, of course, be varied and may conveniently be between about 5 to about 80% of the weight of the unit. The amount of active compound in such therapeutically useful compositions in such that a suitable dosage will be obtained. Preferred compositions or preparations according to the present invention are prepared so that an oral dosage unit form contains between about 0.1 μg and 2000 mg of active compound.

The tablets, troches, pills, capsules and the like may also contain the components as listed hereafter: a binder such as gum, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound(s) may be incorporated into sustained-release preparations and formulations.

Pharmaceutical compositions suitable for aerosol administration have been hereinbefore described.

The pharmaceutical composition may also comprise genetic molecules such as a vector capable of transfecting target cells where the vector carries a nucleic acid molecule encoding an active ingredient. The vector may, for example, be a viral vector.

The present invention is further described by the following non-limiting examples.

TABLE 1

| Sequence ID | |
|---|---|
| Number | Description |
| <400>1 | Natural F protein nucleic acid sequence |
| <400>2 | Natural Fₛₒₗ portion nucleic acid sequence |
| <400>3 | Restriction site modified F protein nucleic |
| | acid sequence |
| <400>4 | Restriction site modified Fₛₒₗ portion nucleic |
| | acid sequence |
| <400>5 | Splice site and codon optimised F protein nucleic |
| | acid sequence |
| <400>6 | Splice site and codon optimised Fₛₒₗ portion nucleic |
| | acid sequence |
| <400>7 | F protein amino acid sequence |
| <400>8 | Fₛₒₗ portion amino acid sequence |
| <400>9-<400>553 | F protein amino acid decapeptides |
| <400>554 | P protein amino acid sequence |
| <400>555 | Natural P protein nucleic acid sequence |
| <400>556 | Optimised P protein nucleic acid sequence |
| <400>557 | N protein amino acid sequence |
| <400>558 | Natural N protein nucleic acid sequence |
| <400>559 | Optimised N protein nucleic acid sequence |
| <400>560 | SH protein amino acid sequence |
| <400>561 | Natural SH protein nucleic acid sequence |
| <400>562 | Optimised SH protein nucleic acid sequence |
| <400>563 | F protein cleavage site 1 aa sequence |
| <400>564 | F protein cleavage site 2 aa sequence |
| <400>565 | F protein variant |
| <400>566 | F protein variant nucleic acid sequence |
| <400>567 | F protein variant |
| <400>568 | F protein variant nucleic acid sequence |
| <400>569 | F protein intervening aa sequence |
| <400>570 | Poly (a) adenylation site |

### EXAMPLE 1

### Design of Synthetic Gene for RSV F Expression

Initial attempts to express the RSV F gene sequence in a soluble form (truncated at the transmembrane domain) proved unsuccessful in achieving high levels of expression. The sequence used in the expression vectors was called F.ₛₒₗ. (this differed from the viral sequence in 24/1575 nucleotides where restriction sites had been inserted to allow for easy mutagenesis—see FIG. 2b). The F viral sequence (F.ₛₒₗ.viral FIG. 2b) contained suboptimal codon usage for expression in mammalian cells. In addition, a possible eight 3′ splice sites were identified, including preceding lariat sequences at four positions. Poly (A) adenylation sites (AATAAA <400>570) were also identified at 4 positions. In addition, the F natural sequence like the viral sequence is approximately 65% AT rich. Most mammalian expressed genes are less than 50% AT rich. The DNA sequence encoding the transmembrane form of RSV F is also shown in FIG. 2a.

In an attempt to overcome poor expression levels in mammalian cells, a new F sequence was designed that:

(a) retained the same encoded amino acid sequence

(b) used whereever possible optimum codon usage

(c) removed all potential splice sites and poly A sites

(d) removed as many CG doublets as these may be methylation sites

(e) designed unique restriction sites to allow cassette mutagenesis

(f) sequence was checked by secondary structure and any large hairpin loops were destabilised by changing the sequence

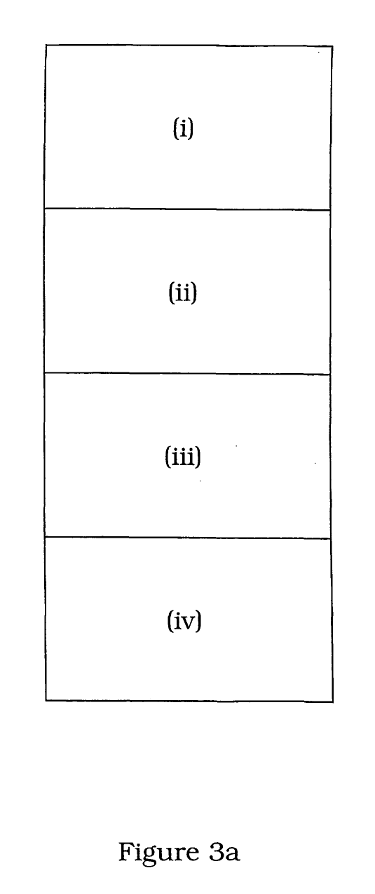

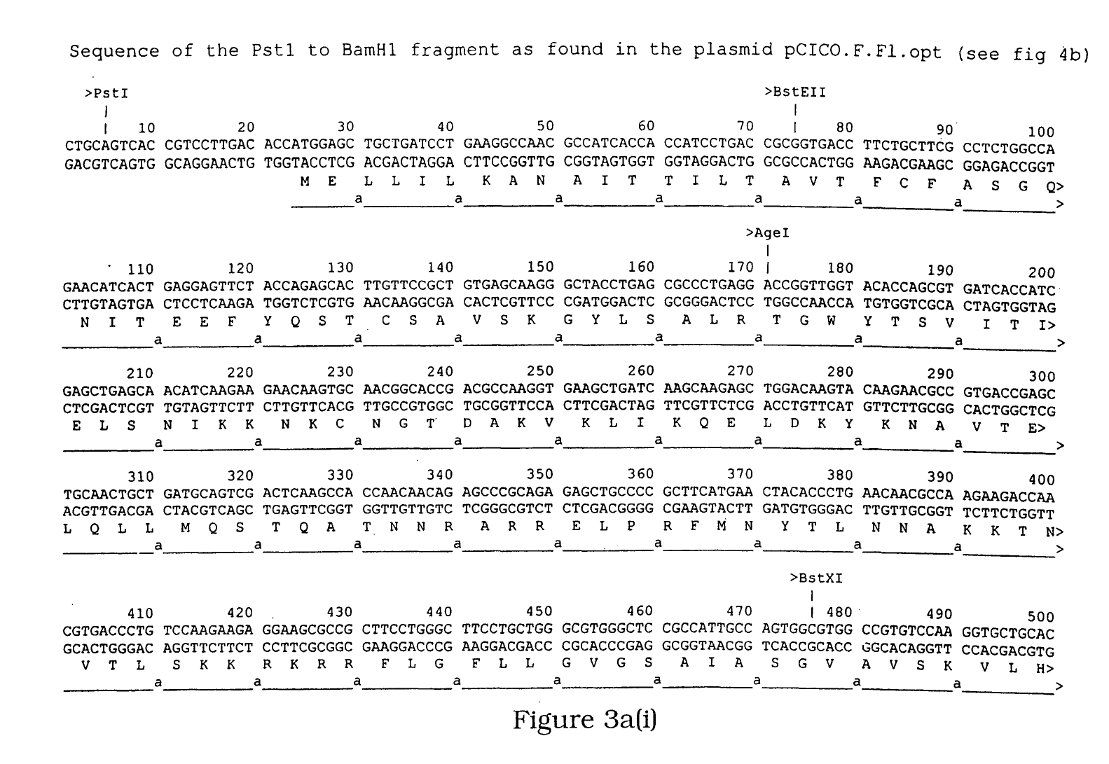

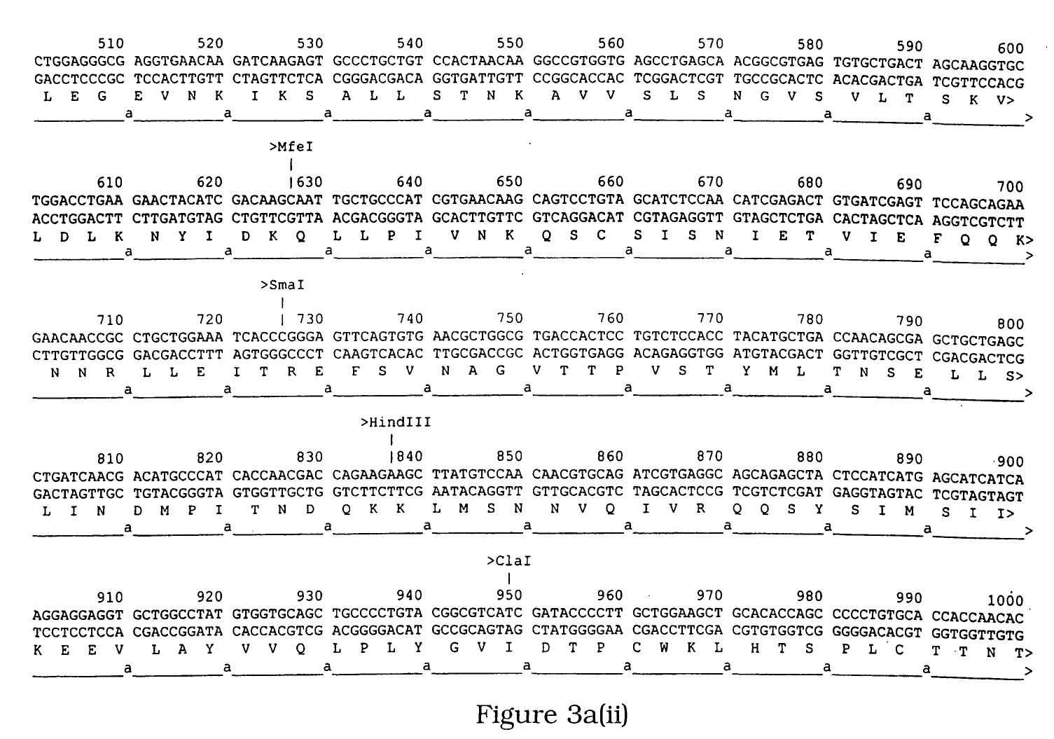

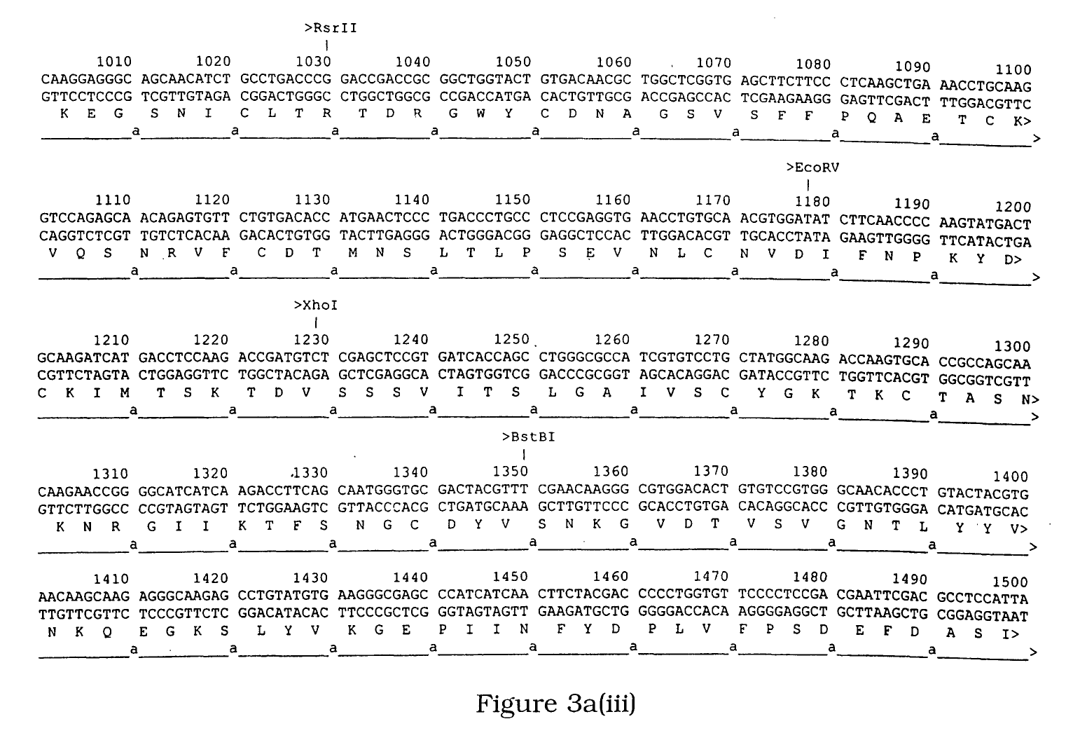

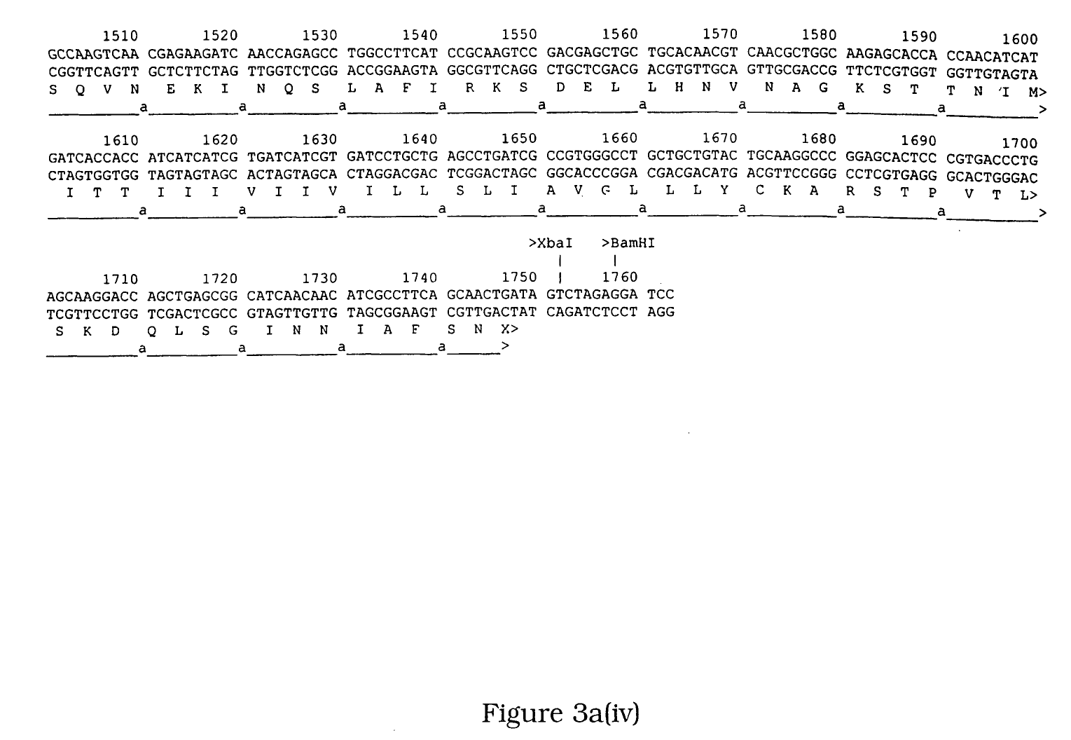

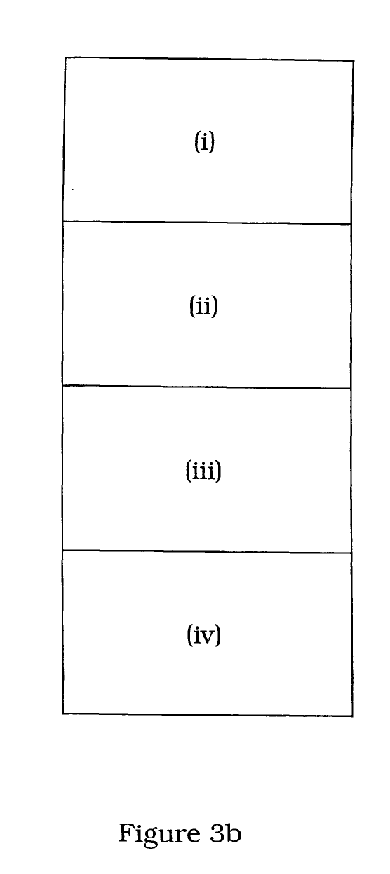

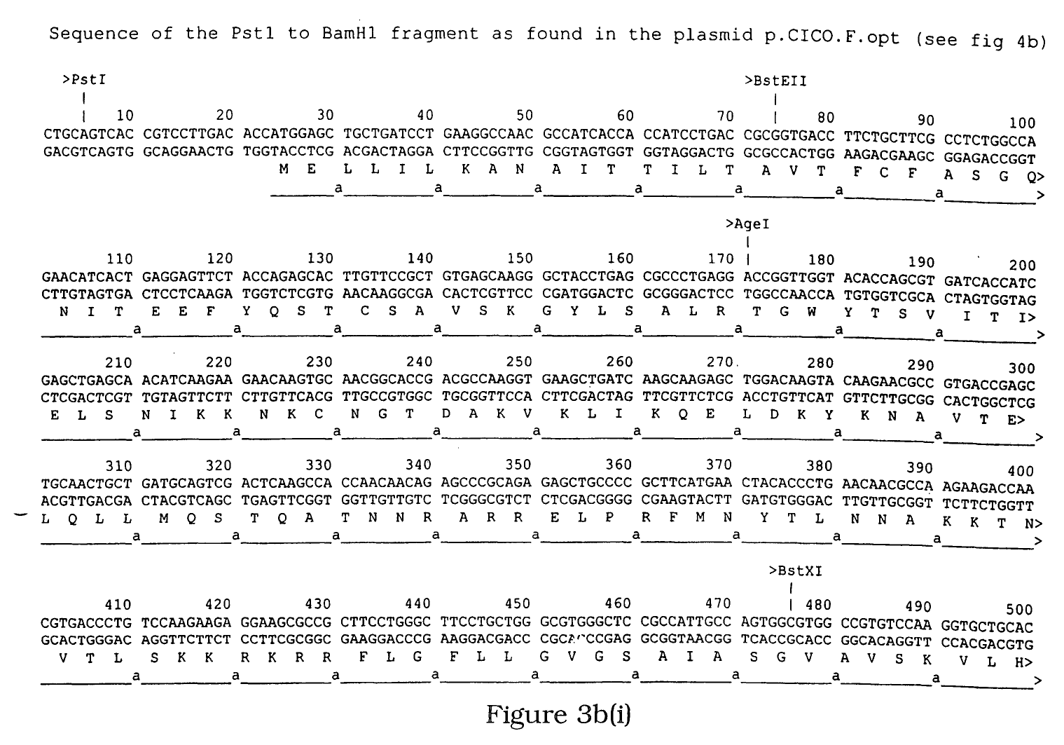

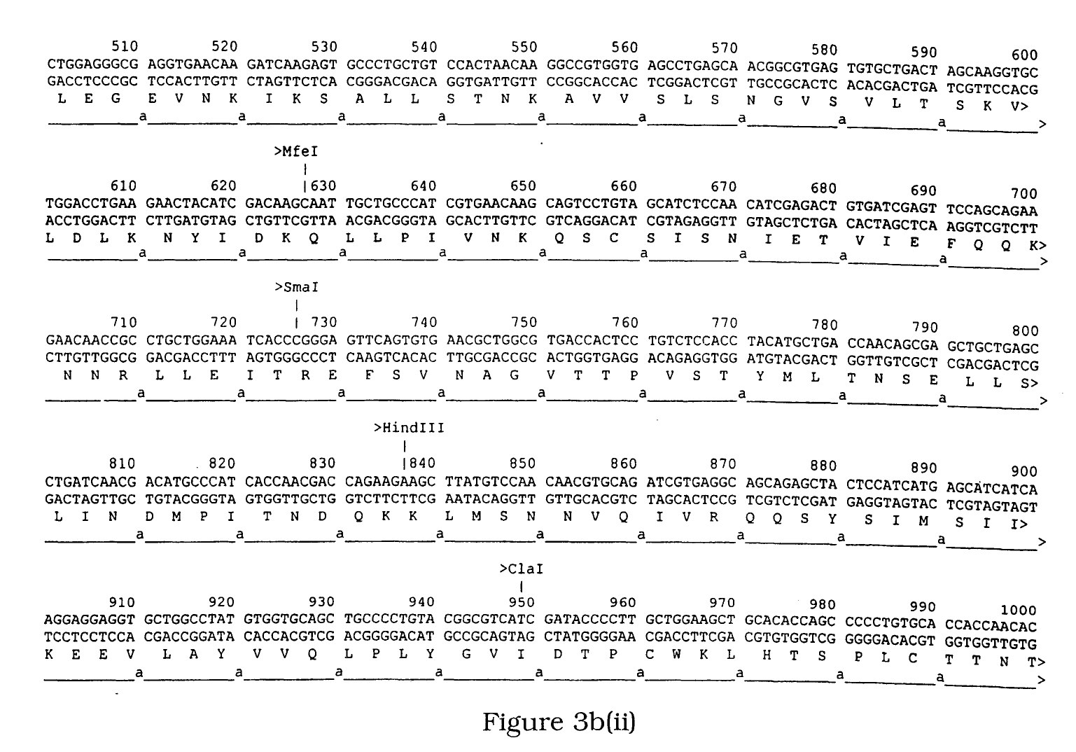

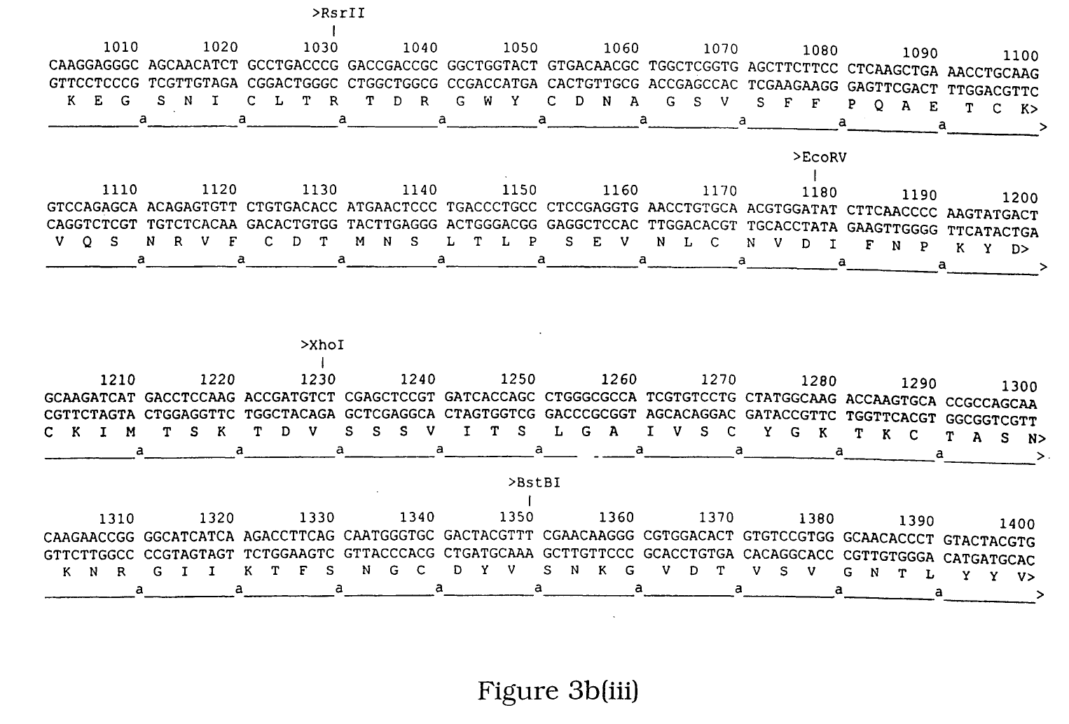

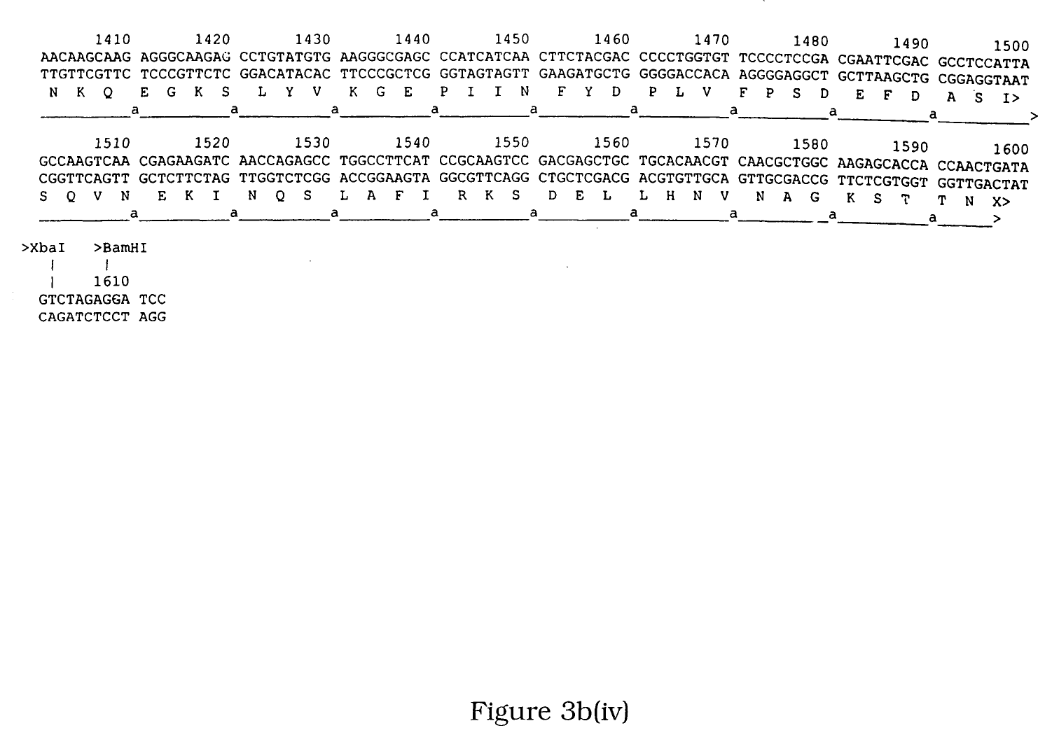

Sequences encoding a transmembrane version of F and the Fₛₒₗ protein are shown in FIGS. 3a and 3b respectively.

Both of these optimised sequences F.opt and F.ₛₒₗ.opt are compared to the viral sequence in FIGS. 2a and 2b.

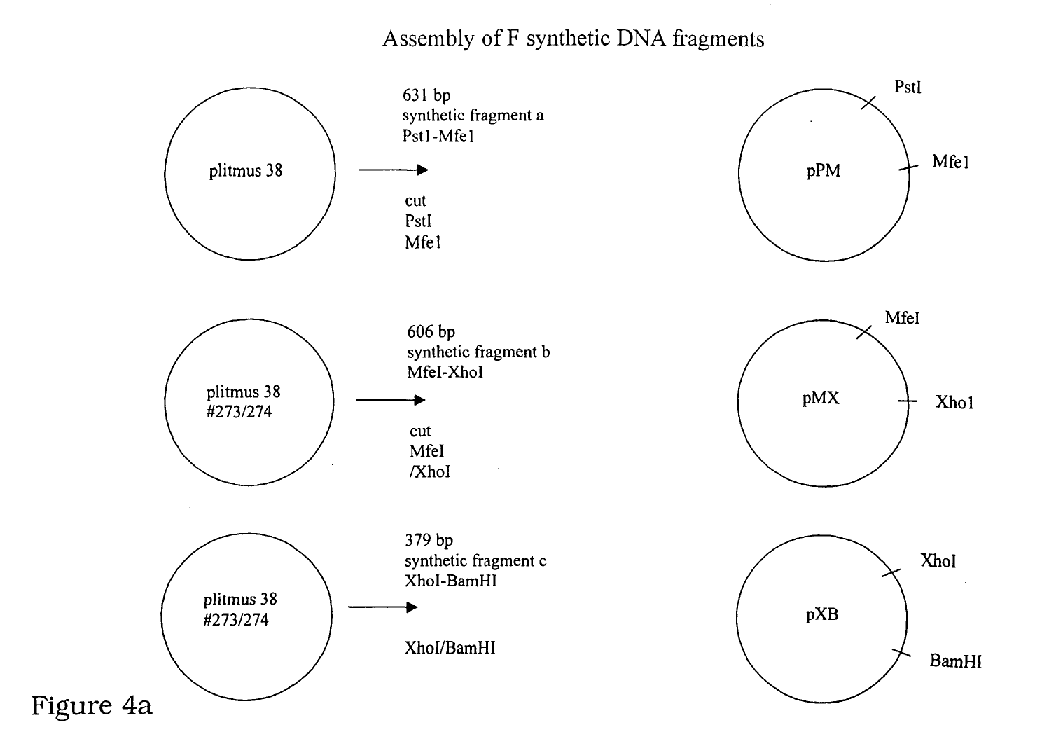

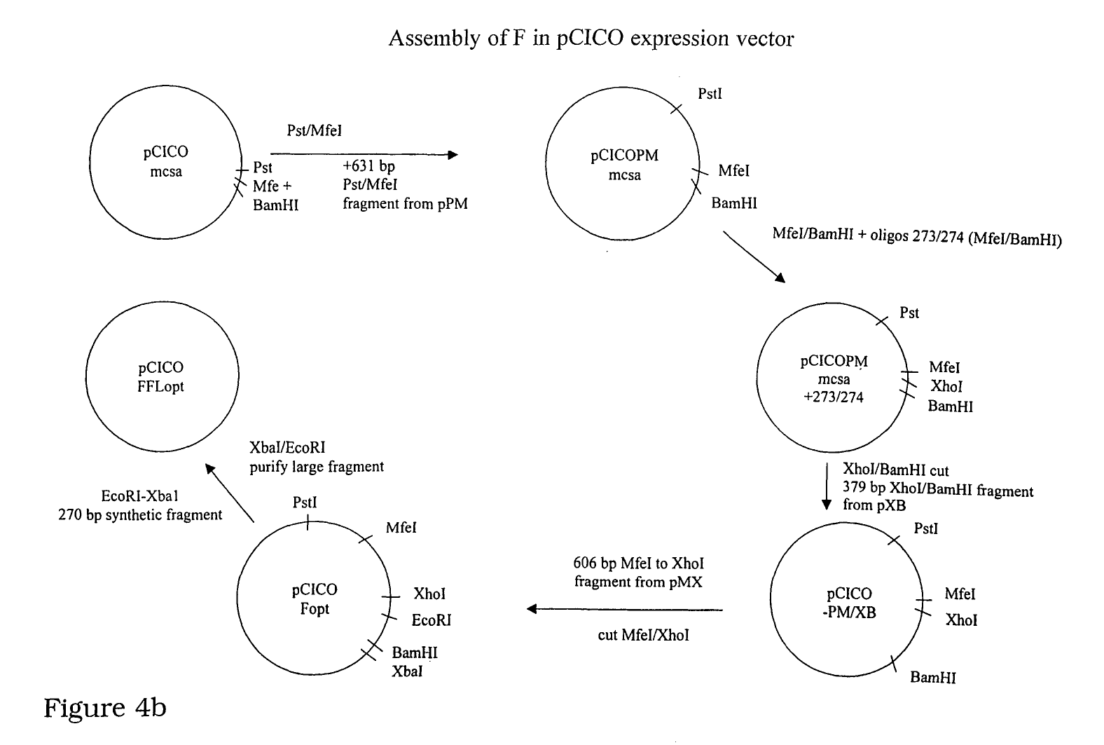

The synthetic DNA sequence Fopt (also referred to as F.ₛₒₗ.opt) was assembled and cloned as outlined in FIGS. 4a and 4b. In brief, single stranded synthetic DNA fragments of average length 60 bases were annealed and ligated together to produce three fragments

(1) a 6311 bp Pst 1-Mfe I fragment

(2) a 606 bp Mfe I-Xho I fragment

(3) a 379 bp Xho I-Bam HI fragment.

These gel purified fragments were cloned in pLitmus 38 or a derivative of pLITMUS (pLITMUS 273/279). Clones containing the correct sequence were used as a DNA source to assemble the full length gene as outlined in FIG. 4b. In brief the respective fragment Pst-Mfe I, Xho I-Bam HI and Mfe-Xho I were sequentially cloned into the CMV expression vector pCICO or its derivatives. [pCICO is a derivative of pJW4304 which contains a full length CMV promoter and the CMV authentic intron sequence preceding the Pst I site. The 3′ terminator used is derived from SV40 early region and this vector also contains the SV40 origin of replication. The plasmid is from the pUC series and contains an ampicillin resistance gene. (pJW4304 was obtained from J. Mullins Dept. of Microbiology, University of Washington, Chapman et al., NAR, 19:3979-3980, 1991)]. This produced the final clone pCICO.Fopt.

pCICO.Fopt was further modified by cloning in a 270 bp EcoRI-Xba I fragment (see FIG. 4b) which encodes the transmembrane and cytoplasmic domains of the RSV F protein. Again, the DNA sequence was optimised as for the soluble version See FIG. 2b for comparison of F.opt (Fopt FL sequence) and F (viral with a few additional restriction site changes) and F.viral (viral sequence). The resulting CMV expression plasmid is called pCICO.F.FL.opt. Note FL stands for the term full length and refers to a form of F that includes the transmembrane region and the cytoplasmic tail.

### EXAMPLE 2

### In Vitro Expression of RSV F Expression

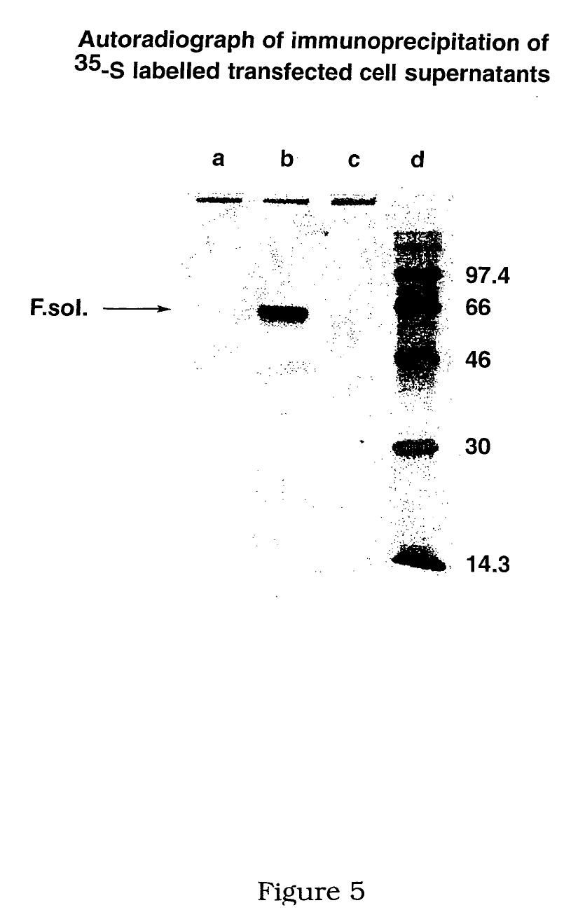

Vectors pCICO containing the F.ₛₒₗ.opt sequence (pCICO.Fopt) and the F.ₛₒₗ sequence (pCICO.FS3) were tested for expression by CaPO₄ precipitation in 293 cells. Cells in a 60 ml dish were transfected with 5 μg of plasmid and 0.5 μg of pVARNA. Cells were radioactively labelled with ³⁵S methionine and ³⁵S cystene 24 hours post transfection and the supernatants collected 5 hours after labelling. Supernatants were immunoprecipitated with a RSV F specific monoclonal antibody and the precipitates were analysed by polyacrylamide gel electrophoresis. Gels were subjected to fluorography, dried and exposed to X-ray film. FIG. 5 shows an autoradiograph comparing the amount of F in pCICO.FS3, pCICO.Fopt and control (mock-transfected) cells. Expression is much improved in the pCICO.Fopt transfected cells by at least 20 fold.

### EXAMPLE 3

### RSV Fusion Assay

293 cells were also transfected with the plasmid pCICO.F.FL.opt which contains the transmembrane spanning version of F. Cells transfected with this plasmid were observed 24-48 housrs post transfection to contain many large synetia and dying cells. Control cells were confluent. The F transfected cells look indistinguishable from RSV infected cells. Thus high level expression of F is all that is necessary for cell fusion to occur. This is markedly different to what is reported in the literature (Collins et al, Fields, and references within). This assay forms a useful screen for detecting F specific inhibitors of RSV fusion. Agents found by this assay are also useful for inhibiting RSV replication.

### EXAMPLE 4

### RSV Second Cleavage Site Mutants

The RSV F protein sequence at amino acid singular numbers 106-109, contains the sequence RARR. As shown in FIG. 1c, this potential cleavage site is contained within the F2 sub-unit of the F protein. When the F protein is expressed in mammalian cells, proteolytic cleavage occurs at two sites being site 1 (KKRKRR amino acids 131-136) which was previously identified and the previously unknown site 2 (RARR amino acids 106-109).

The site RARR was mutated to GQGR in the expression plasmid pCICO.FL.Fopt to give rise to the plasmid pCICO.F.FL.S2-2. Transfection of this plasmid into 293 cells revealed cleavage at site 1 but not at site 2 as expected. This was detected by a larger size F2 sub-unit (˜30K versus 1 SK) in the S2-2 mutant than in the wild type. The size of the protein between site 2 and site 1 would be expected to be 10-12K (25 amino acids plus two NH₂-linked glycosylation sites). It was surprisingly noted that no evidence of fusion was seen in the 293 cells transfected with the S2-2 mutant plasmid of wild type. This evidence would suggest that cleavage at both site 1 and site 2 is necessary for cleavage. Note that in additional experiments, mutation of site 1 (KKRKRR) to GGKQGR, produced a mutant showing no fusion activity.

In the next experiments the issue of whether the sequence between sites 1 and 2 were necessary for fusion was addressed. A mutant was constructed by standard techniques (cassette mutagenesis) in which amino acids 106-130 were deleted. This mutant is designated delta 106-130. Transfection of 293 cells with an expression plasmid containing this mutant (pCICO.FLFΔ106-130) showed that fusion did occur. This fusion was phenotypically different from wild type in that only small syncytia were visible, suggesting that the ability of the RSV F protein to initiate or perform fusion had been attenuated.

### EXAMPLE 5

### Expression of Natural F-V-F Optimised Sequence

Cloning of RSVA2 F cDNA

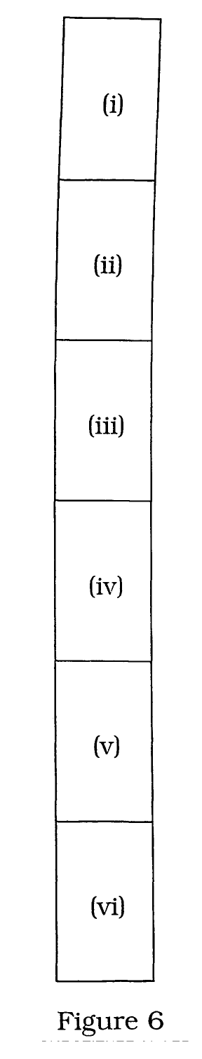

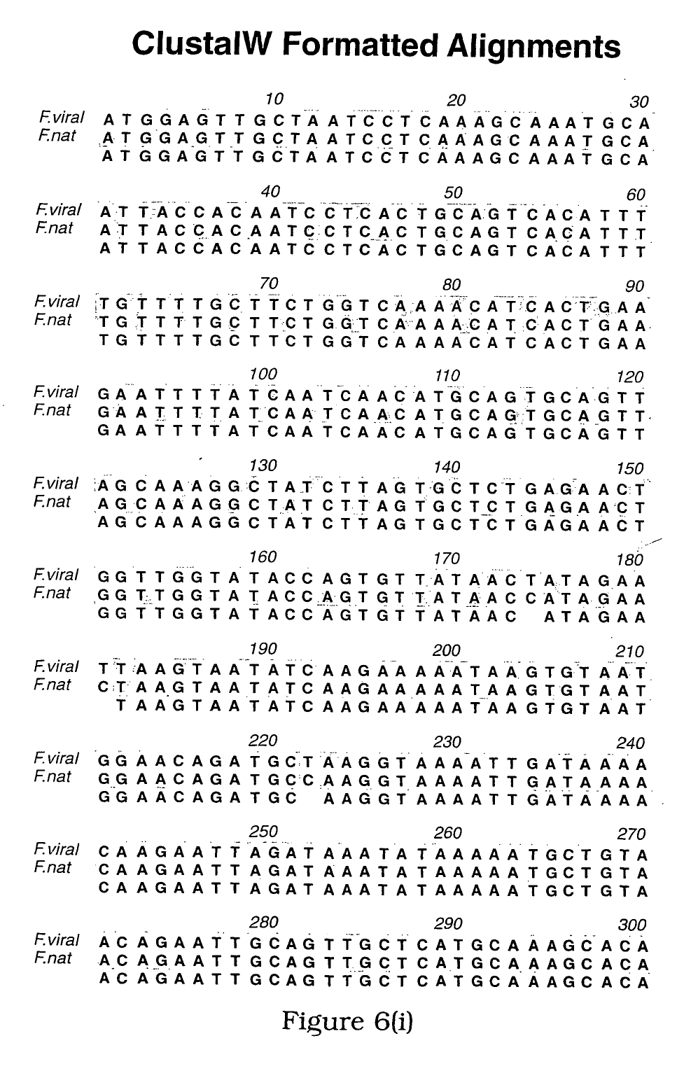

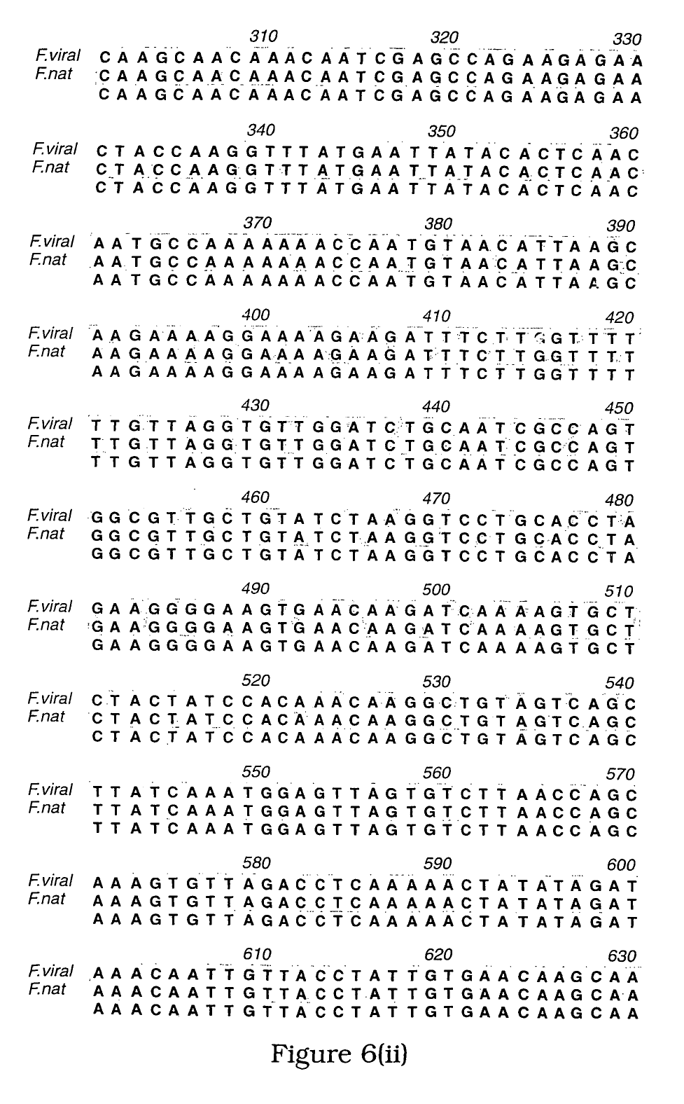

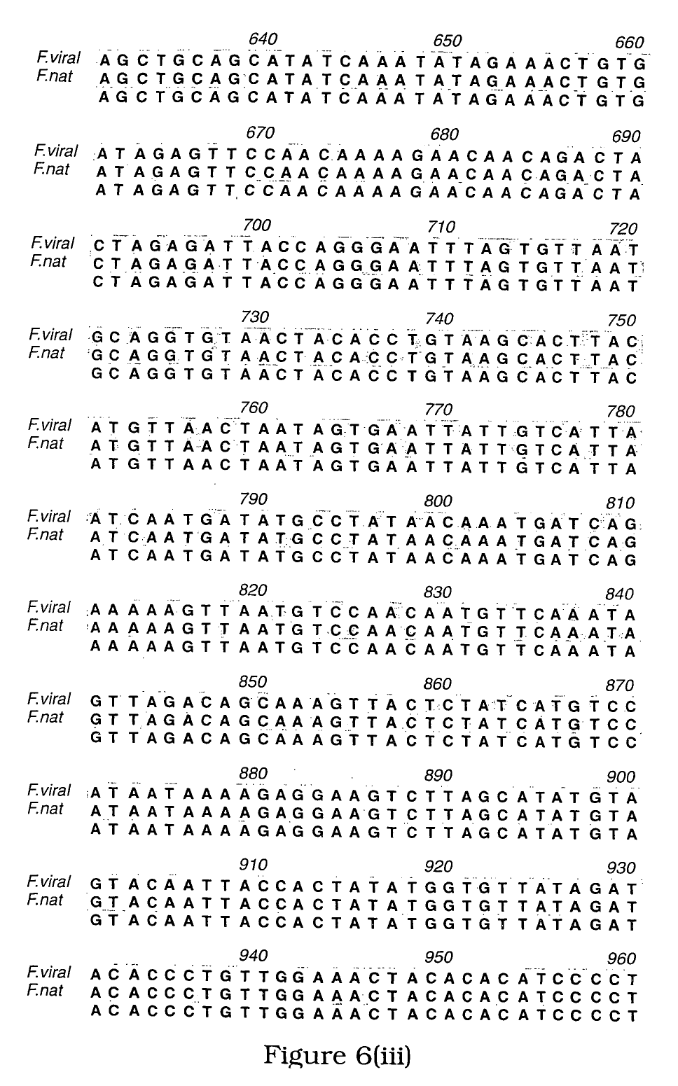

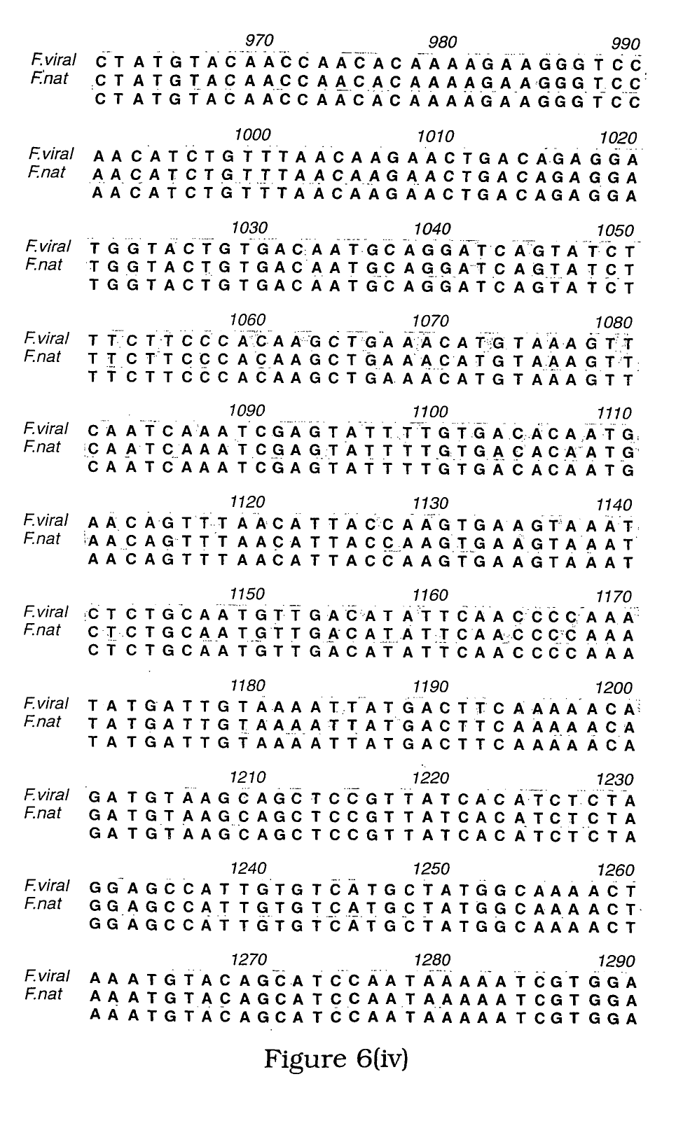

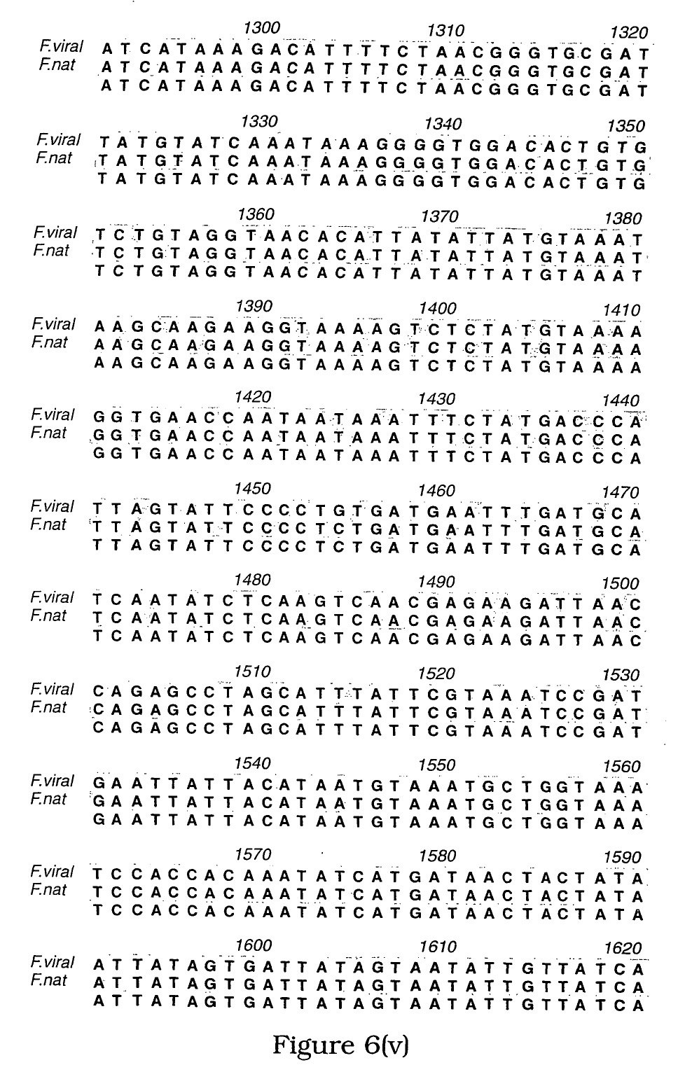

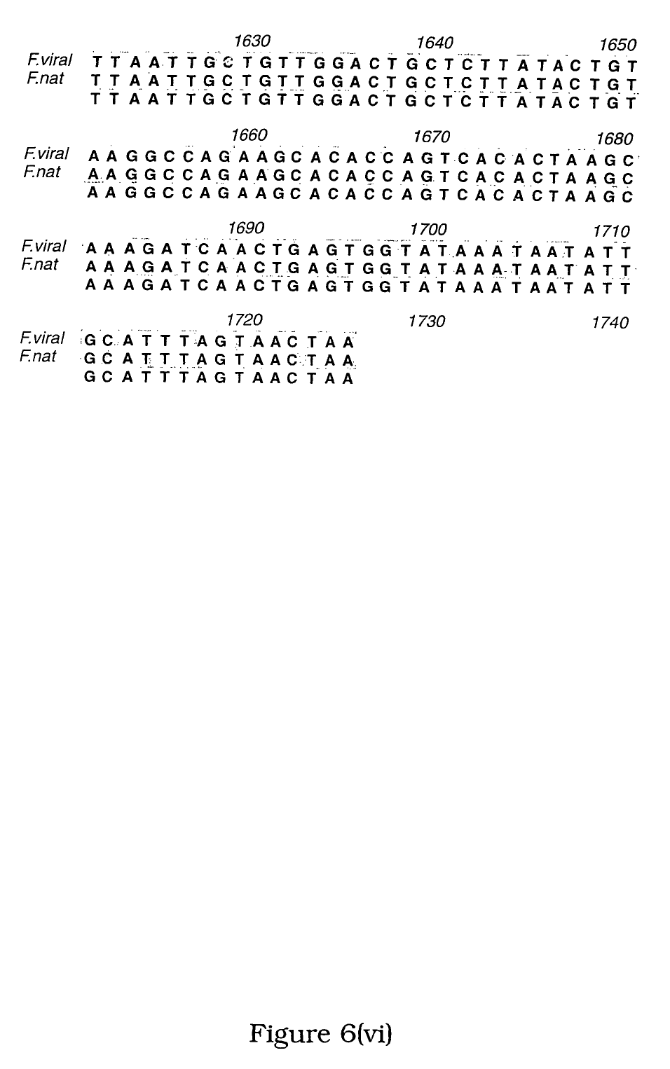

RNA prepared from RSVA2 infected Hep-2 cells was used as a source of rSV A2 F mRNA. RT-PCR (reverse transcriptase PCR) using 5′- and 3′-end primers was used to prepare cDNA encoding RSV A2 F according to standard methods. PCR products were subcloned into standard vectors. Sequencing of many clones revealed a consensus sequence for the F gene of RSV A2. This sequence is shown in FIG. 6 as F.nat and compared to F.viral. The F.nat sequence differs at nt 174 and 222. Both of these T to C changes do not result in amino acid changes. A pCICO vector containing the F.nat sequence (called pCICO.F.nat) was assembled from a synthetic Pst1 to Acc1 157 bp fragment ligated to a 445 bp Acc1 to Mfe I fragment and a 1125 bp Mfe 1 to Xba 1 fragment derived from independent RT-PCR RSVA2 F cDNA clones. The synthetic fragment was used to make the addition of extra 5′-untranslated sequences not present in the PCR products. The 5′-untranslated sequence is 5′-CTGCAGTCACCGTCCTTGA-CACC-3′ (<400>571) and includes a Pst 1 site. This sequence is added just 5′ to the initiator ATG in the following constructions pCICO.F.nat and the previously described pCICO.F.FL.opt. The Acc1 to Mfe 1 and Mfe 1 to Xba1 fragments were derived from independent RT-PCR RSVA2 F cDNA clones. The sequnce F.nat encodes the same 574 amino acid sequence as shown in FIG. 1.

Expression of pCICO.F.FL.opt Versus pCICO.F.nat

293 cells were transfected with plasmids pCICO.F.FL.opt, pCICO.F.nat and a control as described in example 2. Cells were harvested at 24, 48 and 72 hours post transfection in cell lysis buffer. The amount of F protein in these samples was measured by Western blot analysis using standard techniques. The primary antibody called 1 SB2, is a mouse monoclonal antibody that recognizes the F1 protein. A proteolytic breakdown product of

F1 called F1′ is also recognized by this antibody. The western blots were developed using a secondary anti-mouse horseradish peroxidase antibody and a light emitting substrate according to standard procedures.

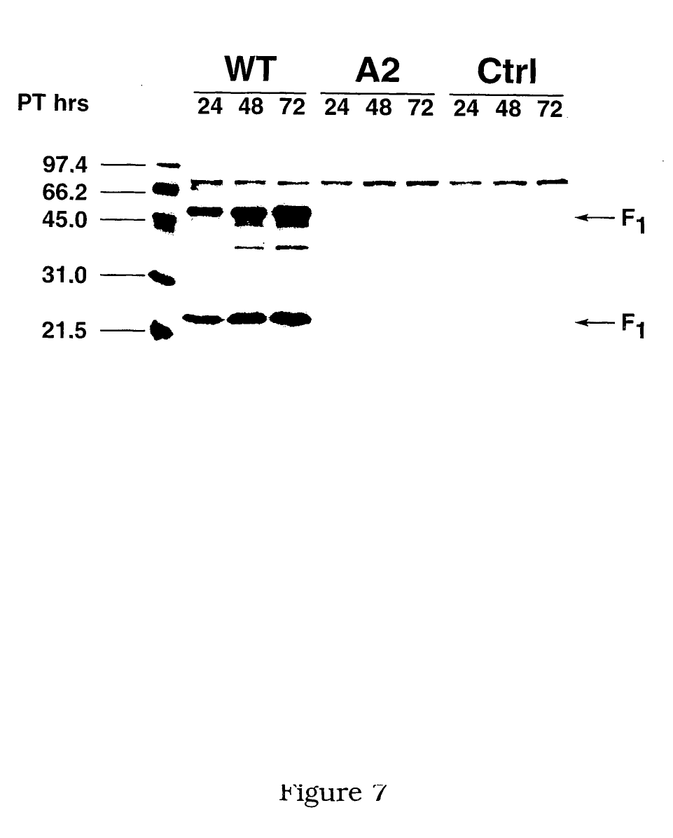

The results of these experiments are shown in FIG. 7. Lanes labelled WT refer to samples from cells transfected with pCICO.F.FL.opt: A2 lanes refer to samples from cells transfected with pCICO.F.nat and Ctrl lanes are from cells transfected with control plasmids lacking either F sequence. F protein (F1 and F1′) is only observed in WT lanes indicating that the F expression level in cells transfected with pCICO.F.F1.opt is far superior to those transfected with pCICO.F.nat.

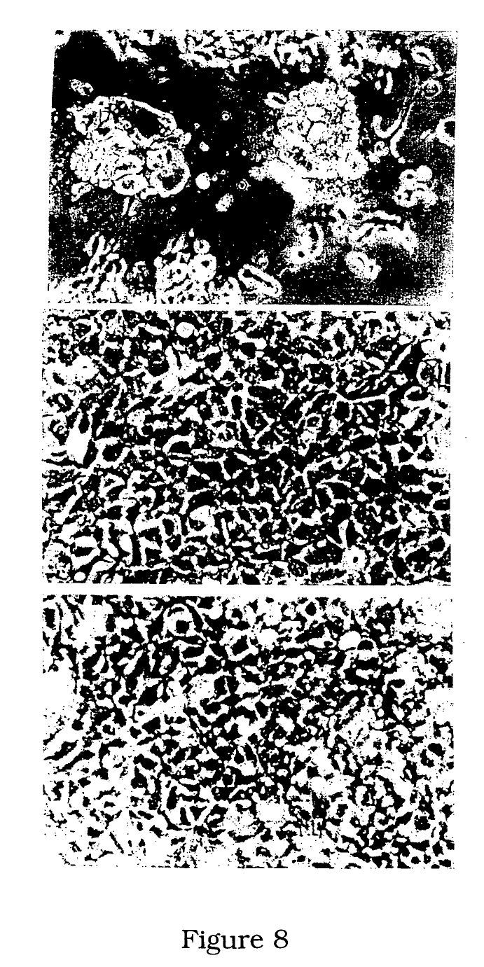

In parallel to the above experiments 293 cells were transfected with the same three plasmids and observed microscopically for signs of cell to cell fusion (syncytia formation). In three parallel experiments only cells tranfected with pCICO.F.FL.opt show any cell to cell fusion. At 72 hours post transfection between 75 to 100% of cells were involved in syncytia in pCICO.FL.opt transfected cells. No fusion is observed in either the pCICO.F.nat or Ctrl transfected cells (see FIG. 8).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

BIBLIOGRAPHY

Chapman, B. S., Thayer, R. M., Vincent, K. A. and Haigwood, N. L., (1991), Nucleic Acids Res., 19(14):3979-3986

Collins et al., In: Fields Virology Third Edition (Eds), Fields B., Knipe D., and Howley P., Lippincott Raven, Philadelphia, USA, (1996) pp 1313-1352

Gardsvoll, H., Dano, K., and Ploug, M., (1999), J Biol Chem, 274(53):37995-38003

Goodson, R. J., Doyle, M. V., Kaufman, S. E., Rosenberg, S., (1994), Proc. Natl. Acad. Sci. USA, 91(15):7129-7133

Haas, J., Parke E. C. and Seed B., (1996), Current Biology, 6:315-324

Hoyer-Hansen, G., Behrendt, N., Ploug, M., Dano, K., and Preissner, K. T., (1997), FEBS Lett, 420(1):79-85

Kuhnle et al., (1998), Journal of Virology, 72(5):3804-3811

Lathe, R., (1985), J. Mol. Biol., 183:1-12

Lopez et al., (1998), J. Virol., 72:6922-6928

Navaza, J., (1994), Acta Cryst, A50:157-163

Ploug, M., (1998). Biochemistry, 37(47):16494-16505

Ploug, M., Ellis, V., and Dano, K., (1994), Biochemistry, 33(30):8991-8997

Ploug, M., Ostergaard, S., Hansen, L. B., Holm, A., and Dano, K., (1998), Biochemistry, 37(11):3612-3622

Ploug, M., Rahbek-Nielsen, H., Ellis, V., Roepstorff, P., and Dano, K., (1995), Biochemistry, 34(39): 12524-12534

Steiner, D., (1998), Current Opinion in Chemical Biology, 2:31-39

Stephens, R. W., Bokman, A. M., Myohanen, H. T., Reisberg, T., Tapiovaara, H., Pedersen, N., Grondahl-Hansen, J., Llinas, M., and Vaheri, A., (1992), Biochemistry, 31:7572-7579

Terrett, N., (2000), Drug Discov Today, 5(5):211-212

1. A method of facilitating production of a protein or derivative thereof from a negative sense single stranded RNA virus, said method comprising expressing in a host cell a nucleic acid molecule encoding said protein or derivative thereof, the nucleotide sequence of which nucleic acid molecule is optimised for expression by a eukaryotic cell.

2. The method according to claim 1 wherein said virus is a virus from the family Paramyxoviridae.

3. The method according to claim 2 wherein said virus is of the sub-family Pneumovirinae.

4. The method according to claim 3 wherein said virus is respiratory syncytial virus.

5. The method according to any one of claims 1-4 wherein said protein directly or indirectly facilitates fusion of any one or more viral components with any one or more host cells components.

6. The method according to claim 5 wherein said protein is a F protein or derivative thereof.

7. The method according to claim 6 wherein said derivative is the Fₛₒₗ fragment.

8. The method according to claim 5 wherein said protein is an N protein or derivative thereof.

9. The method according to claim 5 wherein said protein is a P protein or derivative thereof.

10. The method according to claim 5 wherein said protein is a SH protein or derivative thereof.

11. The method according to any one of claims 1-10 wherein said eukaryotic host cell is a mammalian cell.

12. The method according to claim 11 wherein said mammalian cell is a 293 cell.

13. The method according to claim 11 wherein said mammalian cell is a Chinese Hamster Ovary Cell.

14. The method according to any one of claims 11-113 wherein said optimisation is codon optimisation and/or nucleotide splice site deletion.

15. The method according to claim 14, wherein said codon optimisation comprises modification of at least one A and/or T comprising codon to express G and C, respectively and said splice site deletion comprises deletion of at least one RNA splice site.

16. The method according to claim 14 or 15 wherein said optimised protein encoding nucleic acid molecule further comprises one or more endonuclease restriction sites.

17. The method according to any one of claims 14-16 wherein said optimised F protein encoding nucleic acid sequence corresponds to the sequence defined by <400>3 or derivative thereof.

18. The method according to any one of claims 14-16 wherein said optimised F protein encoding nucleic acid sequence corresponds to the sequence defined by <400>5 or derivative thereof.

19. The method according to any one of claims 14-16 wherein said optimised FSOL protein encoding nucleic acid sequence corresponds to the sequence defined by <400>4 or derivative thereof.

20. The method according to any one of claims 14-16 wherein said optimised FSOL protein encoding nucleic acid sequence corresponds to the sequence defined by <400>6 or derivative thereof.

21. The method according to any one of claims 14-16 wherein said optimised P protein encoding nucleic acid sequence corresponds to the sequence defined by <400>556 or derivative thereof.

22. The method according to any one of claims 14-16 wherein said optimised N protein encoding nucleic acid sequence correspond to the sequence defined by <400>559 or derivative thereof.

23. The method according to any one of claims 14-16 wherein said SH protein encoding nucleic acid sequence corresponds to the sequence defined by <400>562 or derivative thereof.

24. An optimised nucleic acid molecule or derivative thereof as described in any one of claims 1-23.

25. A protein molecule encoded by the optimised nucleic acid molecule of claim 24 or derivative, equivalent, analogue or mimetic thereof.

26. A method of regulating the functional activity of a viral F protein, which protein in its non-fully functional form comprises an F2 portion linked, bound or otherwise associated with an F1 portion, which F2 portion comprises an intervening peptide sequence, said method comprising modulating cleavage of said intervening peptide sequence wherein excision of at least part of said intervening sequence from said non-fully functional form of said protein up-regulates F protein functional activity.

27. The method according to claim 26 wherein said method comprises expressing in a host cell a nucleic acid molecule encoding said protein or derivative thereof, the nucleotide sequence of which nucleic acid molecule is optimised for expression by a eukaryotic cell.

28. The method according to claim 27 wherein said virus is a virus from the family Paramyxoviridae.

29. The method according to claim 28 wherein said virus is of the sub-family Pneumovirinae.

30. The method according to any one of claims 26-29 wherein said cleavage events occur at the cleavage sites defined by the peptide sequence RARR (<400>564) and KKRKRR (<400>563).

31. The method according to any one of claims 26-29 wherein said F protein, in its non-fully functional form, comprises the structure:
X₁X₂ X₃
wherein:
X₁ comprises the non-intervening peptide sequence region of the F2 portion;
X₂ comprises the intervening peptide sequence region of the F₂ portion; and
X₃ comprises the F1 portion

32. The method according to claim 31 wherein said cleavage events occur at the cleavage sites defined by the peptide sequence RARR (<400>564) and KKRKRR (<400>563).

33. The method according to any one of claims 26-32 wherein said regulation is down-regulation.

34. A method for detecting an agent capable of regulating the functional activity of a viral F protein or derivative thereof said method comprising contacting a eukaryotic cell expressing an optimised nucleic acid molecule in accordance with the method of any one of claims 1-23 with a putative modulatory agent and detecting an altered expression phenotype and/or functional activity.

35. The method of claim 34 wherein said viral F protein is a non-fully functional form of said protein and wherein said agent modulates cleavage of the intervening peptide sequence.

36. A method for detecting an agent capable of regulating the functional activity of a viral F protein or derivative thereof said method comprising contacting a host cell, which host cell expresses a nucleic acid molecule encoding the non-fully functional form of said viral F protein derivative thereof, with a putative modulatory agent and detecting an altered expression phenotype and/or altered functional activity wherein said agent modulates the site 2 cleavage event.

37. A method for analysing, designing and/or modifying an agent capable of interacting with a viral F protein or derivative thereof and modulating at least one functional activity associated with said protein, which protein is produced in accordance with the method of any one of claims 1-23, said method comprising contacting said F protein or derivative thereof with a putative agent and assessing the degree of interactive complementarity of said agent with said protein.

38. The method of claim 37 wherein said virus is a virus from the family Paramyxoviridae.

39. The method according to claim 38 wherein said virus is of the sub-family Pneumovirinae.

40. The method according to claim 39 wherein said virus is respiratory syncytial virus.

41. An agent capable of interacting with a viral F protein and modulating at least one functional activity associated with said viral protein.

42. The agent according to claim 41 wherein said agent is an antagonist which interacts with a sequence selected from:

| | | | |
|---|---|---|---|
| CFASGQNITE | <400>9 | PASGQNITEE | <400>10 |
| ASGQNITEEF | <400>11 | SGQNITEEFY | <400>12 |
| GQNITEEFYQ | <400>13 | QNITEEFYQS | <400>14 |
| NITEEFYQST | <400>15 | ITEEFYQSTC | <400>16 |
| TEEFYQSTCS | <400>17 | EEFYQSTCSA | <400>18 |
| EFYQSTCSAV | <400>19 | FYQSTCSAVS | <400>20 |
| YQSTCSAVSK | <400>21 | QSTCSAVSKG | <400>22 |
| STCSAVSKGY | <400>23 | TCSAVSKGYL | <400>24 |
| CSAVSKGYLS | <400>25 | SAVSKGYLSA | <400>26 |
| AVSKGYLSAL | <400>27 | VSKGYLSALR | <400>28 |
| SKGYLSALRT | <400>29 | KGYLSALRTG | <400>30 |
| GYLSALRTGW | <400>31 | YLSALRTGWY | <400>32 |
| LSALRTGWYT | <400>33 | SALRTGWYTS | <400>34 |
| ALRTGWYTSV | <400>35 | LRTGWYTSVI | <400>36 |
| RTGWYTSVIT | <400>37 | TGWYTSVITI | <400>38 |
| GWYTSVITIE | <400>39 | WYTSVITIEL | <400>40 |
| YTSVITIELS | <400>41 | TSVITIELSN | <400>42 |
| SVITIELSNI | <400>43 | VITIELSNIK | <400>44 |
| ITIELSNIKK | <400>45 | TIELSNIKKN | <400>46 |
| IELSNIKKNK | <400>47 | ELSNIKKNKC | <400>48 |
| LSNIKKNKCN | <400>49 | SNIKKNKCNG | <400>50 |
| NIKKNKCNGT | <400>51 | IKKNKCNGTD | <400>52 |
| KKNKCNGTDA | <400>53 | KNKCNGTDAK | <400>54 |
| NKCNGTDAKV | <400>55 | KCNGTDAKVK | <400>56 |
| CNGTDAKVKL | <400>57 | NGTDAKVKLI | <400>58 |
| GTDAKVKLIK | <400>59 | TDAKVKLIKQ | <400>60 |
| DAKVKLIKQE | <400>61 | AKVKLIKQEL | <400>62 |
| KVKLIKQELD | <400>63 | VKLIKQELDK | <400>64 |
| KLIKQELDKY | <400>65 | LIKQELDKYK | <400>66 |
| IKQELDKYKN | <400>67 | KQELDKYKNA | <400>68 |
| QELDKYKNAV | <400>69 | ELDKYKNAVT | <400>70 |
| LDKYKNAVTE | <400>71 | DKYKNAVTEL | <400>72 |
| KYKNAVTELQ | <400>73 | YKNAVTELQL | <400>74 |
| KNAVTELQLL | <400>75 | NAVTELQLLM | <400>76 |
| AVTELQLLMQ | <400>77 | VTELQLLMQS | <400>78 |
| TELQLLMQST | <400>79 | ELQLLMQSTQ | <400>80 |
| LQLLMQSTQA | <400>81 | QLLMQSTQAT | <400>82 |
| LLMQSTQATN | <400>83 | LMQSTQATNN | <400>84 |
| MQSTQATNNR | <400>85 | QSTQATNNRA | <400>86 |
| STQATNNRAR | <400>87 | TQATNNRARR | <400>88 |
| QATNNRARRE | <400>89 | ATNNRARREL | <400>90 |
| TNNRARRELP | <400>91 | NNRARRELPR | <400>92 |
| NRARRELPRF | <400>93 | RARRELPRFM | <400>94 |
| ARRELPRFMN | <400>95 | RRELPRFMNY | <400>96 |
| RELPRFMNYT | <400>97 | ELPRFMNYTL | <400>98 |
| LPRFMNYTLN | <400>99 | PRFMNYTLNN | <400>100 |
| RFMNYTLNNA | <400>101 | FMNYTLNNAK | <400>102 |
| MNYTLNNAKK | <400>103 | NYTLNNAKKT | <400>104 |
| YTLNNAKKTN | <400>105 | TLNNAKKTNV | <400>106 |
| LNNAKKTNVT | <400>107 | NNAKKTNVTL | <400>108 |
| NAKKTNVTLS | <400>109 | AKKTNVTLSK | <400>110 |
| KKTNVTLSKK | <400>111 | KTNVTLSKKR | <400>112 |
| TNVTLSKKRK | <400>113 | NVTLSKKRKR | <400>114 |
| VTLSKKRKRR | <400>115 | TLSKKRKRRF | <400>116 |
| LSKKRKRRFL | <400>117 | SKKRKRRFLG | <400>118 |
| KKRKRRFLGF | <400>119 | KRKRRFLGFL | <400>120 |
| RKRRFLGFLL | <400>121 | KRRFLGFLLG | <400>122 |
| RRFLGFLLGV | <400>123 | RFLGFLLGVG | <400>124 |
| FLGFLLGVGS | <400>125 | LGFLLGVGSA | <400>126 |
| GFLLGVGSAI | <400>127 | FLLGVGSAIA | <400>128 |
| LLGVGSAIAS | <400>129 | LGVGSAIASG | <400>130 |
| GVGSAIASGV | <400>131 | VGSAIASGVA | <400>132 |
| GSAIASGVAV | <400>133 | SAIASGVAVS | <400>134 |
| AIASGVAVSK | <400>135 | IASGVAVSKV | <400>136 |
| ASGVAVSKVL | <400>137 | SGVAVSKVLH | <400>138 |
| GVAVSKVLHL | <400>139 | VAVSKVLHLE | <400>140 |
| AVSKVLHLEG | <400>141 | VSKVLHLEGE | <400>142 |
| SKVLHLEGEV | <400>143 | KVLHLEGEVN | <400>144 |
| VLHLEGEVNK | <400>145 | LHLEGEVNKI | <400>146 |
| HLEGEVNKIK | <400>147 | LEGEVNKIKS | <400>148 |
| EGEVNKIKSA | <400>149 | GEVNKIKSAL | <400>150 |
| EVNKIKSALL | <400>151 | VNKIKSALLS | <400>152 |
| NKIKSALLST | <400>153 | KIKSALLSTN | <400>154 |
| IKSALLSTNK | <400>155 | KSALLSTNKA | <400>156 |
| SALLSTNKAV | <400>157 | ALLSTNKAVV | <400>158 |
| LLSTNKAVVS | <400>159 | LSTNKAVVSL | <400>160 |
| STNKAVVSLS | <400>161 | TNKAVVSLSN | <400>162 |
| NKAVVSLSNG | <400>163 | KAVVSLSNGV | <400>164 |
| AVVSLSNGVS | <400>165 | VVSLSNGVSV | <400>166 |
| VSLSNGVSVL | <400>167 | SLSNGVSVLT | <400>168 |
| LSNGVSVLTS | <400>169 | SNGVSVLTSK | <400>170 |
| NGVSVLTSKV | <400>171 | GVSVLTSKVL | <400>172 |
| VSVLTSKVLD | <400>173 | SVLTSKVLDL | <400>174 |
| VLTSKVLDLK | <400>175 | LTSKVLDLKN | <400>176 |
| TSKVLDLKNY | <400>177 | SKVLDLKNYI | <400>178 |
| KVLDLKNYID | <400>179 | VLDLKNYIDK | <400>180 |
| LDLKNYIDKQ | <400>181 | DLKNYIDKQL | <400>182 |
| LKNYIDKQLL | <400>183 | KNYIDKQLLP | <400>184 |
| NYIDKQLLPI | <400>185 | YIDKQLLPIV | <400>186 |
| IDKQLLPIVN | <400>187 | DKQLLPIVNK | <400>188 |
| KQLLPIVNKQ | <400>189 | QLLPIVNKQS | <400>190 |
| LLPIVNKQSC | <400>191 | LPIVNKQSCS | <400>192 |
| PIVNKQSCSI | <400>193 | IVNKQSCSIS | <400>194 |
| VNKQSCSISN | <400>195 | NKQSCSISNT | <400>196 |
| KQSCSISNIE | <400>197 | QSCSISNIET | <400>198 |
| SCSISNIETV | <400>199 | CSISNIETVI | <400>200 |
| SISNIETVIE | <400>201 | ISNIETVIEF | <400>202 |
| SNIETVIEFQ | <400>203 | NIETVIEFQQ | <400>204 |
| IETVIEFQQK | <400>205 | ETVIEFQQKN | <400>206 |
| TVIEFQQKNN | <400>207 | VIEFQQKNNR | <400>208 |
| IEFQQKNNRL | <400>209 | EFQQKNNRLL | <400>210 |
| FQQKNNRLLE | <400>211 | QQKNNRLLEI | <400>212 |
| QKNNRLLEIT | <400>213 | KNNRLLEITR | <400>214 |
| NNRLLEITRE | <400>215 | NRLLEITREF | <400>216 |
| RLLEITREFS | <400>217 | LLEITREFSV | <400>218 |
| LEITREFSVN | <400>219 | EITREFSVNA | <400>220 |
| TTREFSVNAG | <400>221 | TREFSVNAGV | <400>222 |
| REFSVNAGVT | <400>223 | EFSVNAGVTT | <400>224 |
| FSVNAGVTTP | <400>225 | SVNAGVTTPV | <400>226 |
| VNAGVTTPVS | <400>227 | NAGVTTPVST | <400>228 |
| AGVTTPVSTY | <400>229 | GVTTPVSTYM | <400>230 |
| VTTPVSTYML | <400>231 | TTPVSTYMLT | <400>232 |
| TPVSTYMLTN | <400>233 | PVSTYMLTNS | <400>234 |
| VSTYMLTNSE | <400>235 | STYMLTNSEL | <400>236 |
| TYMLTNSELL | <400>237 | YMLTNSELLS | <400>238 |
| MLTNSELLSL | <400>239 | LTNSELLSLI | <400>240 |
| TNSELLSLIN | <400>241 | NSELLSLIND | <400>242 |
| SELLSLINDM | <400>243 | ELLSLINDMP | <400>244 |
| LLSLINDMPI | <400>245 | LSLINDMPIT | <400>246 |
| SLINDMPITN | <400>247 | LINDMPITND | <400>248 |
| INDMPITNDQ | <400>249 | NDMPITNDQK | <400>250 |
| DMPITNDQKK | <400>251 | MPITNDQKKL | <400>252 |
| PITNDQKKLM | <400>253 | ITNDQKKLMS | <400>254 |
| TNDQKKLMSN | <400>255 | NDQKKLMSNN | <400>256 |
| DQKKLMSNNV | <400>257 | QKKLMSNNVQ | <400>258 |
| KKLMSNNVQI | <400>259 | KLMSNNVQIV | <400>260 |
| LMSNNVQIVR | <400>261 | MSNNVQIVRQ | <400>262 |
| SNNVQIVRQQ | <400>263 | NNVQIVRQQS | <400>264 |
| NVQIVRQQSY | <400>265 | VQIVRQQSYS | <400>266 |
| QIVRQQSYSI | <400>267 | IVRQQSYSIM | <400>268 |
| VRQQSYSIMS | <400>269 | RQQSYSIMSI | <400>270 |
| QQSYSIMSII | <400>271 | QSYSIMSIIK | <400>272 |
| SYSIMSIIKE | <400>273 | YSIMSIIKEE | <400>274 |
| SIMSIIKEEV | <400>275 | IMSIIKEEVL | <400>276 |
| MSIIKEEVLA | <400>277 | SIIKEEVLAY | <400>278 |
| IIKEEVLAYV | <400>279 | IKEEVLAYVV | <400>280 |
| KEEVLAYVVQ | <400>281 | EEVLAYVVQL | <400>282 |
| EVLAYVVQLP | <400>283 | VLAYVVQLPL | <400>284 |
| LAYVVQLPLY | <400>285 | AYVVQLPLYG | <400>286 |
| YVVQLPLYGV | <400>287 | VVQLPLYGVI | <400>288 |
| VQLPLYGVID | <400>289 | QLPLYGVIDT | <400>290 |
| LPLYGVIDTP | <400>291 | PLYGVIDTPC | <400>292 |
| LYGVIDTPCW | <400>293 | YGVIDTPCWK | <400>294 |
| GVIDTPCWKL | <400>295 | VIDTPCWKLH | <400>296 |
| IDTPCWKLHT | <400>297 | DTPCWKLHTS | <400>298 |
| TPCWKLHTSP | <400>299 | PCWKLHTSPL | <400>300 |
| CWKLHTSPLC | <400>301 | WKLHTSPLCT | <400>302 |
| KLHTSPLCTT | <400>303 | LHTSPLCTTN | <400>304 |
| HTSPLCTTNT | <400>305 | TSPLCTTNTK | <400>306 |
| SPLCTTNTKE | <400>307 | PLCTTNTKEG | <400>308 |
| LCTTNTKEGS | <400>309 | CTTNTKEGSN | <400>310 |
| TTNTKEGSNI | <400>311 | TNTKEGSNIC | <400>312 |
| NTKEGSNICL | <400>313 | TKEGSNICLT | <400>314 |
| KEGSNICLTR | <400>315 | EGSNICLTRT | <400>316 |
| GSNICLTRTD | <400>317 | SNICLTRTDR | <400>318 |
| NICLTRTDRG | <400>319 | ICLTRTDRGW | <400>320 |
| CLTRTDRGWY | <400>321 | LTRTDRGWYC | <400>322 |
| TRTDRGWYCD | <400>323 | RTDRGWYCDN | <400>324 |
| TDRGWYCDNA | <400>325 | DRGWYCDNAG | <400>326 |
| RGWYCDNAGS | <400>327 | GWYCDNAGSV | <400>328 |
| WYCDNAGSVS | <400>329 | YCDNAGSVSF | <400>330 |
| CDNAGSVSFF | <400>331 | DNAGSVSFFP | <400>332 |
| NAGSVSFFPQ | <400>333 | AGSVSFFPQA | <400>334 |
| GSVSFFPQAE | <400>335 | SVSFFPQAET | <400>336 |
| VSFFPQAETC | <400>337 | SFFPQAETCK | <400>338 |
| FFPQAETCKV | <400>339 | FPQAETCKVQ | <400>340 |
| PQAETCKVQS | <400>341 | QAETCKVQSN | <400>342 |
| AETCKVQSNR | <400>343 | ETCKVQSNRV | <400>344 |
| TCKVQSNRVF | <400>345 | CKVQSNRVFC | <400>346 |
| KVQSNRVFCD | <400>347 | VQSNRVFCDT | <400>348 |
| QSNRVFCDTM | <400>349 | SNRVFCDTMN | <400>350 |
| NRVFCDTMNS | <400>351 | RVFCDTMNSL | <400>352 |
| VFCDTMNSLT | <400>353 | FCDTMNSLTL | <400>354 |
| CDTMNSLTLP | <400>355 | DTMNSLTLPS | <400>356 |
| TMNSLTLPSE | <400>357 | MNSLTLPSEV | <400>358 |
| NSLTLPSEVN | <400>359 | SLTLPSEVNL | <400>360 |
| LTLPSEVNLC | <400>361 | TLPSEVNLCN | <400>362 |
| LPSEVNLCNV | <400>363 | PSEVNLCNVD | <400>364 |
| SEVNLCNVDI | <400>365 | EVNLCNVDIF | <400>366 |
| VNLCNVDIFN | <400>367 | NLCNVDIFNP | <400>368 |
| LCNVDIFNPK | <400>369 | CNVDIFNPKY | <400>370 |
| NVDIFNPKYD | <400>371 | VDIFNPKYDC | <400>372 |
| DIFNPKYDCK | <400>373 | IFNPKYDCKI | <400>374 |
| FNPKYDCKIM | <400>375 | NPKYDCKIMT | <400>376 |
| PKYDCKIMTS | <400>377 | KYDCKIMTSK | <400>378 |
| YDCKIMTSKT | <400>379 | DCKIMTSKTD | <400>380 |
| CKIMTSKTDV | <400>381 | KIMTSKTDVS | <400>382 |
| IMTSKTDVSS | <400>383 | MTSKTDVSSS | <400>384 |
| TSKTDVSSSV | <400>385 | SKTDVSSSVI | <400>386 |
| KTDVSSSVIT | <400>387 | TDVSSSVITS | <400>388 |
| DVSSSVITSL | <400>389 | VSSSVITSLG | <400>390 |
| SSSVITSLGA | <400>391 | SSVITSLGAI | <400>392 |
| SVITSLGAIV | <400>393 | VITSLGAIVS | <400>394 |
| ITSLGAIVSC | <400>395 | TSLGAIVSCY | <400>396 |
| SLGAIVSCYG | <400>397 | LGAIVSCYGK | <400>398 |
| GAIVSCYGKT | <400>399 | AIVSCYGKTK | <400>400 |
| IVSCYGKTKC | <400>401 | VSCYGKTKCT | <400>402 |
| SCYGKTKCTA | <400>403 | CYGKTKCTAS | <400>404 |
| YGKTKCTASN | <400>405 | GKTKCTASNK | <400>406 |
| KTKCTASNKN | <400>407 | TKCTASNKNR | <400>408 |
| KCTASNKNRG | <400>409 | CTASNKNRGI | <400>410 |
| TASNKNRGII | <400>411 | ASNKNRGIIK | <400>412 |
| SNKNRGIIKT | <400>413 | NKNRGIIKTF | <400>414 |
| KNRGIIKTFS | <400>415 | NRGIIKTFSN | <400>416 |
| RGIIKTFSNG | <400>417 | GIIKTFSNGC | <400>418 |
| IIKTFSNGCD | <400>419 | IKTFSNGCDY | <400>420 |
| KTFSNGCDYV | <400>421 | TFSNGCDYVS | <400>422 |
| FSNGCDYVSN | <400>423 | SNGCDYVSNK | <400>424 |
| NGCDYVSNKG | <400>425 | GCDYVSNKGV | <400>426 |
| CDYVSNKGVD | <400>427 | DYVSNKGVDT | <400>428 |
| YVSNKGVDTV | <400>429 | VSNKGVDTVS | <400>430 |
| SNKGVDTVSV | <400>431 | NKGVDTVSVG | <400>432 |
| KGVDTVSVGN | <400>433 | GVDTVSVGNT | <400>434 |
| VDTVSVGNTL | <400>435 | DTVSVGNTLY | <400>436 |
| TVSVGNTLYY | <400>437 | VSVGNTLYYV | <400>438 |
| SVGNTLYYVN | <400>439 | VGNTLYYVNK | <400>440 |
| GNTLYYVNKQ | <400>441 | NTLYYVNKQE | <400>442 |
| TLYYVNKQEG | <400>443 | LYYVNKQEGK | <400>444 |
| YYVNKQEGKS | <400>445 | YVNKQEGKSL | <400>446 |
| VNKQEGKSLY | <400>447 | NKQEGKSLYV | <400>448 |
| KQEGKSLYVK | <400>449 | QEGKSLYVKG | <400>450 |
| EGKSLYVKGE | <400>451 | GKSLYVKGEP | <400>452 |
| KSLYVKGEPI | <400>453 | SLYVKGEPII | <400>454 |
| LYVKGEPIIN | <400>455 | YVKGEPIINF | <400>456 |
| VKGEPIINFY | <400>457 | KGEPIINFYD | <400>458 |
| GEPIINFYDP | <400>459 | EPIINFYDPL | <400>460 |
| PIINFYDPLV | <400>461 | IINFYDPLVF | <400>462 |
| INFYDPLVFP | <400>463 | NFYDPLVFPS | <400>464 |
| FYDPLVFPSD | <400>465 | YDPLVFPSDE | <400>466 |
| DPLVFPSDEF | <400>467 | PLVFPSDEFD | <400>468 |
| LVFPSDEFDA | <400>469 | VFPSDEFDAS | <400>470 |
| FPSDEFDASI | <400>471 | PSDEFDASIS | <400>472 |
| SDEFDASISQ | <400>473 | DEFDASISQV | <400>474 |
| EFDASISQVN | <400>475 | FDASISQVNE | <400>476 |
| DASISQVNEK | <400>477 | ASISQVNEKI | <400>478 |
| SISQVNEKIN | <400>479 | ISQVNEKINQ | <400>480 |
| SQVNEKINQS | <400>481 | QVNEKINQSL | <400>482 |
| VNEKINQSLA | <400>483 | NEKINQSLAF | <400>484 |
| EKINQSLAFI | <400>485 | KINQSLAFIR | <400>486 |
| INQSLAFIRK | <400>487 | NQSLAFIRKS | <400>488 |
| QSLAFIRKSD | <400>489 | SLAFIRKSDE | <400>490 |
| LAFIRKSDEL | <400>491 | AFIRKSDELL | <400>492 |
| FIRKSDELLH | <400>493 | IRKSDELLHN | <400>494 |
| RKSDELLHNV | <400>495 | KSDELLHNVN | <400>496 |
| SDELLHNVNA | <400>497 | DELLHNVNAG | <400>498 |
| ELLHNVNAGK | <400>499 | LLHNVNAGKS | <400>500 |
| LHNVNAGKST | <400>501 | HNVNAGKSTT | <400>502 |
| NVNAGKSTTN | <400>503 | VNAGKSTTNI | <400>504 |
| NAGKSTTNIM | <400>505 | AGKSTTNIMI | <400>506 |
| GKSTTNIMIT | <400>507 | KSTTNIMITT | <400>508 |
| STTNIMITTI | <400>509 | TTNIMITTII | <400>510 |
| TNIMITTIII | <400>511 | NIMITTIIIV | <400>512 |
| IMITTIIIVI | <400>513 | MITTIIIVII | <400>514 |
| ITTIIIVIIV | <400>515 | TTIIIVIIVI | <400>516 |
| TIIVIIIVIL | <400>517 | IIIVIIVILL | <400>518 |
| IIVIIVILLS | <400>519 | IVIIVILLSL | <400>520 |
| VIIVILLSLI | <400>521 | IIVILLSLIA | <400>522 |
| IVILLSLIAV | <400>523 | VILLSLIAVG | <400>524 |
| ILLSLIAVGL | <400>525 | LLSLIAVGLL | <400>526 |
| LSLIAVGLLL | <400>527 | SLIAVGLLLY | <400>528 |
| LIAVGLLLYC | <400>529 | IAVGLLLYCK | <400>530 |
| AVGLLLYCKA | <400>531 | VGLLLYCKAR | <400>532 |
| GLLLYCKARS | <400>533 | LLLYCKARST | <400>534 |
| LLYCKARSTP | <400>535 | LYCKARSTPV | <400>536 |
| YCKARSTPVT | <400>537 | CKARSTPVTL | <400>538 |
| KARSTPVTLS | <400>539 | ARSTPVTLSK | <400>540 |
| RSTPVTLSKD | <400>541 | STPVTLSKDQ | <400>542 |
| TPVTLSKDQL | <400>543 | PVTLSKDQLS | <400>544 |
| VTLSKDQLSG | <400>545 | TLSKDQLSGI | <400>546 |
| LSKDQLSGIN | <400>547 | SKDQLSGINN | <400>548 |
| KDQLSGINNI | <400>549 | DQLSGINNIA | <400>550 |
| QLSGINNIAF | <400>551 | LSGINNIAFS | <400>552 |
| SGINNIAFSN | <400>553 | | |

43. The agent according to claim 42 wherein said antagonist interacts with a sequence selected from <400>88, <400>89, <400>90, <400>91, <400>92, <400>93 or <400>94.

44. A viral F protein variant comprising a mutation in the intervening peptide sequence wherein said variant exhibits modulated functional activity relative to wild-type F protein or a derivative, homologue, analogue, chemical equivalent or mimetic of said variant.

45. The variant according to claim 44 wherein said variant exhibits down-regulated functional activity relative to wild-type F protein.

46. The variant according to claim 44 or claim 45 wherein said virus is a virus from the family Paramyxoviridae.

47. The variant according to claim 46 wherein said virus is of the sub-family Pneumovirinae.

48. The variant according to claim 47 wherein said virus is respiratory syncytial virus.

49. The variant according to claim 48 wherein said variant comprises a mutation in the cleavage site defined by amino acids RARR (<400>564).

50. The variant according to claim 49 wherein said mutation comprises one or more of the amino acid substitutions selected from the following list:
(i) R106G
(ii) A107Q
(iii) R108G.

51. The variant according to claim 50 wherein said variant comprises the sequence substantially as set forth in <400>565.

52. The variant according to any one of claims 44-48 wherein said variant comprises a multiple amino acid deletion from the intervening peptide sequence.

53. The variant according to claim 52 wherein said amino acid deletion is a partial deletion of the intervening peptide sequence.

54. The variant according to claim 53 wherein said deletion is a deletion of the peptide sequence
RARRELPRFMNYTLNNAKKTNVTLS <400>569

55. The variant according to claim 54 wherein said variant comprises the amino acid sequence substantially as set forth in <400>567.

56. An isolated nucleic acid molecule selected from the list consisting of:
(i) An isolated nucleic acid molecule or derivative or equivalent thereof comprising a nucleotide sequence encoding or complementary to a sequence encoding a viral F protein variant or derivative, homologue, analogue, chemical equivalent or mimetic of said variant, which variant comprises a mutation in the intervening peptide sequence wherein said variant exhibits modulated functional activity relative to wild-type F protein.
(ii) An isolated nucleic acid molecule or derivative or equivalent thereof comprising a nucleotide sequence encoding or complementary to a sequence encoding a viral F protein variant or derivative, homologue, analogue, chemical equivalent or mimetic of said variant, which variant comprises a mutation in the intervening peptide sequence wherein said variant exhibits down-regulated functional activity relative to wild-type F protein.
(iii) An isolated nucleic acid molecule or derivative or equivalent thereof comprising a nucleotide sequence encoding or complementary to a sequence encoding a respiratory syncytial virus F protein or derivative, homologue, analogue, chemical equivalent or mimetic of said variant, which variant comprises a mutation in the cleavage site defined by amino acids RARR wherein said variant exhibits down-regulated functional activity relative to wild-type F protein.
(iv) An isolated nucleic acid molecule or derivative or equivalent thereof comprising a nucleotide sequence encoding or complementary to a sequence encoding a respiratory syncytial virus F protein variant or derivative, homologue, analogue, chemical equivalent or mimetic of said variant, which variant comprises one or more of the amino acid substitutions selected from the following list:
(a) R106G
(b) A107Q
(c) R108G
(v) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises a multiple amino acid deletion from the intervening peptide sequence wherein said variant exhibits down-regulated functional activity relative to wild-type F protein.
(vi) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises a partial deletion of the intervening peptide sequence and more preferably a deletion of the peptide sequence
RARRELPRFMNYTLNNAKKTNVTLS <400>569.
(vii) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises the amino acid sequence substantially as set forth in<400>567.
(viii) An isolated nucleic acid molecule or derivative or analogue thereof comprising a nucleotide sequence encoding or complementary to a sequence encoding a viral F protein variant or derivative, homologue, analogue, chemical equivalent or mimetic of said variant, which variant comprises the amino acid sequence substantially as set forth in<400>565.
(ix) An isolated nucleic acid molecule or derivative or analogue thereof comprising the nucleotide substantially as set forth in<400>568.
(x) An isolated nucleic acid molecule or derivative or analogue thereof comprising the nucleotide substantially as set forth in<400>566.

57. The isolated nucleic acid molecule of claim 56 wherein said virus is a virus from the family Paramyxoviridae.

58. The isolated nucleic acid molecule of claim 57 wherein said virus is of the sub-family Pneumovirinae.

59. The isolated nucleic acid molecule of claim 58 wherein said virus is respiratory syncytial virus.

60. A recombinant viral construct comprising a nucleic acid molecule encoding a viral F protein or derivative thereof, the nucleotide sequence of which nucleic acid molecule comprises codons optimised for expression in a eukaryotic cell, wherein said recombinant viral construct is a effective in inducing, enhancing or otherwise stimulating an immune response to said F protein.

61. A recombinant viral construct comprising a nucleic acid molecule encoding a viral F protein variant or derivative thereof wherein said recombinant viral construct is effective in inducing, enhancing or otherwise stimulating an immune response to said F protein variant.

62. A vaccine comprising a recombinant viral construct which construct comprises a nucleic acid molecule encoding a respiratory syncytial virus F protein or derivative thereof, the nucleic sequence of which nucleic acid molecule is optimised for expression in a eukaryotic cell wherein said recombinant viral construct is effective in inducing, enhancing or otherwise stimulating an immune response to said F protein.

63. A vaccine comprising a recombinant viral construct which construct comprises a nucleic acid molecule encoding a respiratory syncytial virus F protein variant or derivative thereof, wherein said recombinant viral construct is effective in inducing, enhancing or otherwise stimulating an immune response to said F protein variant.

64. A vaccine according to claim 62 or claim 63 wherein said nucleotide sequence is defined in one of <400>5, <400>6, <400>566 or <400>568.

65. Use of the agent according to any one of claims 41-43 or identified in accordance with the method of any one of claims 34-40 to modulate F protein functional activity.

66. Use of the agent according to any one of claims 41-43 or identified in accordance with the method of any one of claims 34-40 in the therapeutic and/or prophylactic treatment of conditions characterised by infection with a negative sense single stranded RNA virus.

67. A method of modulating at least one functional activity associated with a viral F protein in a subject, said method comprising introducing into said subject an effective amount of a F protein modulatory agent according to any one of claims 41-43 or identified in accordance with the method of any one of claims 34-40 for a time and under conditions sufficient for said agent to interact with said F protein.

68. The method according to claim 68 wherein said functional activity is F protein mediated host cell virion fusion and/or virion budding and said modulating is down-regulation.

69. A method of modulating at least one functional activity associated with a viral F protein, said method comprising contacting said viral F protein with an effective amount of a F protein modulatory agent according to any one of claims 41-43 or identified in accordance with the method of any one of claims 34-40 for a time and under conditions sufficient for said agent to interact with said F protein.

70. A method for the treatment and/or prophylaxis of a condition characterised by infection with a negative sense single stranded RNA virus in a subject, said method comprising administering to said subject an effective amount of an agent according to any one of claims 41-43 or identified in accordance with the method of any one of claims 34-40 which agent is capable of down-regulating at least one functional activity of the F protein expressed by said virus, for a time and under conditions sufficient for said agent to interact with said F protein.

71. A method for the treatment and/or prophylaxis of a condition characterised by infection with a negative sense single stranded virus in a subject, said method comprising administering to said subject an effective amount of a composition comprising an F protein or derivative thereof, F protein variant or derivative thereof and/or a nucleic acid molecule encoding said F protein or F protein variant or a derivative, homologue, analogue, chemical equivalent or mimetic of said protein or nucleic acid molecule for a time and under conditions sufficient for said composition to down regulate said viral F protein functional activity.

72. The method according to claim 71 wherein said subject is a mammal.

73. The method according to claim 72 wherein said mammal is a human.

74. Use of an agent capable of modulating at least one functional activity of a viral F protein which agent according to any one of claims 41-43 or identified in accordance with the method of any one of claims 3440 in the manufacture of a medicament for the treatment and/or prophylaxis of a condition characterised by infection with negative sense single stranded RNA virus.

75. Use of a composition comprising an F protein or derivative thereof, F protein variant or derivative thereof, nucleic acid molecule encoding said F protein or F protein variant according to any one of claims 41-43 or identified in accordance with the method of any one of claims 34-40 or a derivative, homologue, analogue, chemical equivalent or mimetic of said protein or nucleic acid molecule, in the manufacture of a medicament for the treatment and/or prophylaxis of a condition characterised by infection with a negative sense single stranded RNA virus.

76. Use of an agent, which agent, according to any one of claims 41-43 or identified in accordance with the method of any one of claims 34-40 in the manufacture of a medicament for the modulation of at least one viral F protein associated functional activity.

77. Agents for use in modulating the functional activity of a viral F protein wherein said agent is identified in accordance with the method of any one of claims 34-40.

78. Agents for use in the treatment and/or prophylaxis of a condition characterised by infection with a negative sense single stranded RNA virus wherein said agent is identified in accordance with the methods of any one of claims 34-40.

79. A composition comprising an F protein or derivative thereof, F protein variant or derivative thereof, a nucleic acid molecule encoding said F protein or F protein variant or a derivative, homologue, analogue, chemical equivalent or mimetic of said protein or nucleic acid molecule for use in the treatment and/or prophylaxis of a condition characterised by infection with a negative sense single stranded RNA virus.
